(19) United States
(12) Patent Application Publication
(10) Pub. No.: US 2006/0084592 A1
(43) Pub. Date: Apr. 20, 2006

(54) Peptide boronic acid inhibitors

(75) Inventor: Oliver Vimpany Arnold Boucher, London (GB)

Correspondence Address: KLARQUIST SPARKMAN, LLP, 121 SW SALMON STREET, SUITE 1600, PORTLAND, OR, 97204, US

(73) Assignee: Trigen Limited

(21) Appl. No.: 11/077,620

(22) Filed: Mar. 9, 2005

Related U.S. Application Data
(63) Continuation-in-part of application No. 10/659,179, filed on Sep. 9, 2003.

(30) Foreign Application Priority Data
Sep. 9, 2002 (GB) GB0220764.5
Sep. 9, 2002 (GB) GB0220822.1
Apr. 4, 2003 (GB) GB0307817.7
May 16, 2003 (GB) GB0311237.2
Jul. 4, 2003 (GB) GB0315691.6

(51) Int. Cl. A61K 38/10; A61K 38/08; C07K 7/08; C07K 7/06

(52) U.S. Cl. 514/2; 530/300

(57) ABSTRACT

A pharmaceutically acceptable base addition salt of an organoboronic acid of formula (XXX): 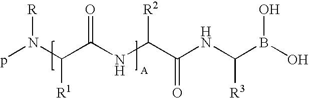 wherein: P is hydrogen or an amino-group protecting moiety; R is hydrogen or alkyl; A is 0, 1 or 2;
R¹, R² and R³ are independently hydrogen, alkyl, cycloalkyl, aryl or —CH₂—R⁵;
R⁵, in each instance, is one of aryl, aralkyl, alkaryl, cycloalkyl, heterocyclyl, heteroaryl, or —W—R⁶, where W is a chalcogen and R⁶ is alkyl; and
where the ring portion of any of said aryl, aralkyl, alkaryl, cycloalkyl, heterocyclyl, or heteroaryl in R¹, R², R³ or R⁵ can be optionally substituted.

This application is a continuation-in-part of U.S. application Ser. No. 10/659,179, filed Sep. 9, 2003, which is herein incorporated by reference, which claims the benefit of U.K. Application No. GB 0220764.5, filed Sep. 9, 2002, U.K. Application No. GB 0220822.1, filed Sep. 9, 2002, U.K. Application No. GB 0307817.7, filed Apr. 4, 2003, U.K. Application No. GB 0311237.2, filed May 16, 2003, and U.K. Application No. GB 0315691.6, filed Jul. 4, 2003, all of which are herein incorporated by reference.

U.S. Publication No. US-2004-0138175-A1 and U.S. Publication No. US-2004-0147453-A1 are herein incorporated by reference.

### BACKGROUND

The present disclosure relates to boronic acids, particularly peptide boronic acids. It relates also to pharmaceutically useful products obtainable from organoboronic acids. The disclosure also relates to the use of members of the aforesaid class of products, to their formulation, their preparation, their synthetic intermediates and to other subject matter.

Boronic Acid Compounds

It has been known for some years that boronic acid compounds and their derivatives, e.g. esters, have biological activities, notably as inhibitors or substrates of proteases. For example, Koehler et al. Biochemistry 10:2477, 1971 report that 2-phenylethane boronic acid inhibits the serine protease chymotrypsin at millimolar levels. The inhibition of chymotrypsin and subtilisin by arylboronic acids (phenylboronic acid, m-nitro-phenylboronic acid, m-aminophenylboronic acid, m-bromophenylboronic acid) is reported by Phillip et al, Proc. Nat Acad. Sci. USA 68:478-480, 1971. A study of the inhibition of subtilisin Carlsberg by a variety of boronic acids, especially phenyl boronic acids substituted by Cl, Br, CH₃, H₂N, MeO and others, is described by Seufer-Wasserthal et al, Biorg. Med. Chem. 2(1):35-48, 1994.

In describing inhibitors or substrates of proteases, P1, P2, P3, etc. designate substrate or inhibitor residues which are amino-terminal to the scissile peptide bond, and S1, S2, S3, etc., designate the corresponding subsites of the cognate protease in accordance with: Schechter, I. and Berger, A. On the Size of the Active Site in Proteases, Biochem. Biophys. Res. Comm., 27:157-162, 1967. In thrombin, the S1 binding site or “specificity pocket” is a well defined slit in the enzyme, whilst the S2 and S3 binding subsites (also respectively called the proximal and distal hydrophobic pockets) are hydrophobic and interact strongly with, respectively, Pro and (R)-Phe, amongst others.

Pharmaceutical research into serine protease inhibitors has moved from the simple arylboronic acids to boropeptides, i.e. peptides containing a boronic acid analogue of an α-amino carboxylic acid. The boronic acid may be derivatised, often to form an ester. Shenvi (EP-A-145441 and U.S. Pat. No. 4,499,082) disclosed that peptides containing an α-aminoboronic acid with a neutral side chain were effective inhibitors of elastase and has been followed by numerous patent publications relating to boropeptide inhibitors of serine proteases. Specific, tight binding boronic acid inhibitors have been reported for elastase (Kᵢ, 0.25 nM), chymotrypsin (Kᵢ, 0.25 nM), cathepsin G (Kᵢ, 21 nM), α-lytic protease (Kᵢ, 0.25 nM), dipeptidyl aminopeptidase type IV (Kᵢ, 16 pM) and more recently thrombin (Ac-D-Phe-Pro-boroArg-OH (DuP 714 initial Kᵢ 1.2 nM).

Claeson et al (U.S. Pat. No. 5,574,014 and others) and Kakkar et al (WO 92/07869 and family members including U.S. Pat. No. 5,648,338) disclose thrombin inhibitors having a neutral C-terminal side chain, for example an alkyl or alkoxyalkyl side chain.

Modifications of the compounds described by Kakkar et al are included in WO 96/25427, directed to peptidyl serine protease inhibitors in which the P2-P1 natural peptide linkage is replaced by another linkage. As examples of non-natural peptide linkages may be mentioned —CO₂—, —CH₂O—, —NHCO—, —CHYCH₂—, —CH═CH—, —CO(CH₂)ₚCO— where p is 1, 2 or 3, —COCHY—, —CO₂—CH₂NH—, —CHY—NX—, —N(X)CH₂—N(X)CO—, —CH═C(CN)CO—, —CH(OH)—NH—, —CH(CN)—N H—, —CH(OH)—CH₂— or —NH—CHOH—, where X is H or an amino protecting group and Y is H or halogen, especially F. Particular non-natural peptide linkages are —CO₂— or —CH₂O—.

Metternich (EP 471651 and U.S. Pat. No. 5,288,707, the latter being assigned to Trigen Limited) discloses variants of Phe-Pro-BoroArg boropeptides in which the P3 Phe is replaced by an unnatural hydrophobic amino acid such as trimethylsilylalanine, p-tert.butyl-diphenyl-silyloxymethyl-phenylalanine or p-hydroxymethylphenylalanine and the P1 side chain may be neutral (alkoxyalkyl, alkylthioalkyl or trimethylsilylalkyl).

The replacement of the P2 Pro residue of borotripeptide thrombin inhibitors by an N-substituted glycine is described in Fevig I M et al Bioorg. Med. Chem. 8: 301-306 and Rupin A et al

Thromb. Haemost. 78(4):1221-1227, 1997. See also U.S. Pat. No. 5,585,360 (de Nanteuil et al). Amparo (WO 96/20698 and family members including U.S. Pat. No. 5,698,538) discloses peptidomimetics of the structure Aryl-linker-Boro(Aa), where Boro(Aa) may be an aminoboronate residue with a non-basic side chain, for example BoroMpg. The linker is of the formula

(CH₂)ₘCONR— (where m is 0 to 8 and R is H or certain organic groups) or analogues thereof in which the peptide linkage —CONR— is replaced by —CSNR—, —SO₂NR—, —CO₂—, —C(S)O— or —SO₂O—. Aryl is phenyl, naphthyl or biphenyl substituted by one, two or three moieties selected from a specified group. Most typically these compounds are of the structure Aryl-(CH₂)ₙ—CONH—CHR²—BY¹Y², where R² is for example a neutral side chain as described above and n is 0 or 1.

Non-peptide boronates have been proposed as inhibitors of proteolytic enzymes in detergent compositions. WO 92/19707 and WO 95/12655 report that arylboronates can be used as inhibitors of proteolytic enzymes in detergent compositions. WO 92/19707 discloses compounds substituted meta to the boronate group by a hydrogen bonding group, especially acetamido (—NHCOCH₃), sufonamido (—NHSO₂CH₃) and alkylamino. WO 95/12655 teaches that ortho-substituted compounds are superior.

Boronate enzyme inhibitors have wide application, from detergents to bacterial sporulation inhibitors to pharmaceuticals. In the pharmaceutical field, there is patent literature describing boronate inhibitors of serine proteases, for example thrombin, factor Xa, kallikrein, elastase, plasmin as well as other serine proteases like prolyl endopeptidase and Ig AI Protease. Thrombin is the last protease in the coagulation pathway and acts to hydrolyse four small peptides form each molecule of fibrinogen, thus deprotecting its polymerisation sites. Once formed, the linear fibrin polymers may be cross-linked by factor XIIIa, which is itself activated by thrombin. In addition, thrombin is a potent activator of platelets, upon which it acts at specific receptors. Thrombin also potentiates its own production by the activation of factors V and VIII.

Other aminoboronate or peptidoboronate inhibitors or substrates of serine proteases are described in:
U.S. Pat. No. 4,935,493
EP 341661
WO 94/25049
WO 95/09859
WO 96/12499
WO 96/20689
Lee S-L et al, Biochemistry 36:13180-13186, 1997
Dominguez C et al, Bioorg. Med. Chem. Lett. 7:79-84, 1997
EP 471651
WO 94/20526
WO 95/20603
WO97/05161
U.S. Pat. No. 4,450,105
U.S. Pat. No. 5,106,948
U.S. Pat. No. 5,169,841.

Peptide boronic acid inhibitors of hepatic C virus protease are described in WO 01/02424.

Matteson D S Chem. Rev. 89: 1535-1551, 1989 reviews the use of α-halo boronic esters as intermediates for the synthesis of inter alia amino boronic acids and their derivatives. Matteson describes the use of pinacol boronic esters in non-chiral synthesis and the use of pinanediol boronic esters for chiral control, including in the synthesis of amino and amido boronate esters.

Contreras et al J. Organomet Chem. 246: 213-217, 1983 describe how intramolecular N→B coordination was demonstrated by spectroscopic studies on cyclic boronic esters prepared by reacting Me₂CHCMe₂-BH₂ with diethanolamines.

Boronic acid and ester compounds have displayed promise as inhibitors of the proteasome, a multicatalytic protease responsible for the majority of intracellular protein turnover. Ciechanover, Cell, 79:13-21, 1994, teaches that the proteasome is the proteolytic component of the ubiquitin-proteasome pathway, in which proteins are targeted for degradation by conjugation to multiple molecules of ubiquitin. Ciechanover also teaches that the ubiquitin-proteasome pathway plays a key role in a variety of important physiological processes.

Adams et al, U.S. Pat. No. 5,780,454 (1998), U.S. Pat. No. 6,066,730 (2000), U.S. Pat. No. 6,083,903 (2000) and equivalent WO 96/13266, and U.S. Pat. No. 6,297,217 (2001) describe peptide boronic ester and acid compounds useful as proteasome inhibitors. These documents also describe the use of boronic ester and acid compounds to reduce the rate of muscle protein degradation, to reduce the activity of NF-κB in a cell, to reduce the rate of degradation of p53 protein in a cell, to inhibit cyclin degradation in a cell, to inhibit the growth of a cancer cell, to inhibit antigen presentation in a cell, to inhibit NF-κB dependent cell adhesion, and to inhibit HIV replication. Brand et al, WO 98/35691, teaches that proteasome inhibitors, including boronic acid compounds, are useful for treating infarcts such as occur during stroke or myocardial infarction. Elliott et al, WO 99/15183, teaches that proteasome inhibitors are useful for treating inflammatory and autoimmune diseases.

Unfortunately, organoboronic acids can be relatively difficult to obtain in analytically pure form. Thus, alkylboronic acids and their boroxines are often air-sensitive. Korcek et al, J. Chem. Soc. Perkin Trans. 2:242, 1972, teaches that butylboronic acid is readily oxidized by air to generate 1-butanol and boric acid.

It is known that derivatisation of boronic acids as cyclic esters provides oxidation resistance. For example, Martichonok V et al J. Am. Chem. Soc. 118: 950-958, 1996 state that diethanolamine derivatisation provides protection against possible boronic acid oxidation. U.S. Pat. No. 5,681,978 (Matteson D S et al) teaches that 1,2-diols and 1,3 diols, for example pinacol, form stable cyclic boronic esters that are not easily oxidised.

Wu et al, J. Pharm. Sci., 89:758-765, 2000, discuss the stability of the compound N-(2-pyrazine) carbonyl-phenylalanine-leucine boronic acid (bortezomib), an anti-cancer agent. It is described how “during an effort to formulate [bortezomib] for parenteral administration, the compound showed erratic stability behaviour”. The degradation pathways were investigated and it was concluded that the degradation was oxidative, the initial oxidation being attributed to peroxides or molecular oxygen and its radicals.

WO 02/059131 discloses boronic acid products which are described as stable. In particular, these products are certain boropeptides and/or boropeptidomimetics in which the boronic acid group has been derivatised with a sugar. The disclosed sugar derivatives, which have hydrophobic amino acid side chains, are of the formula 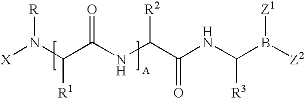
wherein:
X is hydrogen or an amino-group protecting moiety;
R is hydrogen or alkyl;
A is 0, 1 or 2;
R¹, R² and R³ are independently hydrogen, alkyl, cycloalkyl, aryl or —CH₂—R⁵;
R⁵, in each instance, is one of aryl, aralkyl, alkaryl, cycloalkyl, heterocyclyl, heteroaryl, or —W—R⁶, where W is a chalcogen and R⁶ is alkyl;
where the ring portion of any of said aryl, aralkyl, alkaryl, cycloalkyl, heterocyclyl, or heteroaryl in R¹, R², R³ or R⁵ can be optionally substituted; and
Z¹ and Z² together form a moiety derived from a sugar, wherein the atom attached to boron in each case is an oxygen atom.

Some of the disclosed compounds are sugar derivatives of bortezomib (see above).

Many drugs comprise an active moiety which is a carboxylic acid. There are a number of differences between carboxylic acids and boronic acids, whose effects on drug delivery, stability and transport (amongst others) have not been investigated. One feature of trivalent boron compounds is that the boron atom is sp² hybridised, which leaves an empty 2p_{z} orbital on the boron atom. A molecule of the type BX₃ can therefore act as an electron-pair acceptor, or Lewis acid. It can use the empty 2p_{z} orbital to pick up a pair of nonbonding electrons from a Lewis base to form a covalent bond. BF₃ therefore reacts with Lewis bases such as NH₃ to form acid-base complexes in which all of the atoms have a filled shell of valence electrons.

Boric acid, accordingly, can act as a Lewis acid, accepting OH⁻:
B(OH)₃+H₂→B(OH)₄⁻+H⁺

Further, boronic acids of the type RB(OH)₂ are dibasic and have two pKa's. Another point of distinction about boron compounds is the unusually short length of bonds to boron, for which three factors may be responsible:

1. Formation of pn-pn bonds;

2. Ionic-covalent resonance;

3. Reduced repulsions between non-bonding electrons.

The presumed equilibria of boronic and carboxylic acids in aqueous KOH are shown below (excluding formation of RBO₂²): 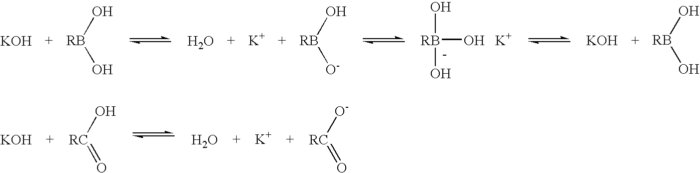
Aminoboronate Synthesis

It is known in the prior art to synthesise TRI 50 c esters via the following process:

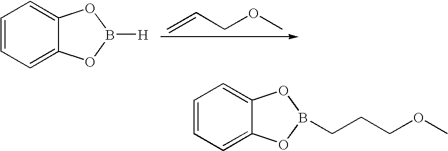

The product of the above step is then converted by known methods to, for example, TRI 50b. See for example Deadman I et al, J. Medicinal Chemistry 1995, 38, 1511-1522.

Thrombosis

Hemostasis is the normal physiological condition of blood in which its components exist in dynamic equilibrium. When the equilibrium is disturbed, for instance following injury to a blood vessel, certain biochemical pathways are triggered leading, in this example, to arrest of bleeding via clot formation (coagulation). Coagulation is a dynamic and complex process in which proteolytic enzymes such as thrombin play a key role. Blood coagulation may occur through either of two cascades of zymogen activations, the extrinsic and intrinsic pathways of the coagulation cascade. The last protease in each pathway is thrombin which catalyses the polymerization of fibrinogen monomers to fibrin polymer. In addition, thrombin is a potent activator of platelets, upon which it acts at specific receptors. Thrombin activation of platelets leads to aggregation of the cells and secretion of additional factors that further accelerate the creation of a hemostatic plug. Thrombin also potentiates its own production by the activation of factors V and VIII (see Hemker and Beguin in: Jolles, et. al., “Biology and Pathology of Platelet Vessel Wall Interactions,” pp. 219-26 (1986), Crawford and Scrutton in: Bloom and Thomas, “Haemostasis and Thrombosis,” pp. 47-77, (1987), Bevers, et. al., Eur. J. Biochem. 122:429-36, 1982, Mann, Trends Biochem. Sci 12:229-33, 1987).

Proteases are enzymes which cleave proteins at specific peptide bonds. Cuypers et al., J. Biol. Chem. 257:7086, 1982, and the references cited therein, classify proteases on a mechanistic basis into five classes: serine, cysteinyl or thiol, acid or aspartyl, threonine and metalloproteases. Members of each class catalyse the hydrolysis of peptide bonds by a similar mechanism, have similar active site amino acid residues and are susceptible to class-specific inhibitors. For example, all serine proteases that have been characterised have an active site serine residue.

The coagulation proteases thrombin, factor Xa, factor VIIa, and factor IXa are serine proteases having trypsin-like specificity for the cleavage of sequence-specific Arg-Xxx peptide bonds. As with other serine proteases, the cleavage event begins with an attack of the active site serine on the scissile bond of the substrate, resulting in the formation of a tetrahedral intermediate. This is followed by collapse of the tetrahedral intermediate to form an acyl enzyme and release of the amino terminus of the cleaved sequence. Hydrolysis of the acyl enzyme then releases the carboxy terminus.

The management of thrombosis commonly involves the use of antiplatelet drugs (inhibitors of platelet aggregation) to control future thrombogenesis and thrombolytic agents to lyse the newly formed clot, either or both such agents being used in conjunction or combination with anticoagulants. Anticoagulants are used also preventatively (prophylactically) in the treatment of patients thought susceptible to thrombosis.

Currently, two of the most effective classes of drugs in clinical use as anticoagulants are the heparins and the vitamin K antagonists. The heparins are ill-defined mixtures of sulfated polysaccharides that bind to, and thus potentiate, the action of antithrombin III. Antithrombin III is a naturally occurring inhibitor of the activated clotting factors IXa, Xa, XIa, thrombin and probably XIIa (see Jaques, Pharmacol. Rev. 31:99-166, 1980). The vitamin K antagonists, of which warfarin is the most well-known example, act indirectly by inhibiting the post-ribosomal carboxylations of the vitamin K dependent coagulation factors II, VII, IX and X (see Hirsch, Semin. Thromb. Hemostasis 12:1-11, 1986). While effective therapies for the treatment of thrombosis, heparins and vitamin K antagonists have the unfortunate side effects of bleeding, heparin-induced thrombocytopenia (in the case of heparin) and marked interpatient variability, resulting in a small and unpredictable therapeutic safety margin.

The use of direct acting inhibitors of thrombin and other serine protease enzymes of the coagulation system is expected to alleviate these problems. To that end, a wide variety of serine protease inhibitors have been tested, including boropeptides, i.e. peptides containing a boronic acid analogue of an α-amino acid. Whilst direct acting boronic acid thrombin inhibitors have been discussed earlier in this specification, they are further described in the following section.

Neutral P1 Residue Boropeptide Thrombin Inhibitors

Claeson et al (U.S. Pat. No. 5,574,014 and others) and Kakkar et al (WO 92/07869 and family members including U.S. Pat. No. 5,648,338) disclose lipophilic thrombin inhibitors having a neutral (uncharged) C-terminal (P1) side chain, for example an alkoxyalkyl side chain.

The Claeson et al and Kakkar et al patent families disclose boronate esters containing the amino acid sequence D-Phe-Pro-BoroMpg [(R)-Phe-Pro-BoroMpg], which are highly specific inhibitors of thrombin. Of these compounds may be mentioned in particular Cbz-(R)-Phe-Pro-BoroMpg-OPinacol (also known as TRI 50b).

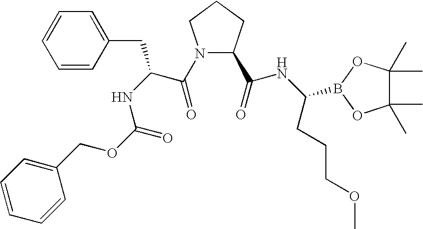

(R,S,R)-TRI 50b Cbz-(R)-Phe-(S)-Pro-R)boroMpg-Pinacol

The corresponding free boronic acid is known as TRI 50c. For further information relating to TRI 50b and related compounds, the reader is referred to the following documents:
Elgendy S et al., in The Design of Synthetic Inhibitors of Thrombin, Claeson G et al Eds, Advances in Experimental Medicine, 340:173-178, 1993.
Claeson G et al, Biochem J. 290:309-312, 1993
Tapparelli C et al, J Biol Chem, 268:4734-4741, 1993
Claeson G, in The Design of Synthetic Inhibitors of Thrombin, Claeson G et al Eds, Advances in Experimental Medicine, 340:83-91, 1993
Phillip et al, in The Design of Synthetic Inhibitors of Thrombin, Claeson G et al Eds, Advances in Experimental Medicine, 340:67-77, 1993
Tapparelli C et al, Trends Pharmacol. Sci 14:366-376, 1993
Claeson G, Blood Coagulation and Fibrinolysis 5:411-436, 1994
Elgendy et al, Tetrahedron 50:3803-3812, 1994
Deadman J et al, J. Enzyme Inhibition 9:29-41, 1995
Deadman J et al, J. Medicinal Chemistry 38:1511-1522, 1995.

TRI 50b is considered to be a prodrug for TRI 50c, which is the active principal in vivo. The tripeptide sequence of TRI 50c has three chiral centres. The Phe residue is considered to be of (R)-configuration and the Pro residue of natural (S)-configuration, at least in compounds with commercially useful inhibitor activity; the Mpg residue is believed to be of (R)-configuration in isomers with commercially useful inhibitor activity. Thus, the active, or most active, TRI 50c stereoisomer is considered to be of R,S,R configuration and may be represented as:

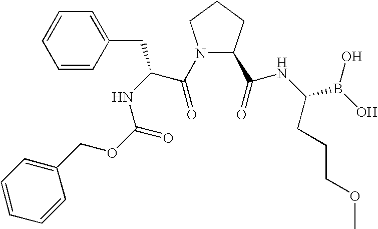

(R,S,R)-TRI 50c Cbz-(R)-Phe-(S)-Pro-(R)-Mpg-B(OH)₂

Guide to the Specification

This specification, as described in more detail below, concerns in particular various subject matters relating to novel compounds and compositions. For convenience, the term “Novel Products” is sometimes (but not always) used in the description to refer to products comprising these novel compounds and compositions; for example the term is used in headings.

The subject matters of the disclosure include synthetic methods devised in an earlier part of the research and development programme concerning the Novel Products, which methods generated one or more impurities and were otherwise not usable as such on an industrial scale. The term “Synthetic Methods I” is sometimes (but not always) used in the description to refer to such earlier methods; for example the term is used in headings. The subject matters relating to the Novel Products also include various aspects of subsequently devised synthetic techniques for making the novel compounds (or intermediates therefor) and relatively high purity products obtainable using these techniques; the term “Synthetic Methods II” is sometimes (but not always) used in the description to refer to such subsequent methods; for example the term is used in headings. At least in certain aspects, Synthetic Methods II represent a sub-set of Synthetic Methods I. The specific products of Synthetic Methods II are for convenience sometimes referred to as “High Purity Products”. The High Purity Products are a sub-set of the Novel Products.

The phrases Novel Products, Synthetic Methods I, Synthetic Methods II and High Purity Products are used solely for convenience and are not to be understood as limiting the scope of the invention, which includes the entire subject matter of the disclosure, including all materials, species, processes and uses thereof.

### BRIEF SUMMARY OF THE DISCLOSURE

1. Novel Products

It has been discovered that TRI 50b tends to hydrolyse. Thus in acid conditions, for example of an HPLC assay, TRI 50b is converted to the acid form with a short half life, which implies potential hydrolysis in parenteral preparations containing water into species, comprising the free acid and its corresponding boronate anions in equilibrium therewith, taught in the literature to be unstable to degradation via de-boronation (carbon-boron bond cleavage), by an oxidative pathway (see e.g. Wu et al, discussed above).

The instability of TRI 50b to hydrolysis also presents potential disadvantages in preparation of the compound and its formulation, as well as in the storage of pharmaceutical formulations containing it.

TRI 50c suffers further from instability, in that there is a problematic tendency for the boropeptide moiety itself to degrade via de-boronation (carbon-boron bond cleavage), such deboronation being taught by the literature to be oxidative (e.g. Wu et al, discussed above). The level of degradation can be remarkably high.

The properties discussed above of TRI 50b and TRI 50c will not be restricted to such compounds but will be shared by other boropeptide esters and acids, even if the properties of such other boropeptides differ quantitatively. For example commercial bortezomib (mannitol ester of N-(2-pyrazine) carbonyl-phenylalanine-leucine boronic acid, sold under the registered trade mark VELCADE) is required to be stored at controlled room temperature in its original package to protect from light and, when reconstituted must not be stored for more than 8 hours when exposed to normal indoor lighting (see Velcade US Package Insert dated May 13, 2003).

The present disclosure is predicated on a novel and non-obvious alternative form of boronic acid medicaments to sugar esters.

It is contemplated that embodiments of the disclosure provide stabilised forms of boronic acid drug, and that embodiments provide a solution to the problem of boronate diol ester and especially TRI50b instability which also provides the corresponding boronic acid with resistance to deboronation.

Embodiments of the present disclosure are predicated on, amongst other things, the finding that certain organoboronic acid products are indicated to be of enhanced stability.

The benefits of the present disclosure include a solution to the problem of boronate diol ester and especially TRI 50b instability, that is to say the presently disclosed products provide inter alia pharmacologically active compounds which are more stable than TRI 50b and other comparable esters in the sense of stability to hydrolysis. The disclosure further includes a solution to the problem of organoboronic acid instability, that is to say the presently disclosed products provide inter alia pharmacologically active compounds which are more stable to deboronation than TRI 50c. The stability provided within the framework of the disclosure is not absolute but is improved relative to the comparator compounds. The benefits offered by the disclosure further include the provision of products which have an unexpected usefulness in parenteral formulations.

There is disclosed an amino boronic acid derivative which avoids the disadvantages of pinacol esters. The disclosure further includes a peptide boronic acid derivative which is indicated to be of enhanced stability. In particular, the disclosure includes amongst other subject matter boronic acid derivatives which are of relative stability to hydrolysis and deboronation and are useful in parenteral formulations for inhibiting thrombin.

The disclosure concerns a pharmaceutically acceptable base addition salt of organoboronic acid drugs, and more specifically hydrophobic boropeptides (e.g. di- or tri-peptides), for example thrombin inhibitors and proteasome inhibitors having a non-basic P1 group. As a class, such salts are not only contrary to the direction of the prior art but additionally have an improved level of stability which cannot be explained or predicted on the basis of known chemistry.

An aspect of the invention relates to base addition salts of boronic acid drugs, in particular those having of the formula (XXX): 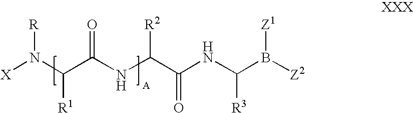
wherein:
X is hydrogen or an amino-group protecting moiety;
R is hydrogen or alkyl;
A is 0, 1 or 2;
R¹, R² and R³ are independently hydrogen, alkyl, cycloalkyl, aryl or —CH₂—R⁵;
R⁵, in each instance, is one of aryl, aralkyl, alkaryl, cycloalkyl, heterocyclyl, heteroaryl, or —W—R⁶, where W is a chalcogen and R⁶ is alkyl;
where the ring portion of any of said aryl, aralkyl, alkaryl, cycloalkyl, heterocyclyl, or heteroaryl in R¹, R², R³ or R⁵ can be optionally substituted; and
Z¹ and Z² together form a moiety derived from a sugar, wherein the atom attached to boron in each case is an oxygen atom.

A particular example of compounds falling within this class is bortezomib (Velcade®), i.e. N-(2-pyrazine) carbonyl-phenylalanine-leucine boronic acid.

The compound of formula (XXX) are non-obvious in their own right. Additionally, the disclosure includes a method of stabilising an organoboronic acid of formula (X)X), comprising providing it in the form of a pharmaceutically acceptable base addition salt thereof

Also included is a method of formulating an organoboronic acid drug of formula (XXX) to increase the stability of the drug species, comprising formulating the acid in the form of a pharmaceutically acceptable base addition salt thereof, the salt being an acid salt.

The disclosure further relates to base addition salts of boronic acids which have a neutral aminoboronic acid residue capable of binding to the thrombin S1 subsite linked through a peptide linkage to a hydrophobic moiety capable of binding to the thrombin S2 and S3 subsites. In a first embodiment, there is disclosed a parenteral pharmaceutical formulation that includes a pharmaceutically acceptable base addition salt of a boronic acid of, for example, formula (I): 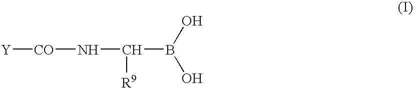
wherein
Y comprises a hydrophobic moiety which, together with the aminoboronic acid residue —NHCH(R⁹)—B(OH)₂, has affinity for the substrate binding site of thrombin; and

R⁹ is a straight chain alkyl group interrupted by one or more ether linkages (e.g. 1 or 2) and in which the total number of oxygen and carbon atoms is 3, 4, 5 or 6 (e.g. 5) or R⁹ is —(CH₂)ₘ—W where m is 2, 3, 4 or 5 (e.g. 4) and W is —OH or halogen (F, Cl, Br or I). R⁹ is an alkoxyalkyl group in one subset of compounds, e.g. alkoxyalkyl containing 4 carbon atoms.

Disclosed as certain examples are base addition salts of hydrophobic boronic acid inhibitors of thrombin. Such inhibitors may contain hydrophobic amino acids, and this class of amino acids includes those whose side chain is hydrocarbyl, hydrocarbyl containing an in-chain oxygen and/or linked to the remainder of the molecule by an in-chain oxygen or heteroaryl, or any of the aforesaid groups when substituted by hydroxy, halogen or trifluoromethyl. Representative hydrophobic side chains include alkyl, alkoxyalkyl, either of the aforesaid when substituted by at least one aryl or heteroaryl, aryl, heteroaryl, aryl substituted by at least one alkyl and heteroaryl substituted by at least one alkyl. Proline and other imino acids which are ring-substituted by nothing or by one of the moieties listed in the previous sentence are also hydrophobic.

Some hydrophobic side chains contain from 1 to 20 carbon atoms, e.g. non-cyclic moieties having 1, 2, 3 or 4 carbon atoms. Side chains comprising a cyclic group typically but not necessarily contain from 5 to 13 ring members and in many cases are phenyl or alkyl substituted by one or two phenyls.

Included are inhibitors which contain hydrophobic non-peptide moieties, which are typically based on moieties which may form a side chain of a hydrophobic amino acid, as described above.

Hydrophobic compounds may contain, for example, one amino group and/or one acid group (e.g. —COOH, —B(OH)₂). Generally, they do not contain multiple polar groups of any one type.

The disclosure comprises base addition salts of hydrophobic boronic acid inhibitors of thrombin, and therefore includes such salts of peptide boronic acids which have a partition coefficient between 1-n-octanol and water expressed as log P of greater than 1.0 at physiological pH and 25° C. Some peptide boronic acids useful in the invention have a partition coefficient of at least 1.5. A class of hydrophobic peptide boronic acids useful in the invention has a partition coefficient of no more than 5.

Some sub-classes of hydrophobic organoboronic acids are those described by Formulae (I) and (III) below, under the heading “Detailed Description of Several Examples”.

Also disclosed as another embodiment is a pharmaceutically acceptable base addition salt of a peptide boronic acid of formula (II): 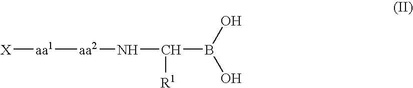
where:

X is a moiety bonded to the N-terminal amino group and may be H to form NH₂. The identity of X is not critical but may be a particular X moiety described above. In one example there may be mentioned benzyloxycarbonyl.

aa¹ is an amino acid having a hydrocarbyl side chain containing no more than 20 carbon atoms (e.g. up to 15 and optionally up to 13 C atoms) and comprising at least one cyclic group having up to 13 carbon atoms. In certain examples, the cyclic group(s) of aa¹ have/has 5 or 6 ring members. For instance, the cyclic group(s) of aa¹ may be aryl groups, particularly phenyl. Typically, there are one or two cyclic groups in the aa¹ side chain. Certain side chains comprise, or consist of, methyl substituted by one or two 5- or 6-membered rings.

More particularly, aa¹ is Phe, Dpa or a wholly or partially hydrogenated analogue thereof. The wholly hydrogenated analogues are Cha and Dcha.

aa² is an imino acid having from 4 to 6 ring members. Alternatively, aa² is Gly N-substituted by a C₃-C₁₃ hydrocarbyl group, e.g. a C₃-C₈ hydrocarbyl group comprising a C₃-C₆ hydrocarbyl ring; the hydrocarbyl group may be saturated, for example exemplary N-substituents are cyclopropyl, cyclobutyl, cyclopentyl and cyclohexyl. As a hydrocarbyl group containing one or more unsaturated bonds may be mentioned phenyl and methyl or ethyl substituted by phenyl, e.g. 2-phenylethyl, as well as β,β-dialkylphenylethyl.

A basis of an aspect of the disclosure is the provision of organoboronic acid products having unexpectedly favourable bioavailability. In this regard, the benefits of the present disclosure include a solution to the problem of boronate diol ester and especially TRI 50b instability, that is to say the presently disclosed products provide inter alia pharmacologically active compounds which are more stable than TRI 50b and other comparable esters in the sense of stability to hydrolysis. The disclosure further includes a solution to the problem of organoboronic acid instability, that is to say the presently disclosed products provide inter alia pharmacologically active compounds which are more stable to deboronation than TRI 50c. The stability provided within the framework of the disclosure is not absolute but is improved relative to the comparator compounds. The benefits offered by the disclosure further include the provision of unexpected products which, contrary to expectation, have a particularly low variability in oral bioavailability.

The Examples in this disclosure contain data showing that the calcium salt of TRI 50c is markedly less soluble than the potassium salt and yet has higher oral bioavailability and higher consistency of oral bioavailability. The finding of an inverse relationship between solubility and bioavailability of two salts is particularly unpredictable. There is no known property of organoboronic acid drugs which accounts for this finding. The disclosure therefore includes amongst other subject matter a TRI 50c derivative which enhances stability as compared with TRI 50b and reduces the variability in absorption which has been observed with TRI 50b and TRI 50c, and advantageously enables adequately consistent and high bioavailability.

The Examples in this disclosure also contain data demonstrating that the calcium salt of TRI 50c is markedly more stable than TRI 50c. Again, there is no known property which accounts for this finding.

The families of compounds represented by formulae (II) and (III) herein , e.g. formula (IIIA), represent near neighbours of TRI 50c which can be predicted to have particularly similar properties to TRI 50c.

Calcium is a representative of a class of pharmaceutically acceptable multivalent metals. It is also a representative of a class of pharmaceutically acceptable divalent metals; as other members of the class may be mentioned magnesium and zinc.

TRI 50c is distinguished from most other organic acid drugs in that the acid group of TRI 50c is a boronic acid and not a carboxylic acid. The data in this disclosure are indicative of multivalent metal salts of organoboronic acid drugs providing a technical effect, not linked to solubility, which enhances the amount and consistency of bioavailability. It does not follow that, because the effect is not linked to solubility, there will in every individual case be for that acid a quantitative relationship between solubility and bioavailability like that observed for TRI 50c.

The disclosure therefore includes oral pharmaceutical formulations comprising a salt of a pharmaceutically acceptable multivalent metal and an organoboronic acid drug. The metal is a Group II or Group III metal or zinc. In a class of formulations the metal is divalent; in one sub-class it is calcium; in another sub-class it is magnesium; in a third sub-class it is zinc. Of course, multivalent metal salts may be used also in parenteral, e.g. intravenous formulations.

There is a debate in the literature as to whether boronates in aqueous solution form the ‘trigonal’ B(OH)₂ or ‘tetrahedral’ B(OH)₃— boron species, but NMR evidence seems to indicate that at a pH below the first pKa of the boronic acid the main boron species is the neutral B(OH)₂. In the duodenum the pH is likely to be between 6 and 7, so the trigonal species is likely to be predominant here. In any event, the symbol —B(OH)₂ includes tetrahedral as well as trigonal boron species, and throughout this specification symbols indicating trigonal boron species embrace also tetrahedral species. The symbol may further include boronic groups in anhydride form.

The salts may be in the form of solvates, particularly hydrates.

The salts may comprise, or consist essentially of, acid salts in which the boronic acid is singly deprotonated. The disclosure therefore includes products having a metal/boronate stoichiometry consistent with the boronate groups in the product predominantly (more than 50 mol %) carrying a single negative charge.

The salts may be in isolated form. The salts may have a purity, e.g. as determined by the method of Example 34, of at least about 90%, e.g. of greater than or equal to about 95%. In the case of pharmaceutical formulations, such salt forms may be combined with pharmaceutically acceptable diluents, excipients or carriers.

Parenteral formulations of the salts are also provided herein. In particular, there are provided parenteral formulations comprising the salts in the solid phase, for example particulate salts for reconstitution as aqueous solutions prior to administration by injection or infusion. Such reconstituted solutions are also included in the disclosure.

According to a further aspect of the present disclosure, there is provided a method of treatment of a condition where anti-thrombotic activity is required which method comprises parenteral administration of a therapeutically effective amount of a pharmaceutically acceptable base addition salt of a boronic acid of formula (I) to a person suffering from, or at risk of suffering from, such a condition.

The disclosure includes subject matter relating to Synthetic Methods I, including a method for preparing the salts from the corresponding boronic acid as an intermediate, as well as the intermediate boronic acid of Formula (I) and a method for preparing it.

An aspect of the disclosure resides in a class of tripeptide boronates useful for making salts described herein and having (R,S,R) stereochemistry. Accordingly, there is provided an isolated compound selected from boronic acids of formula (IIIa): 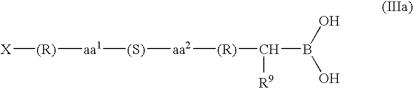
where:

X is H (to form NH₂) or an amino-protecting group;

aa¹ is an amino acid having a hydrocarbyl side chain containing no more than 20 carbon atoms and comprising at least one cyclic group having up to 13 carbon atoms;

aa² is an imino acid having from 4 to 6 ring members;

R⁹ is a straight chain alkyl group interrupted by one or more ether linkages and in which the total number of oxygen and carbon atoms is 3, 4, 5 or 6 or R⁹ is —(CH₂)ₘ—W where m is from 2, 3, 4 or 5 and W is —OH or halogen (F, Cl, Br or I).

A further aspect resides in a process for making a pharmaceutically acceptable base addition salt of an organoboronic acid drug, e.g. an inhibitor of thrombin having a neutral thrombin Si-binding moiety linked through a peptide linkage to a hydrophobic thrombin S2/S3-binding moiety, comprising combining a solution of the organoboronic acid in a water-miscible organic solvent with an aqueous solution or suspension of the base, causing or allowing the acid and the base to react, and recovering the salt.

Additionally included is a solution whose solvent is a water-miscible organic solvent and which contains a boronate drug, e.g. species selected from an organoboronic acid inhibitor of thrombin having a neutral thrombin S1-binding moiety linked through a peptide linkage to a hydrophobic thrombin S2/S3-binding moiety, and equilibrium forms of the organoboronic acid, and combinations thereof, the term “equilibrium foms” meaning differing forms of the same organoboronic acid which may be represented in an equilibrium equation (as in the organoboronic acid in equilibrium with an organoboronic anhydride and in equilibrium with different organoboronate ions).

A further aspect of the disclosure resides in a method of storing an organoboronic acid drug for a period of at least six months, comprising providing the acid in the form of a reaction product thereof with a pharmaceutically acceptable base in a sealed container and storing it for at least six months at a temperature of at least 0° C.

Another disclosed method is a method of storing an organoboronic acid drug for a period of at least six months, comprising providing the acid in the form of a reaction product thereof with a pharmaceutically acceptable base in a sealed container and storing it for at least six months at a temperature of at least 0° C.

A product of the disclosure comprises a package comprising:
(i) a sealed container containing a boronic acid drug in the form of a reaction product thereof with a pharmaceutically acceptable base; and
(ii) instructions permitting the container to be stored at a temperature of 10° C. or more for a period of 8 months or more, e.g. at a temperature of 15° C. or more for a period of 12 months or more.

The organoboronic acid drug referred to in any of the four previous paragraphs may for example be of formula (I), (II) or (III), or of formula (XXX), or by way of example any organoboronic acid drug mentioned in this specification, whether directly or by reference to a publication disclosing it.

2. Synthetic Methods II

TRI 50c salts are obtained via TRI 50c esters. However, published synthetic routes to TRI 50c esters and thus to TRI 50c give rise to one or more impurities. Synthetic Methods I (unpublished as of filing this application) for making the salts give rise to one or more impurities and very high purity salts were not obtained. Further, the salts have proved most challenging to obtain in high purity. Thus, purification techniques which were applied failed to produce very high purity salts. HPLC will not be usable on an industrial scale to purify salts made via published TRI 50c ester syntheses and the salt preparation techniques of Synthetic Methods I. In other words, in order for the therapeutic benefits of TRI 50c salts to be provided to those in need thereof, the salts must be obtainable industrially in adequately pure form and the pure form must be attainable without the use of excessively expensive purification techniques.

The disclosure provides techniques for purifying organoboronic compounds and techniques for helping to maintain the purity of organoboronic compounds, and the products of such techniques. The present disclosure further provides a method of making such high purity salts and the high purity salts themselves. In particular, disclosed herein in one embodiment is a method comprising a chirally-selective precipitation step which results in a precipitated boronic acid derivative of high purity. Further provided is a method for hydrolysing organoboronate that can be used to help obtain high purity salts. In another embodiment, there is disclosed a method for preparing the salts described in the previous paragraph in high purity and wherein selected solvents are used to help achieve high purity levels.

In another aspect there is provided a novel synthesis useful in the preparation of the TRI 50c boropeptide and other compounds; also provided are aminoboronates and boropeptides obtainable indirectly from the synthesis.

There are further provided boronic acid salts of specified purity and pharmaceutical formulations containing them.

In one aspect, the disclosure provides the use of diethanolamine to resolve by precipitation boronic acid compounds (whether provided as the acid or, for example, an ester), wherein the acid is of the formula X—(R)-aa¹-(S)-aa²-NH—C*(R¹)H—B(OH)₂, where aa¹, aa² and R¹ are as described below and C* is a chiral centre present initially in both chiralities. The disclosure further provides a method of resolving the chiral isomers, in which the diethanolamine is used in an amount of 1.25±0.1 equivalents per equivalent of the boronic acid compound having chiral centre C* in (R)-configuration.

Another aspect of the disclosure relates to the protection of organoboronic compounds from degradation by C—B bond cleavage, using a technique not designed to be protective against the previously known oxidative mechanism of C—B bond cleavage. The method comprises the aqueous hydrolysis of a boronic compound, e.g. boronic ester, for a period sufficiently short substantially to avoid cleavage of the C—B bond. By way of example, a period of no more than about 30 minutes at about room temperature may be mentioned.

Further included is the use of acetonitrile as a solvent in the preparation of organoboronate salts. In particular, an organoboronic acid is dissolved in acetonitrile and contacted with a base to form the corresponding organoboronate salt. A solid organoboronate salt containing water may be dried by azeodrying using acetonitrile.

Also provided is a process for separating diastereomers of a boronic acid of formula (XX): 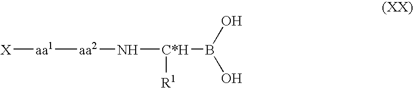
where:
X is H (to form NH₂) or an amino-protecting group;
aa¹ is an amino acid residue of (R) configuration selected from Phe, Dpa and wholly or partially hydrogenated analogues thereof;
aa² is an imino acid residue of (S) configuration having from 4 to 6 ring members;
R¹ is a group of the formula —CH₂)ₛ-Z, where s is 2, 3 or 4 and Z is —OH, —OMe, —OEt or halogen (F, Cl, Br or I),
and where C* is a chiral centre,

the process comprising:
combining (A) a starting solution in diethylether of a boronic species selected from the boronic acid (I) and its esters with alcohols selected from alcohols in which the sole potential electron donor heteroatoms are oxygens which, in the boronic ester, correspond to the oxygens of the ester functional group, the starting solution containing both boronic species having a chiral centre C* of (R) configuration and boronic species having a chiral centre C* of (S) configuration; and (B) diethanolamine, the diethanolamine being in an amount of 1.25±0.1 equivalents based on the boronic species in which chiral centre C* is of (R) configuration, and mixing to form a mixture;
causing or allowing the boronic species and the diethanolamine to react until a precipitate forms; and
recovering the precipitate.

The precipitation step is selective for species having a chiral centre C* of (R) configuration, which are recovered in high purity.

The process may comprise converting the recovered precipitate to the acid of formula (I) by dissolving the precipitate in an organic solvent selected from halohydrocarbons and combinations thereof, agitating the resulting solution with an aqueous medium, for example an aqueous acid having a pH of below 3, whereby the dissolved precipitate is converted to the formula (I) acid, and recovering the formula (I) acid by evaporation.

One process of the disclosure comprises hydrolysing, e.g. allowing the hydrolysis of, a diethanolamine ester of an acid of formula (I) with an aqueous medium for a time sufficiently short for the product acid to be substantially free of impurity resulting from carbon-boron bond cleavage.

One class of processes further comprises converting the recovered acid of formula (I) to a pharmaceutically acceptable base addition salt thereof by dissolving the acid in acetonitrile, combining the resultant solution with an aqueous solution or suspension of a pharmaceutically acceptable base, and causing or allowing the base and the acid to react, then evaporating to dryness to obtain an evaporation residue. In more general terms, a boronic acid drug in acetonitrile solution may be combined with an aqueous solution or suspension of a base in this way, to form a reaction product useful for incorporating in a pharmaceutical formulation.

The reaction product may therefore be incorporated in a pharmaceutical formulation.

The invention further includes a process for making a boronic acid of Formula (I) in which R¹ is of the formula —(CH₂)ₛ—O—R³ wherein R³ is methyl or ethyl and s is independently 2, 3 or 4, or for making a synthetic intermediate for such an acid, the process comprising:
reacting a 1-metalloalkoxyalkane, where the alkoxyalkane is of the formula —(CH₂)ₛ—O—R³, and a borate ester to form a compound of Formula (VI):
   (HO)₂B—(CH₂)ₛ—O—R³ (VI),
   the process optionally further comprising converting the compound of Formula (VI) into an acid of formula (I), for example by a known process.

In one class of processes, the compound of Formula (VI) is converted into an ester of the Formula (I) acid, which ester is transesterified with diethanolamine to form a precipitate. The precipitate may then be recovered for further processing. Suitably, the diethanolamine transesterification is used for resolving chiral isomers, as described herein. The resolved active R,S,R isomer may then be converted from the diethanolamine ester to the free acid, for example as described herein, and the free acid may if desired be converted to a salt, for example as described herein.

The disclosure includes the products of the aforesaid processes. Further products are described and claimed in the following specification.

The Synthetic Methods II and products thereof may be performed or, as the case may be, provided on mass or commercial scale.

3. General

The salts described herein include products obtainable by (having the characteristics of a product obtained by) reaction of the boronic acid with a strong base and the term “salt” herein is to be understood accordingly. The term “salt” in relation to the disclosed products, therefore, does not necessarily imply that the products contain discrete cations and anions and is to be understood as embracing products which are obtainable using a reaction of a boronic acid and a base. The disclosure embraces products which, to a greater or lesser extent, are in the form of a coordination compound. The disclosure thus provides also products obtainable by (having the characteristics of a product obtained by) reaction of a boronic acid drug, e.g. of Formula (I) or Formula (XXX) with a strong base a well as the therapeutic, including prophylactic, use of such products.

The present disclosure is not limited as to the method of preparation of the salts, provided that they contain a boronate species derived from boronic acid drug and a counter-ion. Such boronate species may be boronate anions in any equilibrium form thereof. The term “equilibrium form” refers to differing forms of the same compounds which may be represented in an equilibrium equation (e.g. boronic acid in equilibrium with a boronic anhydride and in equilibrium with different boronate ions). Boronates in the solid phase may form anhydrides and the disclosed boronate salts when in the solid phase may comprise boronate anhydrides, as a boronic equilibrium species. It is not required that the salts be prepared by reaction of a base containing the counter-ion and the boronic acid. ° Further, the disclosure includes salt products which might be regarded as indirectly prepared by such an acid/base reaction as well as salts obtainable by (having the characteristics of products obtained by) such indirect preparation. As examples of possibly indirect preparation may be mentioned processes in which, after initial recovery of the salt, it is purified and/or treated to modify its physicochemical properties, for example to modify solid form or hydrate form, or both.

In some embodiments, the cations of the salts are monovalent.

In some embodiments the salts comprise anhydride species; in others they are essentially free of anhydride species.

Further aspects and embodiments of the disclosure are set forth in the following description and claims. Also included as such are the salts described herein.

Throughout the description and claims of this specification, the words “comprise” and “contain” and variations of the words, for example “comprising” and “comprises”, mean “including but not limited to”, and are not intended to (and do not) exclude other moieties, additives, components, integers or steps.

This patent application contains data indicating that the stability (resistance to deboronation) of organoboronic acids may be increased by providing them in the form of salts, e.g. metal salts. In single experiments, the ammonium salt of TRI 50c appeared to decompose on drying to yield ammonia, whilst the choline salt demonstrated rapid decomposition to a deboronated impurity. Although experiments have not been conducted to reproduce these unrepeated observations, there is provided a sub-class in which the ammonium and choline salts are excluded. The salt may be an acid salt. In any event, this stabilisation technique forms part of the disclosure and is applicable, inter alia, to organoboronic acids described under the heading “BACKGROUND” and to organoboronic acids described in publications mentioned under that heading.

### BRIEF DESCRIPTION OF THE DRAWINGS

FIG. 1 is a chart referred to in Example 35, showing the results of a thrombin amidolytic assay of TRI 1405 (TRI 50c magnesium salt) and TRI 50b, where Vmax is the maximum rate of reaction measured by amidolytic assay.

FIG. 2 is an HPLC plot referred to in Example 41, showing an impurity profile of encapsulated TRI 50c calcium salt after having been maintained in blister packaging for 1.5 month at 25° C. and 60% relative humidity.

FIG. 3 is an HPLC plot referred to in Example 41, showing an impurity profile of encapsulated TRI 50c calcium salt after having been maintained in blister packaging for 1.5 month at 40° C. and 75% relative humidity.

FIG. 4 is an HPLC plot referred to in Example 41, showing an impurity profile of encapsulated TRI 50c calcium salt after having been maintained absent blister packaging for 1.5 month at 40° C. and 75% relative humidity.

### DETAILED DESCRIPTION OF SEVERAL EXAMPLES

Glossary

The following terms and abbreviations are used in this specification:

The expression “acid salt” as applied to a salt of a boronic acid refers to salts of which a single —OH group of the trigonally-represented acid group —B(OH)₂ is deprotonated. Thus salts wherein the boronate group carries a single negative charge and may be represented as —B(OH)(O⁻) or as [—B(OH)₃]⁻ are acid salts. The expression encompasses salts of a cation having a valency n wherein the molar ratio of boronic acid to cation is approximately n to 1. In practical terms, the observed stoichiometry is unlikely to be exactly n:1 but will be consistent with a notional n:1 stoichiometry. For example, the observed mass of the cation might vary from the calculated mass for a n:1 stoichiometry by no more than about 10%, e.g. no more than about 7.5%; in some cases an observed mass of a cation might vary from the calculated mass by no more than about 1%. Calculated masses are suitably based on the trigonal form of the boronate. (At an atomic level, a salt stoichiometrically consistent with being an acid salt might contain boronates in a mix of protonation states, whose average approximates to single deprotonation and such “mixed” salts are included in the term “acid salt”). Examples of acid salts are monosodium salts and hemicalcium salts.

α-Aminoboronic acid or Boro(aa) refers to an amino acid in which the CO₂ group has been replaced by BO₂.

The term “amino-group protecting moiety” refers to any group used to derivatise an amino group, especially an N-terminal amino group of a peptide or amino acid. Such groups include, without limitation, alkyl, acyl, alkoxycarbonyl, aminocarbonyl, and sulfonyl moieties. However, the term “amino-group protecting moiety” is not intended to be limited to those particular protecting groups that are commonly employed in organic synthesis, nor is it intended to be limited to groups that are readily cleavable.

The term “equilibrium form” refers to differing forms of the same compounds which may be represented in an equilibrium equation, as in the case of a boronic acid in equilibrium with a boronic anhydride and/or in equilibrium with one or more different boronate ions or as in the case of an organic base in equilibrium with a protonated form thereof.

The phrase “pharmaceutically acceptable” is employed herein to refer to those compounds, materials, compositions, and/or dosage forms which are, within the scope of sound medical judgment, suitable for use, in contact with the tissues of human beings or animals without excessive toxicity, irritation, allergic response, or other problem or complication, commensurate with a reasonable benefit/risk ratio.

The expression “thrombin inhibitor” refers to a product which, within the scope of sound pharmacological judgement, is potentially or actually pharmaceutically useful as an inhibitor of thrombin, and includes reference to substance which comprises a pharmaceutically active species and is described, promoted or authorised as a thrombin inhibitor. Such thrombin inhibitors may be selective, that is they are regarded, within the scope of sound pharmacological judgement, as selective towards thrombin in contrast to other proteases; the term “selective thrombin inhibitor” includes reference to substance which comprises a pharmaceutically active species and is described, promoted or authorised as a selective thrombin inhibitor.

The expression “proteasome inhibitor” refers to a product which, within the scope of sound pharmacological judgement, is potentially or actually pharmaceutically useful as an inhibitor of proteasome, and includes reference to substance which comprises a pharmaceutically active species and is described, promoted or authorised as a proteasome inhibitor. Such proteasome inhibitors may be selective, that is they are regarded, within the scope of sound pharmacological judgement, as selective towards proteasome in contrast to other proteases; the term “selective proteasome inhibitor” includes reference to substance which comprises a pharmaceutically active species and is described, promoted or authorised as a selective proteasome inhibitor.

The term “heteroaryl” refers to a ring system which has at least one (e.g. 1, 2 or 3) in-ring heteroatoms and has a conjugated in-ring double bond system. The term “heteroatom” includes oxygen, sulfur and nitrogen, of which sulfur is sometimes less preferred.

“Natural amino acid” means an L-amino acid (or residue thereof) selected from the following group of neutral (hydrophobic or polar), positively charged and negatively charged amino acids:

Hydrophobic Amino Acids

A=Ala=alanine

V=Val=valine

I=Ile=isoleucine

L=Leu=leucine

M=Met=methionine

F=Phe=phenylalanine

P=Pro=proline

W=Trp=tryptophan

Polar (Neutral or Uncharged) Amino Acids

N=Asn=asparagine

C=Cys=cysteine

Q=Gin=glutamine

G=Gly=glycine

S=Ser=serine

T=Thr=threonine

Y=Tyr=tyrosine

Positively Charged (Basic) Amino Acids

R=Arg=arginine

H=His=histidine

K=Lys=lysine

Negatively Charaed Amino Acids

D=Asp=aspartic acid

E=Glu=glutamic acid.

ACN=acetonitrile

Amino acid=α-amino acid

Base addition salt=a salt which is prepared from addition of an inorganic base or an organic base to a free acid (in this case the boronic acid).

Cbz=benzyloxycarbonyl

Cha=cyclohexylalanine (a hydrophobic unnatural amino acid)

Charged (as applied to drugs or fragments of drug molecules, e.g. amino acid residues)=carrying a charge at physiological pH, as in the case of an amino, amidino or carboxy group

Dcha=dicyclohexylalanine (a hydrophobic unnatural amino acid)

Dpa=diphenylalanine (a hydrophobic unnatural amino acid)

Drug=a pharmaceutically useful substance, whether the active in vivo principle or a prodrug

i.v.=intravenous

Mpg=3-methoxypropylglycine (a hydrophobic unnatural amino acid)

Multivalent=valency of at least two, for example two or three

Neutral (as applied to drugs or fragments of drug molecules, e.g. amino acid residues)=uncharged=not carrying a charge at physiological pH

Pinac=Pinacol=2,3-dimethyl-2,3-butanediol

Pinanediol=2,3-pinanediol=2,6,6-trimethylbicyclo [3.1.1] heptane-2,3-diol

Pip=pipecolinic acid

Room temperature=25° C.±2° C.

s.c.=subcutaneous

Strong base=a base having a sufficiently high pKb to react with a boronic acid. Suitably such bases have a pKb of 7 or more, e.g. 7.5 or more, for example about 8 or more

THF=tetrahydrofuran

Thr=thrombin

Novel Products—The Compounds

The products of the disclosure comprise base addition salts of boronic acid drugs. One class of such drugs have the formula (XXX): 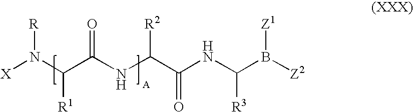
wherein:
X is hydrogen or an amino-group protecting moiety;
R is hydrogen or alkyl;
A is 0, 1 or 2;
R¹, R² and R³ are independently hydrogen, alkyl, cycloalkyl, aryl or —CH₂—R⁵;
R⁵, in each instance, is one of aryl, aralkyl, alkaryl, cycloalkyl, heterocyclyl, heteroaryl, or —W—R⁶, where W is a chalcogen and R⁶ is alkyl;
where the ring portion of any of said aryl, aralkyl, alkaryl, cycloalkyl, heterocyclyl, or heteroaryl in R¹, R², R³ or R⁵ can be optionally substituted; and
Z¹ and Z² together form a moiety derived from a sugar, wherein the atom attached to boron in each case is an oxygen atom.

The compounds of formula (XXX) are useful as proteasome inhibitors.

X is a moiety bonded to the N-terminal amino group and may be H. The identity of X is not critical but may be a particular X moiety described above. In one example relating to the compounds of Formula (XXX) there may be mentioned (2-pyrazine)carbonyl; another example is (2-pyrazine)sulfonyl

In certain examples X is R⁶—(CH₂)ₚ—C(O)—, R⁶—(CH₂)ₚ—S(O)₂—, R⁶—(CH₂)ₚ—NH—C(O)— or R⁶—(CH₂)ₚ—O—C(O)— wherein p is 0, 1, 2, 3, 4, 5 or 6 (of which 0 and 1 are preferred) and R⁶ is H or a 5 to 13-membered cyclic group optionally substituted by 1, 2 or 3 substituents selected from halogen, amino, nitro, hydroxy, a C₅-C₆ cyclic group, C₁-C₄ alkyl and C₁-C₄ alkyl containing, and/or linked to the 5 to 13-membered cyclic group through, an in-chain O, the aforesaid alkyl groups optionally being substituted by a substituent selected from halogen, amino, nitro, hydroxy and a C₅-C₆ cyclic group. More particularly X is R⁶—(CH₂)ₚ—C(O)— or R⁶—(CH₂)ₚ—O—C(O)— and p is 0 or 1. Said 5 to 13-membered cyclic group is often aromatic or heteroaromatic, for example is a 6-membered aromatic or heteroaromatic group. In many cases, the group is not substituted.

Exemplary X groups are (2-pyrazine)carbonyl, (2-pyrazine)sulfonyl and particularly benzyloxy-carbonyl.

In embodiments of the compounds of Formula (XXX), the compounds contain 1 or any combination of the following features (e.g. all of them):
A is zero
R is hydrogen or 1C-8C alkyl
R³ is 1C-6C alkyl.

In a class of Formula (XXX) compounds, R¹, R², and R³ are each independently one of hydrogen, 1C-8C alkyl, 3C-10C cycloalkyl or 6C-10C aryl, or —CH₂—R⁵; and R⁵, in each instance, is one of 6C-10C aryl, (6C-10C)ar(1C-6C)alkyl, (1C-6C)alk(6C-10C)aryl, 3C-20C cycloalkyl, 1C-8C alkoxy or 1C-8C alkylthio, where the ring portion of any of said aryl, aralkyl, alkaryl and cycloalkyl groups of R¹, R², R³ or R⁵ is optionally substituted.

With reference to the compounds of Formula (XXX), the meanings of the following terms are to be noted:

The term “alkyl” refers to straight and branched chain aliphatic groups having from 1 to 12 carbon atoms, preferably 1-8 carbon atoms, more preferably 1-6 carbon atoms, and still more preferably 1-4 carbon atoms, which may be optionally substituted with one, two or three substituents. Unless otherwise explicitly stated, the term “alkyl” is meant to include saturated, unsaturated, and partially unsaturated aliphatic groups. When unsaturated groups are particularly intended, the terms “alkenyl” or “alkynyl” will be used. When only saturated groups are intended, the term “saturated alkyl” will be used. Preferred saturated alkyl groups include, without limitation, methyl, ethyl, propyl, isopropyl, butyl, isobutyl, sec-butyl, tert-butyl, pentyl, and hexyl. In all contexts, a particular class of alkyl groups are saturated alkyl groups.

The term “cycloalkyl” includes saturated and partially unsaturated cyclic hydrocarbon groups having 3 to 12 carbons, preferably 3 to 8 carbons, and more preferably 3 to 6 carbons, wherein the cycloalkyl group additionally may be optionally substituted. Preferred cycloalkyl groups include, without limitation, cyclopropyl, cyclobutyl, cyclopentyl, cyclopentenyl, cyclohexyl, cyclohexenyl, cycloheptyl, and cyclooctyl.

An “aryl” group is a 6C-14C aromatic moiety comprising one to three aromatic rings, which may be optionally substituted. Preferably, the aryl group is a 6C-10C aryl group. Preferred aryl groups include, without limitation, phenyl, naphthyl, anthracenyl, and fluorenyl. An “aralkyl” or “arylalkyl” group comprises an aryl group covalently linked to an alkyl group, either of which may independently be optionally substituted or unsubstituted. Preferably, the aralkyl group is (1C-6C)alk(6C-10C)aryl, including, without limitation, benzyl, phenethyl, and naphthylmethyl. An “alkaryl” or “alkylaryl” group is an aryl group having one or more alkyl substituents. Examples of alkaryl groups include, without limitation, tolyl, xylyl, mesityl, ethylphenyl, tert-butylphenyl, and methyinaphthyl.

The terms “heterocycle”, “heterocyclic”, and “heterocyclyl” refer to any stable ring structure having from about 3 to about 8 atoms, wherein one or more atoms are selected from the group consisting of N, O, and S. The nitrogen and sulfur heteroatoms of the heterocyclic moiety may be optionally oxidized, and the nitrogen atoms may be optionally quaternized. The heterocyclic ring can be attached to its pendant group at any heteroatom or carbon atom that results in a stable formula. The term “stable compound” or “stable formula” is meant to refer to a compound that is sufficiently robust to survive isolation to a useful degree of purity from a reaction mixture and formulation into an efficacious therapeutic agent.

The heterocyclic group may be optionally substituted on carbon at one or more positions with any of the substituents recited above. The heterocyclic group may also independently be substituted on nitrogen with alkyl, aryl, aralkyl, alkylcarbonyl, alkylsulfonyl, arylcarbonyl, arylsulfonyl, alkoxycarbonyl, aralkoxycarbonyl, oxo, or hydroxy, or on sulfur with oxo or lower alkyl. Preferred heterocyclic groups include, without limitation, epoxy, aziridinyl, tetrahydrofuranyl, pyrrolidinyl, piperidinyl, piperazinyl, thiazolidinyl, oxazolidinyl, oxazolidinonyl, and morpholinyl. The heterocyclic group may also be fused to an aryl, heteroaryl, or heterocyclic group. Examples of such fused heterocyles include, without limitation, tetrahydroquinoline and dihydrobenzofuran.

The terms “heteroaryl” and “aromatic heterocyle” refer to groups having 5 to 14 ring atoms, preferably 5, 6, 9, or 10 ring atoms; having 6, 10, or 14 .pi. electrons shared in a cyclic array; and having, in addition to carbon atoms, from one to about four, preferably from one to about three, heteroatoms selected from the group consisting of N, O, and S. The heteroaryl group may be optionally substituted on carbon at one or more positions with any of the substituents recited above. Preferred heteroaryl groups include, without limitation, thienyl, benzothienyl, furyl, benzofuryl, dibenzofuryl, pyrrolyl, imidazolyl, pyrazolyl, pyridyl, pyrazinyl, pyrimidinyl, indolyl, quinolyl, isoquinolyl, quinoxalinyl, tetrazolyl, oxazolyl, thiazolyl, and isoxazolyl.

A “substituted” alkyl, cycloalkyl, aryl, heterocyclyl, or heteroaryl group is one having from one and to about four, preferably from one to about three, more preferably one or two, non-hydrogen substituents. Suitable substituents include, without limitation, halo, hydroxy, oxo, nitro, haloalkyl, alkyl, alkaryl, aryl, aralkyl, alkoxy, aryloxy, amino, acylamino, alkylcarbamoyl, arylcarbamoyl, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, acyloxy, cyano, and ureido groups. Preferably the substituents are independently selected from the group consisting of 1C-6C alkyl, 3C-8C cycloalkyl, (1C-6C)alkyl(3C-8C)cycloalkyl, 2C-8C alkenyl, 2C-8C alkynyl, cyano, amino, 1C-6C alkylamino, di(1C-6C)alkylamino, benzylamino, dibenzylamino, nitro, carboxy, carbo(1C-6C)alkoxy, trifluoromethyl, halogen, 1C-6C alkoxy, 6C-10C aryl, (6C-10C)aryl(1C-6C)alkyl, (6C-10C)aryl(1C-6C)alkoxy, hydroxy, 1C-6C alkylthio, 1C-6C alkylsulfinyl, 1C-6C alkylsulfonyl, 6C-10C arylthio, 6C-10C arylsulfinyl, 6C-10C arylsulfonyl, 6C-10C aryl, (1C-6C)alkyl(6C-10C)aryl, and halo(6C-10C)aryl.

The term “halogen” or “halo” as employed herein refers to chlorine, bromine, fluorine, or iodine.

The term oxo refers to an oxygen atom, which forms a carbonyl when attached to carbon, an N-oxide when attached to nitrogen, and a sulfoxide or sulfone when attached to sulfur.

As herein employed, the term “acyl” refers to an alkylcarbonyl or arylcarbonyl substituent

The term “acylamino” refers to an amide group attached at the nitrogen atom. The term “carbamoyl” refers to an amide group attached at the carbonyl carbon atom. The nitrogen atom of an acylamino or carbamoyl substituent may be additionally substituted. The term “sulfonamido” refers to a sulfonamide substituent attached by either the sulfur or the nitrogen atom. The term “amino” is meant to include NH₂, alkylamino, arylamino, and cyclic amino groups.

The term “ureido” as employed herein refers to a substituted or unsubstituted urea moiety

Exemplary compounds of Formula (XXX) are:

N-(2-pyrazine)carbonyl-L-phenylalanine-L-leucine boronate;

N-(2-quinoline)sulfonyl-L-homophenylalanine-L-leucine boronate;

N-(3-pyridine)carbonyl-L-phenylalanine-L-leucine boronate;

N-(4-morpholine)carbonyl-L-phenylalanine-L-leucine boronate;

N-(4-morpholine)carbonyl-.beta.-(1-naphthyl)-L-alanine-L-leucine boronate;

N-(8-quinoline)sulfonyl-.beta.-(1-naphthyl)-L-alanine-L-leucine boronate

N-(4-morpholine)carbonyl-(O-benzyl)-L-tyrosine-L-leucine boronate;

N-(4-morpholine)carbonyl-L-tyrosine-L-leucine boronate; or

N-(4-morpholine)carbonyl-[O-(2-pyridylmethyl)]-L-tyrosine-L-leucine boronate.

For more information concerning compounds of formula (XXX), the reader is referred to U.S. Pat. Nos. 6,617,317; 6,548,668; 6,465,433; 6,297,217; 6,066,730; 5,780,454; and 6,083,903, all of which are incorporated herein by reference in their entirety.

The products of the disclosure further comprise salts of boronic acids which have a neutral aminoboronic acid residue capable of binding to the thrombin S1 subsite linked through a peptide linkage to a hydrophobic moiety capable of binding to the thrombin S2 and S3 subsites.

The disclosure includes salts of acids of formula (I): 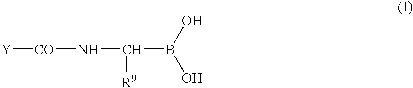
wherein
Y comprises a hydrophobic moiety which, together with the aminoboronic acid residue —NHCH(R⁹)—B(OH)₂, has affinity for the substrate binding site of thrombin; and

R⁹ is a straight chain alkyl group interrupted by one or more ether linkages and in which the total number of oxygen and carbon atoms is 3, 4, 5 or 6 (e.g. 5) or R⁹ is —CH₂)ₘ—W where m is from 2, 3, 4 or 5 (e.g. 4) and W is —OH or halogen (F, Cl, Br or I). As examples of straight chain alkyl interrupted by one or more ether linkages (—O—) may be mentioned alkoxyalkyl (one interruption) and alkoxyalkoxyalkyl (two interruptions). R⁹ is an alkoxyalkyl group in one subset of compounds, e.g. alkoxyalkyl containing 4 carbon atoms.

Typically, YCO— comprises an amino acid residue (whether natural or unnatural) which binds to the S2 subsite of thrombin, the amino acid residue being N-terminally linked to a moiety which binds the S3 subsite of thrombin.

In one class of Formula (I) acids, YCO— is an optionally N-terminally protected dipeptide residue which binds to the S3 and S2 binding sites of thrombin and the peptide linkages in the acid are optionally and independently N-substituted by a C₁-C₁₃ hydrocarbyl group optionally containing in-chain and/or in-ring nitrogen, oxygen or sulfur and optionally substituted by a substituent selected from halo, hydroxy and trifluoromethyl. The N-terminal protecting group, when present, may be a group X as defined above (other than hydrogen). Normally, the acid contains no N-substituted peptide linkages; where there is an N-substituted peptide linkage, the substituent is often 1C to 6C hydrocarbyl, e.g. saturated hydrocarbyl; the N-substituent comprises a ring in some embodiments, e.g. cycloalkyl, and may be cyclopentyl, for example. One class of acids has an N-terminal protecting group (e.g. an X group) and unsubstituted peptide linkages.

Where YCO— is a dipeptide residue (whether or not N-terminally protected), the S3-binding amino acid residue may be of R configuration and/or the S2-binding residue may of S configuration. The fragment —NHCH(R⁹)—B(OH) may of R configuration. The disclosure is not restricted to chiral centres of these conformations, however.

In one class of compounds, the side chain of P3 (S3-binding) amino acid and/or the P2 (S2-binding) amino acid is a moiety other than hydrogen selected from a group of formula A or B:
—(CO)ₐ—(CH₂)_{b}-D_{c}-(CH₂)_{d}-E (A)
—(CO)ₐ—(CH₂)_{b}-D_{c}-Cₑ(E¹)(E²)(E³) (B)
wherein

a is 0 or 1;

e is 1;

b and d are independently 0 or an integer such that (b+d) is from 0 to 4 or, as the case may be,

(b+e) is from 1 to 4;

c is 0 or 1;

D is O or S;

E is H, C₁-C₆ alkyl, or a saturated or unsaturated cyclic group which normally contains up to 14 members and particularly is a 5-6 membered ring (e.g. phenyl) or an 8-14 membered fused ring system (e.g. naphthyl), which alkyl or cyclic group is optionally substituted by up to 3 groups (e.g. 1 group) independently selected from C₁-C₆ trialkylsilyl, —CN, —R¹³, —R¹²OR¹³, —R¹²COR¹³, —R¹²CO₂R¹³ and —R¹²O₂CR¹³, wherein R¹² is —(CH₂)_{f}— and R¹³ is —(CH₂)_{g}H or by a moiety whose non-hydrogen atoms consist of carbon atoms and in-ring heteroatoms and number from 5 to 14 and which contains a ring system (e.g. an aryl group) and optionally an alkyl and/or alkylene group, wherein f and g are each independently from 0 to 10, g particularly being at least 1 (although —OH may also be mentioned as a substituent), provided that (f+g) does not exceed 10, more particularly does not exceed 6 and most particularly is 1, 2, 3 or 4, and provided that there is only a single substituent if the substituent is a said moiety containing a ring system, or E is C₁-C₆ trialkylsilyl; and E¹, E² and E³ are each independently selected from —R¹⁵ and -J-R¹⁵, where I is a 5-6 membered ring and R¹⁵ is selected from C₁-C₆ trialkylsilyl, —CN, —R¹³, —R¹²OR¹³, —R¹²COR¹³, —R¹²CO₂R¹³, —R¹²O₂CR¹³, and one or two halogens (e.g. in the latter case to form a -3-R¹⁵ moiety which is dichlorophenyl), where R¹² and R¹³ are, respectively, an R¹² moiety and an R¹³ moiety as defined above (in some acids where E¹, E² and E³ contain an R¹³ group, g is 0 or 1);

in which moiety of Formula (A) or (B) any ring is carbocyclic or aromatic, or both, and any one or more hydrogen atoms bonded to a carbon atom is optionally replaced by halogen, especially F.

In certain examples, a is 0. If a is 1, c may be 0. In particular examples, (a+b+c+d) and (a+b+c+e) are no more than 4 and are more especially 1, 2 or 3. (a+b+c+d) may be 0.

Exemplary groups for E, E¹, E² and E³ include aromatic rings such as phenyl, naphthyl, pyridyl, quinolinyl and furanyl, for example; non-aromatic unsaturated rings, for example cyclohexenyl; saturated rings such as cyclohexyl, for example. E may be a fused ring system containing both aromatic and non-aromatic rings, for example fluorenyl. One class of E, E¹, E² and E³ groups are aromatic (including heteroaromatic) rings, especially 6-membered aromatic rings. In some compounds, E¹ is H whilst E² and E³ are not H; in those compounds, examples of E² and E³ groups are phenyl (substituted or unsubstituted) and C₁-C₄ alkyl, e.g. methyl.

In one class of embodiments, E contains a substituent which is C₁-C6 alkyl, (C₁-C₅ alkyl)carbonyl, carboxy C₁-C₅ alkyl, aryl (including heteroaryl), especially 5-membered or preferably 6-membered aryl (e.g. phenyl or pyridyl), or arylalkyl (e.g. arylmethyl or arylethyl where aryl may be heterocyclic and is preferably 6-membered).

In another class of embodiments, E contains a substituent which is OR¹³, wherein R¹³ can be a 6-membered ring, which may be aromatic (e.g. phenyl) or is alkyl (e.g. methyl or ethyl) substituted by such a 6-membered ring.

A class of moieties of formula A or B are those in which E is a 6-membered aromatic ring optionally substituted, particularly at the 2-position or 4-position, by —R¹³ or —OR¹³.

The disclosure includes salts in which the P3 and/or P2 side chain comprises a cyclic group in which 1 or 2 hydrogens have been replaced by halogen, e.g. F or Cl.

The disclosure includes a class of salts in which the side chains of formula (A) or (B) are of the following formulae (C), (D) or (E): 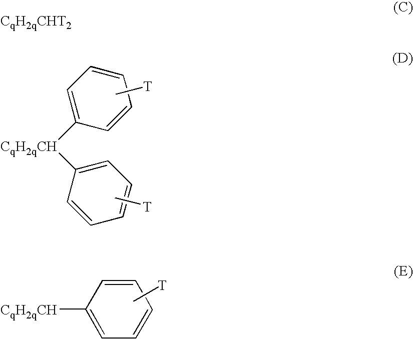
wherein q is from 0 to 5, e.g. is 0, 1 or 2, and each T is independently hydrogen, one or two halogens (e.g. F or Cl), —SiMe₃, —CN, —R¹³, —OR¹³, —COR¹³, —CO₂R¹³ or —O₂CR¹³. In some embodiments of structures (D) and (E), T is at the 4-position of the phenyl group(s) and is —R¹³, —OR¹³, —COR¹³, —CO₂R¹³ or —O₂CR¹³, and R¹³ is C₁-C₁₀ alkyl and more particularly C₁-C₆ alkyl. In one sub-class, T is —R¹³ or —OR¹³, for example in which f and g are each independently 0, 1, 2 or 3; in some side chains groups of this sub-class, T is —R¹²OR¹³ and R¹³ is H.

In one class of the moieties, the side chain is of formula (C) and each T is independently R¹³ or OR¹³ and R¹³ is C₁-C₄ alkyl. In some of these compounds, R¹³ is branched alkyl and in others it is straight chain. In some moieties, the number of carbon atoms is from 1 to 4.

In many dipeptide fragments YCO— (which dipeptides may be N-terminally protected or not), the P3 amino acid has a side chain of formula (A) or (B) as described above and the P2 residue is of an imino acid.

The disclosure therefore includes medicaments comprising salts, e.g. metal salts, of organoboronic acids which are thrombin inhibitors, particularly selective thrombin inhibitors, having a neutral P1 (S1-binding) moiety. For more information about moieties which bind to the 53, S2 and Si sites of thrombin, see for example Tapparelli C et al, Trends Pharmacol. Sci. 14: 366-376, 1993; Sanderson P et al, Current Medicinal Chemistry, 5: 289-304, 1998; Rewinkel J et al, Current Pharmaceutical Design, 5:1043-1075, 1999; and Coburn C Exp. Opin. Ther. Patents 11(5): 721-738, 2001. The thrombin inhibitory salts of the disclosure are not limited to those having S3, S2 and S1 affinity groups described in the publications listed in the preceding sentence.

The boronic acids may have a Ki for thrombin of about 100 nM or less, e.g. about 20 nM or less.

A subset of the Formula (I) acids comprises the acids of Formula (III):

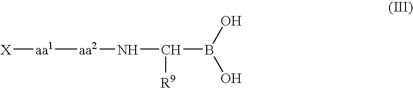

X is a moiety bonded to the N-terminal amino group and may be H to form NH₂. The identity of X is not critical but may be a particular X moiety described above. In one example there may be mentioned benzyloxycarbonyl.

In certain examples X is R⁶—(CH₂)ₚ—C(O)—, R⁶—(CH₂)ₚ—S(O)₂—, R⁶—(CH₂)ₚ—NH—C(O)— or R⁶ is H (CH₂)ₚ—O—C(O)— wherein p is 0, 1, 2, 3, 4, 5 or 6 (of which 0 and 1 are preferred) and R⁶ is H or a 5 to 13-membered cyclic group optionally substituted by 1, 2 or 3 substituents selected from halogen, amino, nitro, hydroxy; a C₅-C₆ cyclic group, C₁-C₄ alkyl and C₁-C₄ alkyl containing, and/or linked to the 5 to 13-membered cyclic group through, an in-chain O, the aforesaid alkyl groups optionally being substituted by a substituent selected from halogen, amino, nitro, hydroxy and a C₅-C₆ cyclic group. More particularly X is R⁶—(CH₂)ₚ—C(O)— or R⁶—(CH₂)ₚ—O—C(O)— and p is 0 or 1. Said 5 to 13-membered cyclic group is often aromatic or heteroaromatic, for example is a 6-membered aromatic or heteroaromatic group. In many cases, the group is not substituted.

Exemplary X groups are (2-pyrazine) carbonyl, (2-pyrazine) sulfonyl and particularly benzyloxycarbonyl.

aa¹ is an amino acid residue having a hydrocarbyl side chain containing no more than 20 carbon atoms (e.g. up to 15 and optionally up to 13 C atoms) and comprising at least one cyclic group having up to 13 carbon atoms. In certain examples, the cyclic group(s) of aa¹ have/has 5 or 6 ring members. For instance, the cyclic group(s) of aa¹ may be aryl groups, particularly phenyl. Typically, there are one or two cyclic groups in the aa¹ side chain. Certain side chains comprise, or consist of, methyl substituted by one or two 5- or 6-membered rings.

More particularly, aa¹ is Phe, Dpa or a wholly or partially hydrogenated analogue thereof. The wholly hydrogenated analogues are Cha and Dcha.

aa² is an imino acid residue having from 4 to 6 ring members. Alternatively, aa² is Gly N-substituted by a C₃-C₁₃ hydrocarbyl group, e.g. a C₃-C₈ hydrocarbyl group comprising a C₃-C₆ hydrocarbyl ring; the hydrocarbyl group may be saturated, for example exemplary N-substituents are cyclopropyl, cyclobutyl, cyclopentyl and cyclohexyl. As a hydrocarbyl group containing one or more unsaturated bonds may be mentioned phenyl and methyl or ethyl substituted by phenyl, e.g. 2-phenylethyl, as well as β,β-dialkylphenylethyl.

An exemplary class of products comprises those in which aa² is a residue of an imino acid of formula (IV) 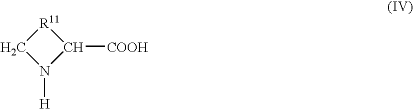
where R¹¹ is —CH₂—, CH₂—CH₂—, —S—CH₂— or —CH₂—CH₂—CH₂—, which group when the ring is 5 or 6-membered is optionally substituted at one or more —CH₂— groups by from 1 to 3 C₁-C₃ alkyl groups, for example to form the R¹¹ group —S—C(CH₃)₂—. Of these imino acids, azetidine-2-carboxylic acid, especially (s)-azetidine-2-carboxylic acid, and more particularly proline are illustrative.

It will be appreciated from the above that a very preferred class of products consists of those in which aa¹-aa² is Phe-Pro. In another preferred class, aa¹-aa² is Dpa-Pro. In other products, aa¹-aa² is Cha-Pro or Dcha-Pro. Of course, also included are corresponding product classes in which Pro is replaced by (s)-azetidine-2-carboxylic acid.

R⁹ is as defined previously and may be a moiety R¹ of the formula ≧(CH₂)ₛ-Z. Integer s is 2, 3 or 4 and W is —OH, -Me, —OEt or halogen (F, Cl, I or, preferably, Br). Particularly illustrative Z groups are —OMe and —OEt, especially —OMe. In certain examples s is 3 for all Z groups and, indeed, for all compounds of the disclosure. Particular R¹ groups are 2-bromoethyl, 2-chloroethyl, 2-methoxyethyl, 4-bromobutyl, 4-chlorobutyl, 4-methoxybutyl and, especially, 3-bromopropyl, 3-chloropropyl and 3-methoxypropyl. Most preferably, R¹ is 3-methoxypropyl. 2-Ethoxyethyl is another preferred R¹ group.

Accordingly, a specific class of salts consists of those of acids of the formula X-Phe-Pro-Mpg-B(OH)₂, especially Cbz-Phe-Pro-Mpg-B(OH)₂; also included are analogues of these compounds in which Mpg is replaced by a residue with another of the R¹ groups and/or Phe is replaced by Dpa or another aa¹ residue. Also included are compounds in which Cbz is replaced by benzylmethylcarbonyl (Ph-Et-CO—).

The aa¹ moiety of the acid is preferably of R configuration. The aa² moiety is preferably of (S)-configuration. Particularly preferred compounds have aa¹ of (R)-configuration and aa² of (S)-configuration. The chiral centre —NH—CH(R¹)—B— is preferably of (R)-configuration. It is considered that commercial formulations will have the chiral centres in (R,S,R) arrangement, as for example in the case of salts of Cbz-Phe-Pro-BoroMpg-OH:

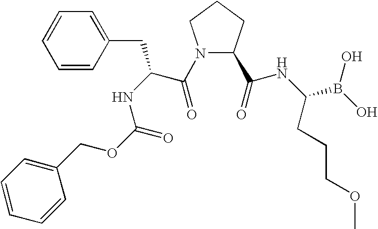

(R,S,R)-TRI 50c Cbz-(R)-Phe-(S)-Pro-(R)-Mpg-B(OH)₂

In preferred embodiments, the various aspects of the disclosure relate to pharmaceutically acceptable base addition salts of the described acids.

The disclosure includes salts of Cbz-(R)-Phe-(S)-Pro-(R)-boroMpg-OH (and of other compounds of the formula X-(R)-Phe-(S)-Pro-(R)-boroMpg-OH) which are at least 90% pure, e.g. at least 95% pure.

The salts are therefore obtainable by contacting a boronic acid drug with a strong base. The disclosure thus contemplates products (compositions of matter) having the characteristics of a reaction product of a boronic acid drug and a strong base. The base is pharmaceutically acceptable.

As suitable salts may be mentioned salts of metals, e.g. of monovalent or divalent metals, and stronger organic bases, for example:

1. Alkali metal salts;

2. Divalent, e.g. alkaline earth metal, salts;

3. Group III metals;

4. Salts of strongly basic organic nitrogen-containing compounds, including:
4A. Salts of guanidines and their analogues;
4B. Salts of strongly basic amine, examples of which include (i) aminosugars and (ii) other amines.

Of the above salts, particularly illustrative are alkali metals, especially Na and Li. Also illustrative are aminosugars.

Specific salts are of the acid boronate though in practice the acid salts may contain a very small proportion of the doubly deprotonated boronate. The term “acid boronate” refers to trigonal —B(OH)₂ groups in which one of the B-OH groups is deprotonated as well as to corresponding tetrahedral groups in equilibrium therewith. Acid boronates have a stoichiometry consistent with single deprotonation.

Accordingly, the disclosure includes base addition salts of the disclosed boronic acids, for example those of Formula (XXX), which have an observed stoichiometry consistent with the organoboronic acid being in the form of a salt of which a single —OH group of the trigonally-represented boronyl group —B(OH)₂ is deprotonated or, in an alternative expression of the same deprotonation state, in which the boronyl group carries a single negative charge and is in a form selected from the group consisting of the following equilibrium species or a combination thereof:

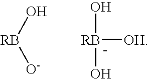

In the above formulae, R represents the organic moiety with which the boron is substituted. For example, in the case of species derived from free acids of formula (XXX), R is the following sub-structure found within formula (XXX):

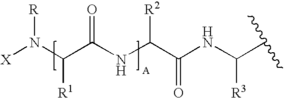

There are to be mentioned pharmaceutically acceptable base addition salts of organoboronic acids of formula (A) below, the salt optionally having an observed stoichiometry consistent with the organoboronic acid being in the form of a salt comprising organoboronate anions and cations and of which a predominant portion has an anion:cation stoichiometry of about n:1, where n is the valency of the cation, formula (A) being: 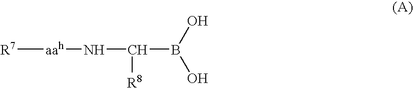
where

R⁷ is X-E′- wherein X is hydrogen or an amino-protecting group and E′ is absent or is a hydrophobic amino acid;

R⁸ is an optionally substituted moiety containing from 1 to 5 carbon atoms selected from the group consisting of alkyl, alkoxy and alkoxyalkyl, the optional substituents being hydroxy or, preferably, halogen (F, Cl, Br, I) and the alkyl moieties being branched or straight chain; and

aa^{h} is a hydrophobic amino acid, or is glycine N-substituted by a C₁-C₁₃ hydrocarbyl group optionally containing in-chain oxygen or sulfur and optionally substituted by a substituent selected from halo, hydroxy and trifluoromethyl.

R⁷ may be X—, or X-Phe or X-Dpa.

R⁸ is preferably not substituted. R⁸ is preferably a C₄ group, e.g. alkyl or alkoxyalkyl, such as 2-methylpropyl or 3-methoxypropyl, for example. In variants of Formula (II), R⁸ is phenyl or benzyl, in either case optionally substituted by —CN or by one or two halogens (e.g. chlorine).

When aa^{h} is N-substituted glycine, the N-substituent is for example a C₃-C₆ hydrocarbyl group comprising a C₃-C₆ hydrocarbyl ring; the hydrocarbyl group may be saturated, for example an exemplary R⁴ group for these compounds is cycloalkyl, e.g. cyclopentyl.

The hydrophobic amino acids may be the same or different and for example be selected from amino acids having a side chain of formula (A) or (B) as defined above, e.g. of formula (C), (D) or (E), and from imino acids as described previously. The disclosure includes a class of salts wherein the organoboronic acid is of formula (II) and the hydrophobic amino acids, being the same or different, have a side chain containing up to 20 carbon atoms and often containing up to 13 carbon atoms or are imino acids. The hydrophobic amino acids may have a side chain as described previously for hydrophobic amino acids contained in the fragment X-E of Formula (I). In a subset of salts containing formula (II) acids, the hydrophobic amino acid is hydrocarbyl or heteroaryl, or which includes both hydrocarbyl and heteroaryl residues. The hydrocarbyl residues optionally contain in-chain oxygen; they may be substituted by, for example, halogen (e.g. 1, 2 or 3 halogen atoms) or hydroxy (but usually not more than one hydroxy group). Alternatively, hydrophobic amino acids may be proline or another imino acid.

In certain variants, R⁷ contains a hydrophobic amino acid which is not Pro or another imino acid. In such embodiments, the hydrophobic amino acid of R⁷ suitably has a side chain of formula (A) or (B) described previously [e.g. of formula (D) or (E)].

aa^{h} may for example be a natural hydrophobic amino acid, e.g. Pro or Phe.

In certain examples X is R⁶—(CH₂)ₚ—C(O)—, R⁶—(CH₂)ₚ—S(O)₂—, R⁶—(CH₂)ₚ—NH—C(O)— or R⁶—(CH₂)ₚ—O—C(O)— wherein p is 0, 1, 2, 3, 4, 5 or 6 (of which 0 and 1 are preferred) and R⁶ is H or a 5 to 13-membered cyclic group optionally substituted by 1, 2 or 3 substituents selected from halogen, amino, nitro, hydroxy, a C₅-C₆ cyclic group, C₁-C₄ alkyl and C₁-C₄ alkyl containing, and/or linked to the 5 to 13-membered cyclic group through, an in-chain O, the aforesaid alkyl groups optionally being substituted by a substituent selected from halogen, amino, nitro, hydroxy and a C₅-C₆ cyclic group. More particularly X is R⁶—(CH₂)ₚ—C(O)— or R⁶—(CH₂)ₚ—O—C(O)— and p is 0 or 1. Said 5 to 13-membered cyclic group is often aromatic or heteroaromatic, for example is a 6-membered aromatic or heteroaromatic group. In many cases, the group is not substituted.

Exemplary X groups are (2-pyrazine) carbonyl, (2-pyrazine) sulfonyl and benzyloxycarbonyl.

The organoboronic acid may be a protease inhibitor, for example a serine protease inhibitor. Thus the disclosure includes salts of a multivalent metal and an organoboronic acid inhibitor of a coagulation serine protease, for example thrombin or Factor Xa. As examples of such organoboronic acids may be mentioned peptide boronates, particularly dipeptides and tripeptides, which in either case may have a protecting group (a non-hydrogen X group) on the N-terminal amino moiety.

In a sub-class of compounds of Formula (A), the symbols R⁷, R⁸ and aa^{h} have the following meanings, the sub-class comprising proteasome inhibitors:

R⁷ is hydrogen or an amino-protecting group, e.g. a previously described amino-protecting group;

R⁸ is C₁-C₅ alkyl; and

aa^{h} is a hydrophobic amino acid, e.g. Phe.

In broad terms, the salts described herein may be considered to correspond to reaction products of an organoboronic acid as described above with a strong base, e.g. a basic metal compound; the salts are however not limited to products resulting from such a reaction and may be obtained by alternative routes. Nonetheless, as previously indicated, the term “base addition salt” is to be understood to refer to a product having the characteristics of a product obtainable by reaction of an organoboronic acid drug with a base, without implying that the product has any particular structure.

The disclosure therefore includes a method for preparing a product, the method comprising contacting an organoboronic acid of formula (XXX) with a pharmaceutically acceptable base. Suitably, the pharmaceutically acceptable base provides cations having a valency n and the base is added in such an amount that the organoboronic acid and the cations are in a stoichiometry of n:1 (organoboronic acid:cations). The method may further comprises formulating the product into an intravenous pharmaceutical formulation. The organoboronic acid may be N-(2-pyrazine) carbonyl-phenylalanine-leucine boronic acid.

The disclosure includes products (compositions of matter) which comprise salts which may be represented by formula (V): 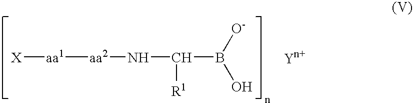
where Yⁿ⁺ is a pharmaceutically acceptable cation obtainable from a strong base, and aa¹, aa², X and R¹ are as defined above. Also included are products in which R¹ is replaced by another R⁹ group. Also included are corresponding compounds in which the peptidoboronyl group of Formula (V) is replaced by another peptidoboronyl group disclosed herein.

One class of salts have a solubility of about 10 mM or more, e.g. of at least about 20 mM, when their solubility is determined as described in the examples at a dissolution of 25mg/ml. More particularly yet they have a solubility of least 50 mM when their solubility is determined as described in the examples at a dissolution of 50 mg/ml.

The disclosure includes salts of boronic acids (I) having an observed stoichiometry consistent with the salt being of (being representable by) the formula “(boronate⁻)ⁿ cationⁿ⁺”. One class of such salts are represented by the formula:
[Cbz-(R)-Phe-(S)-Pro-(R)-Mpg-B(OH)(O⁻)]M⁺
where M⁺ represents a monovalent cation, especially an alkali metal cation. It will be understood that the above representation is a notional representation of a product whose observed stoichiometry is unlikely to be literally and exactly 1:1. In any event, a particular salt is Cbz-(R)-Phe-(S)-Pro-(R)-Mpg-B(OH)₂ monosodium salt (TGN 255). In the above formula, the trigonally-represented boronate represents, as always, boronates which are trigonal, tetrahedral or mixed trigonal/tetrahedral.

Particularly exemplary are products which comprise:

(i) species selected from (a) acids of formula (VIII): X-(R)-Phe-(S)-Pro-(R)-Mpg-B(OH)₂ where X is H or an amino-protecting group, especially Cbz, (b) boronate anions thereof, and

(c) any equilibrium form of the aforegoing (e.g. an anhydride); and

(ii) ions having a valency n in combination with said species, the species and said ions having an observed stoichiometry consistent with a notional species:ion stoichiometry of n:1. In one class of salts, n is 1.

In the following part of this specification, the various possible counter-ions are considered with reference to boronic acids of the following Formula (IIIA): 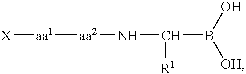
where the various symbols have the meaning ascribed to them previously. Other boronic acid drugs, for example compounds of Formula (XXX) or others referred to in this specification, may of course be used in place of those of Formula (IIIA). Considering the counter-ions in turn, therefore:
1. Monovalent Metal, Especially Alkali Metal Salts

Suitable alkali metals include lithium, sodium and potassium. All of these are remarkably soluble. Lithium and sodium are illustrative because of their high solubility. The lithium and particularly sodium salts are of surprisingly high solubility in relation to potassium amongst others. Sodium is most used in many instances. Salts containing mixtures of alkali metals are contemplated by the disclosure.

The disclosure includes products comprising salts of the formula (VI) 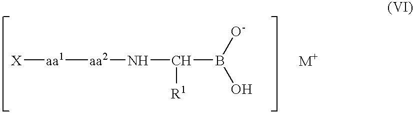
where M⁺ is an alkali metal ion and aa¹, aa², X and R¹ are as defined above, as well as salts in which both hydroxy groups of the boronate group are in salt form (preferably with another identical M⁺ group) and mixtures of such salts. Included also are products wherein R¹ is replaced by another R⁹ group.
2. Divalent, e.g. Alkaline Earth Metal (Group II Metal) Salts

One example of a divalent metal is calcium. Another suitable divalent metal is magnesium. Also contemplated is zinc. The divalent metals are usually used in a boronic acid:metal ratio of substantially 2:1, in order to achieve the preferred monovalent boronate moiety. Salts containing mixtures of divalent metals, e.g. mixtures of alkaline earth metals, are also contemplated.

Further disclosed are products (compositions of matter) which comprise salts which may be represented by the formula (VII): 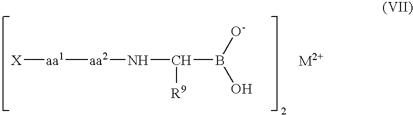
where M²⁺ is a divalent metal cation, e.g. an alkaline earth metal or zinc cation, and aa¹, aa², X and R⁹ are as defined above, as well as salts in which both hydroxy groups of the boronate group are deprotonated and mixtures of such salts. As previously indicated, the boronate may comprise a tetrahedral species.
3. Group III Metals

Suitable Group III metals include aluminium and gallium. Salts containing mixtures of Group III metals are also contemplated.

The disclosure includes products comprising salts of the formula (VIII): 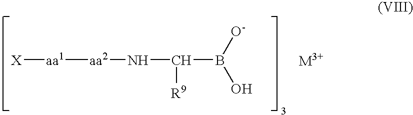
where M³⁺ is a Group III metal ion and aa¹, aa², X and R⁹ are as defined above, as well as salts in which both hydroxy groups of the boronate group are in salt form and mixtures of such salts. As previously indicated, the boronate may comprise a tetrahedral species.
4. Strongly Basic Organic Nitrogen-Containing Compounds

The disclosure includes products obtainable by (having the characteristics of a product obtained by) reaction of a peptide boronic acid as defined above and a strong organic base. Two illustrative classes of organic base are described in sections 4A and 4B below. Particularly preferred are acid salts (in which one of the two boronic —OH groups is deprotonated). Most commonly, the salts contain a single type of organic counter-ion (disregarding trace contaminants) but the disclosure contemplates salts containing mixtures of organic counter-ions; in one sub-class, the different counter-ions all fall within the section 4A family described below or, as the case may be, in the section 2B family below; in another subclass, the salts comprise a mixture of organic counter-ions which are not all from the same family (4A or 4B).

Suitable organic bases include those with a pKb of 7 or more, e.g. 7.5 or more, for example in the region of 8 or more. Bases which are less lipophilic [e.g. have at least one polar functional group (e.g. 1, 2 or 3 such groups) for example hydroxy] are favoured; thus aminosugars are one favoured class of base.

4A. Guanidines and Their Analogues

The guanidino compound (guanidine) may in principle be any soluble and pharmaceutically acceptable compound having a guanidino or a substituted guanidino group, or a substituted or unsubstituted guanidine analogue. Suitable substituents include aryl (e.g. phenyl), alkyl or alkyl interrupted by an ether or thioether linkage and, in any event, typically contain from 1 to 6 and especially 1, 2, 3, or 4 carbon atoms, as in the case of methyl or ethyl. The guanidino group may have 1, 2, 3 or 4 substituent groups but more usually has 1 or 2 substituent groups, for instance on a terminal nitrogen. One class of guanidines is monoalkylated; another class is dialkylated. As guanidine analogues may be mentioned thioguanidines and 2-amino pyridines. Compounds having unsubstituted guanidino groups, for example guanidine and arginine, form one particular class.

Salts containing mixtures of guanidines are contemplated by the disclosure.

A particular guanidino compound is L-arginine or an L-arginine analogue, for example D-arginine, or the D- or, preferably, L-isomers of homoarginine or agmatine [(4-aminobutyl) guanidine]. Less preferred arginine analogues are NG-nitro-L-arginine methyl ester, for example, and constrained guanidine analogues, particularly 2-amino pyrimidines, for example 2,6-quinazolinediamines such as 5,6,7,8-tetrahydro-2,6-quinazolinediamine, for example. The guanidino compound may also be a peptide, for example a dipeptide, containing arginine; one such dipeptide is L-tyrosyl-L-arginine.

Some particular guanidino compounds are compounds of formula (VII): 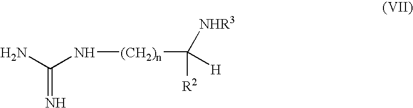
where n is from 1 to 6 and for example at least 2, e.g. 3 or more, and in many instances no more than 5. Most particularly, n is 3, 4 or 5. R² is H or carboxylate or derivatised carboxylate, for example to form an ester (e.g. a C₁-C4 alkyl ester) or amide. R³ is H, C₁-C₄ alkyl or a residue of a natural or unnatural amino acid (e.g. tyrosine). The compounds of formula (IV) are usually of L-configuration. The compounds of formula (IV) are arginine (n=3; R²=carboxyl; R³═H) and arginine derivatives or analogues.

The disclosure includes products comprising salts of the formula (IX) 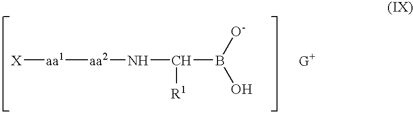
where aa¹, aa², X and R¹ are as defined previously and G⁺ is the protonated form of a pharmaceutically acceptable organic compound comprising a guanidino group or an analogue thereof, as well as salts in which both hydroxy groups of the boronate group are in salt form (preferably with another identical G⁺ group) and mixtures of such salts. Also included are products wherein R¹ is replaced by another R⁹ group.
4B. Strongly Basic Amines

The disclosure includes products obtainable by (having the characteristics of a product obtained by) reaction of a peptide boronic acid as defined above and a strong organic base which is an amine. The amine may in principle be any soluble and pharmaceutically acceptable amine.

It is envisaged that a desirable class of amine includes those having polar functional groups in addition to a single amine group, as such compounds will be more hydrophilic and thus more soluble than others. In certain salts, the or each additional functional group is hydroxy. Some amines have 1, 2, 3, 4, 5 or 6 additional functional groups, especially hydroxy groups. In one illustrative class of amines the ratio of (amino plus hydroxy groups):carbon atoms is from 1:2 to 1:1, the latter ratio being particularly preferred. These amines with one or more additional polar functional groups may be a hydrocarbon, especially an alkane, substituted by the amino group and the additional polar group(s). The amino group may be substituted or unsubstituted and, excluding amino substituents, the polar base may contain, for example, up to 10 carbon atoms; usually there are no less than three such carbon atoms, e.g. 4, 5 or 6. Aminosugars are included in this category of polar bases.

The disclosure includes products comprising salts of the formula (X) 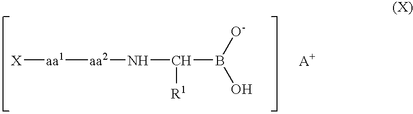
where aa¹, aa², X and R¹ are as defined previously and A⁺ is the protonated form of a pharmaceutically acceptable amine, as well as salts in which both hydroxy groups of the boronate group are in salt form (preferably with another identical A⁺ group) and mixtures of such salts. In one class of such products, A⁺ is the protonated form of an amine described in section 2B(i) below; in another class A⁺ is the protonated form of an amine described in 2B(ii) below. Also included are products in which R¹ is replaced by another R⁹ group.

Two illustrative classes of amine base are described in sections 4B(i) and 4B(ii) below. Particularly preferred are acid salts (in which one of the two boronic —OH groups is deprotonated). Most commonly, the salts contain a single type of amine counter-ion (disregarding trace contaminants) but the disclosure contemplates salts containing mixtures of amine counter-ions; in one sub-class, the different counter-ions all fall within the sub-section 4B(i) family described below or, as the case may be, in the sub-section 4B(ii) family below; in another subclass, the salts comprise a mixture of organic counter-ions which are not all from the same family (4B(i) or 4B(ii)).

4B(i) Aminosugars

The identity of the aminosugar is not critical. Preferred aminosugars include ring-opened sugars, especially glucamines. Cyclic aminosugars are also envisaged as useful. One class of the aminosugars is N-unsubstituted and another, preferred, class is N-substituted by one or two N-substituents (e.g. one). Suitable substituents are hydrocarbyl groups, for example and without limitation containing from 1 to 12 carbon atoms; the substituents may comprise alkyl or aryl moieties or both. Exemplary substituents are C₁, C₂, C₃, C₄, C₅, C₆, C₇ and C₈ alkyl groups, in particular methyl and ethyl, of which methyl is illustrative. Data indicate that aminosugars, especially N-methyl-D-glucamine, are of surprisingly high solubility.

A most preferred aminosugar is N-methyl-D-glucamine: 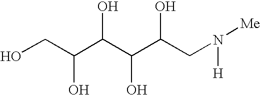
4B(ii) Other Amines

Other suitable amines include amino acids (whether naturally occurring or not) whose side chain is substituted by an amino group, especially lysine.

Some amines are compounds of formula (XI): 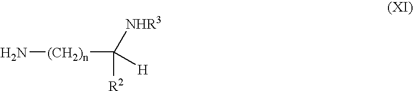
where n, R² and R³ are as defined in relation to formula (IV). The compounds of formula (VI) are usually of L-configuration. The compounds of formula (VI) are lysine (n=4; R²=carboxyl; R³═H) and lysine derivatives or analogues. A most preferred amine is L-lysine.

Other suitable amines are nitrogen-containing heterocycles. At least usually, such heterocyclic compounds are alicyclic; one class of the heterocyclic compounds is N-substituted and another, preferred, class is N-unsubstituted. The heterocycles may contain 6 ring-forming atoms, as in the cases of piperidine, piperazine and morpholine. One class of amines includes N-containing heterocycles substituted by polar substituents, especially hydroxy, e.g. 1, 2 or 3 times.

The disclosure therefore includes amines other than aminosugars which have one or more (e.g. 1, 2, 3, 4, 5 or 6) polar substituents, especially hydroxy, in addition to one amine group. Such compounds may have a ratio of (amino plus hydroxy groups):carbon atoms of 1:2 to 1:1, the latter ratio being particularly preferred.

The disclosure includes mixed salts, i.e. salts containing a mixture of boropeptide moieties and/or counterions but single salts are preferred.

The salts in solid form may contain a solvent, e.g. water. There are included a class of products in which the salts are essentially anhydrous. Also included is a class in which the salts are hydrates.

Synthetic Methods I

1. Peptide/Peptidomimetic Synthesis

The synthesis of boropeptides, including, for example, Cbz-D-Phe-Pro-BoroMpg-OPinacol is familiar to those skilled in the art and described in the prior art mentioned above, including Claeson et al (U.S. Pat. No. 5,574,014 and others) and Kakkar et al (WO 92/07869 and family members including U.S. Pat. No. 5,648,338). It is described also by Elgendy et al Adv. Exp. Med. Biol. (USA) 340:173-178, 1993; Claeson, G. et al Biochem. J. 290:309-312, 1993; Deadman et al J. Enzyme Inhibition 9:29-41, 1995, and by Deadman et al J. Med. Chem. 38:1511-1522, 1995.

The reader is referred also to the following US patents, for example in connection with the synthesis of bortezomib and other boropeptides: U.S. Pat. Nos. 6,617,317; 6,548,668; 6,465,433; 6,297,217; 6,066,730; 5,780,454; and 6,083,903; it will be recalled that all of the aforegoing are incorporated herein by reference.

Stereoselective synthesis with S or R configuration at the chiral B-terminal carbon may be conducted using established methodology (Elgendy et al Tetrahedron. Lett. 33:4209-4212, 1992; WO 92/07869 and family members including U.S. Pat. No. 5648338) using (+) or (−)-pinanediol as the chiral director (Matteson et al J. Am. Chem. Soc. 108:810-819, 1986; Matteson et al Organometallics. 3:1284-1288, 1984). Another approach is to resolve the requisite aminoboronate intermediate (e.g. Mpg-BOPinacol) to selectively obtain the desired (R)-isomer and couple it to the dipeptide moiety (e.g. Cbz-(R)-Phe-(S)-Pro, which is the same as Cbz-D-Phe-L-Pro) which will form the remainder of the molecule.

The boropeptides may be synthesised initially in the form of boronic acid esters, particularly esters with diols. Such diol esters may be converted to the peptide boronic acid as described next.

2. Ester to Acid Conversion

The conversion of boronate esters to the corresponding boronic acid, and of boronic acid to boronate salt, are described next with particular reference to TRI 50c. However, the principles described in this section 2 and following section 3, amongst others, may be applied also to other boronic acids, whether thrombin inhibitors or proteasome inhibitors, e.g. bortezomib, or otherwise. Ester to acid conversion will next be described therefore, with particular but non-limiting reference to TRI 50c.

A peptide boronate ester such as Cbz-(R)-Phe-Pro-BoroMpg-OPinacol may be hydrolysed to form the corresponding acid.

A novel technique for converting a diol ester of a peptide boronic acid of formula (I) into the acid comprises dissolving the diol ester in an ether and particularly a dialkyl ether, reacting the thus-dissolved diol with a diolamine, for example a dialkanolamine, to form a product precipitate, recovering the precipitate, dissolving it in a polar organic solvent and reacting the thus-dissolved product with an aqueous medium, e.g. an aqueous acid, to form the peptide boronic acid. The boronic acid may be recovered from the organic layer of the mixture resulting from the reaction, for example by removing the solvent, e.g. by evaporation under vacuum or distillation. The reaction between the diol ester and the diolamine may be carried out under reflux, for example.

The identity of the diol is not critical. As suitable diols may be mentioned aliphatic and aromatic compounds having hydroxy groups that are substituted on adjacent carbon atoms or on carbon atoms substituted by another carbon. That is to say, suitable diols include compounds having at least two hydroxy groups separated by at least two connecting carbon atoms in a chain or ring. One class of diols comprises hydrocarbons substituted by exactly two hydroxy groups. One such diol is pinacol and another is pinanediol; there may also be mentioned neopentylglycol, 1,2-ethanediol, 1,2-propanediol, 1,3-propanediol, 2,3-butanediol, 1,2-diisopropylethanediol, 5,6-decanediol and 1,2-dicyclohexylethanediol.

The alkyl groups of the dialkyl ether preferably have 1, 2, 3 or 4 carbon atoms and the alkyl groups may be the same or different. An exemplary ether is diethyl ether.

The alkyl groups of the dialkanolamine preferably have 1, 2, 3 or 4 carbon atoms and the alkyl groups may be the same or different. An exemplary dialkanolamine is diethanolamine. The diethanolamine/boronic acid reaction product hydrolyses in water at room temperature and the rate of hydrolysis may be accelerated by adding acid or base.

The polar organic solvent is preferably CHCl₃. Other examples are polyhalogenated alkanes generally and ethyl acetate. In principle, any polar organic solvent is acceptable other than alcohols.

The aqueous acid is suitably a strong inorganic acid at a pH in the region of 1 such as hydrochloric acid, for example.

After reaction with the acid, the reaction mixture is suitably washed with, for example, NH₄Cl or another mild base.

An example of a specific procedure is as follows

1. The pinacol or pinanediol ester of the selected peptide boronic acid is dissolved in diethylether.

2. Diethanolamine is added and the mixture is refluxed at 40° C.

3. The precipitated product is removed (filtered), washed (usually several times) with diethyl ether or another polar organic solvent other than an alcohol, and dried (e.g. by evaporation under vacuum).

4. The dry product is dissolved in a polar organic solvent other than an alcohol, e.g. CHCl₃. Aqueous acid or base is added, e.g. hydrochloric acid (pH 1), and the mixture is stirred for e.g. approximately 1 h at room temperature.

5. The organic layer is removed and washed with NH₄Cl solution.

6. The organic solvent is distilled off and the residual solid product is dried.

The above process results in the formation of what may conveniently be referred to as a “diolamine adduct” of the peptide boronic acids of formula (I), especially such adducts with diethanolamine, and such adducts are themselves included in the disclosure. The molecular structure of such adducts is not known: they might comprise a compound in which the two oxygens and the nitrogen of the diolamine are all coordinated to the boron; they might comprise ions. The adducts are however considered to be esters. A particular novel product included in the disclosure is that obtainable by reacting a pinacol or pinanediol ester of a compound of Formula VIII, particularly (R,S,R)-TRI 50c, and diethanolamine, i.e. the novel product is an (R,S,R)-TRI 50c/diethanolamine “adduct” where the acid is (R,S,R)-TRI 50c.

The diolamine materials of the disclosure may be defined as a composition of matter comprising:
(i) a species of formula (XII) 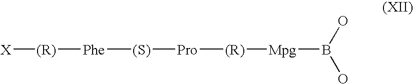
   wherein X is H or an amino protecting group, the boron atom is optionally coordinated additionally with a nitrogen atom, and the valency status of the terminal oxygens is open (they may be attached to a second covalent bond, be ionised as —O⁻, or have some other, for example intermediate, status); and, in bonding association therewith
(ii) a species of formula (XIII) 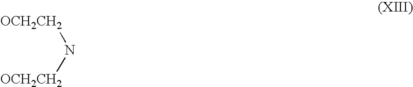
   wherein the valency status of the nitrogen atom and the two oxygen atoms is open. It will be appreciated that the terminal oxygen atoms of the species of formula (IX) and the oxygen atoms of the species of formula (X) may be the same oxygen atoms, in which case the species of formula (X) forms a diol ester with the species of formula (IX).

It will be appreciated that the aforegoing technique comprises an example of a method for recovering an organoboronic acid product, the method comprising providing in a solvent a dissolved mixture comprising the organoboronic acid in a soluble form and a compound having two hydroxy groups and an amino group (i.e. a diolamine), causing or allowing the organoboronic acid and the diolamine to react to form a precipitate, and recovering the precipitate. The soluble form of the organoboronic acid may be a diol ester, as discussed above. The solvent may be an ether, as discussed above. The organoboronic acid may be one of the organoboronic acids referred to in this specification, for example it may be of Formula (I) or (III). The method described in this paragraph is novel and forms an aspect of the disclosure. A recovery method is filtration.

The reaction between the diolamine and the soluble form of the organoboronic acid is suitable carried out at an elevated temperature, for example under reflux.

Another aspect of the disclosure is a method for recovering an organoboron species, comprising
providing, in a form soluble in an ether, an organoboronic acid, for example a drug such as, e.g., a compound of formula (III);
forming a solution of the soluble form in the ether;
combining the solution with a dialkanolamine and allowing or causing the dialkanolamine to react with the soluble form of the organoboronic acid to form an insoluble precipitate; and
recovering the precipitate.

The term “soluble” in the preceding paragraph refers to species which are substantially more soluble in the reaction medium than is the precipitated product. In variants of the method, the ether is replaced by toluene or another aromatic solvent.

The diethanolamine precipitation technique described above is an example of another novel method, which is a method for recovering from ether solution a pinacol or pinanediol ester of a peptide boronic acid, comprising dissolving diethanolamine in the solution, allowing or causing a precipitate to form and recovering the precipitate. The disclosure encompasses variants of this methods in which another diol than pinacol or pinanediol is used.

The precipitated material, i.e. the “adduct”, may be converted into the free organoboronic acid, for example by contacting it with an acid. The acid may be an aqueous acid, for example an aqueous inorganic acid, e.g. as described above. The precipitate may be dissolved, for example in an organic solvent, prior to being contacted with the acid.

The disclosure therefore provides a method for making an organoboronic acid, comprising converting its diolamine reaction product to the acid.

The acid resulting from the methods described in the previous two paragraphs may be converted to a salt of the acid with a multivalent metal, which salt may in turn be formulated into a pharmaceutical composition in parenteral dosage form.

3. Salt Synthesis

In general, the salts may be prepared by contacting the relevant peptide boronic acid with a strong base appropriate to form the desired salt. In the case of metal salts, the metal hydroxides are suitable bases (alternatively, metal carbonates might be used, for example), whilst sometimes it is more convenient to contact the acid with a relevant metal alkoxide (e.g. methoxide), for which purpose the corresponding alkanol is a suitable solvent. Salts with organic bases may be prepared by contacting the peptide boronic acid with the organic base itself. Illustrative salts are acid salts (one —BOH proton replaced) and, to make acid salts with a monovalent cation, the acid and the base are suitably reacted in substantially equimolar quantities. Generally stated, therefore, the usual acid:base molar ratio is substantially n:1, where n is the valency of the cation of the base.

In one procedure, a solution of the peptide boronic acid in a water-miscible organic solvent, for example acetonitrile or an alcohol (e.g. ethanol, methanol, a propanol, for example iso-propanol, or another alkanol), is combined with an aqueous solution of the base. The acid and the base are allowed to react and the salt is recovered. The reaction is typically carried out at ambient temperature (e.g. at a temperature of from 15 to 30° C., e.g. 15 to 25° C.), but an elevated temperature may be used, for example up to the boiling point of the reaction mixture but more usually lower, e.g. a temperature of up to 40° C. or 50° C. The reaction mixture may be allowed to stand or be agitated (usually stirred).

The time during which the acid and the base are allowed to react is not critical but it has been found desirable to maintain the reaction mixture for at least one hour. A period of from one to two hours is usually suitable but longer reaction times may be employed.

The salt may be recovered from the reaction mixture by any suitable method, for example evaporation or precipitation. Precipitation may be carried out by adding an excess of a miscible solvent in which the salt has limited solubility. In one preferred technique, the salt is recovered by evacuating the reaction mixture to dryness. The salt is preferably thereafter purified, for example by redissolving the salt before filtering the resulting solution and drying it, for example by evacuating it to dryness. The redissolution may be performed using water, e.g. distilled water. The salt may then be further purified, for example in order to remove residual water by further redissolution in a suitable solvent, which is advantageously ethyl acetate or THF followed by evaporating to dryness. The purification procedure may be carried out at ambient temperature (say, 15 to 30° C., e.g. 15 to 25° C.), or at a modestly elevated temperature, such as e.g. a temperature not exceeding 40° C. or 50° C.; for example the salt may be dissolved in water and/or solvent by agitating with or without warming to, for example, 37° C.

Also included is a method for drying the salts of the disclosure and other peptide boronic acid salts, comprising dissolving them in an organic solvent, e.g. ethyl acetate or THF, and then evaporating to dryness, e.g. by evacuation.

Generally, preferred solvents for use in purifying the salts are ethyl acetate or THF, or perhaps another organic solvent.

A general procedure for synthesising salts of Cbz-Phe-Pro-BoroMpg-OH is as follows:

Cbz-Phe-Pro-BoroMpg-OH (20.00 g, 38.1 mM) is dissolved in acetonitrile (200ml) with stirring at room temperature. To this solution is added the requisite base in solution in distilled water (190 ml); the base is added as a 0.2M solution for a monovalent cation. The resultant clear solution is allowed to react for example by being left to stand or being agitated, for a usual period, in either case, of from one to two hours. The reaction is typically carried out at ambient temperature (e.g. 15-30° C., e.g. 15 to 25° C.) but alternatively the temperature may be elevated (e.g. up to 30° C., 40° C. or 50° C.). The reaction mixture is then evacuated to dryness under vacuum with its temperature not exceeding 37° C., typically to yield a white brittle solid or an oil/tacky liquid. The oil/tacky liquid is redissolved in the minimum amount of distilled water necessary (200 ml to 4 L), typically with warming (e.g. to 30-40° C.), usually for up to 2 hours. The solution is filtered, suitably through filter paper, and evacuated to dryness, again with the temperature of the solution not exceeding 37° C., or freeze dried. The resultant product is dried under vacuum overnight to normally yield a white brittle solid. If the product is present as an oil or tacky solid then it is dissolved in ethyl acetate and evacuated to dryness to produce the product as a white solid. The white solid is typically a coarse, amorphous powder.

In variations of the aforegoing general procedure, the acetonitrile is replaced by another water-miscible organic solvent, notably an alcohol, as discussed above, especially ethanol, methanol, iso-propanol or another propanol.

Where a boronic acid salt is less soluble in a selected reaction medium for salt formation such that its direct preparation from the corresponding acid and base is inconvenient, the less soluble salt may be prepared from a salt more soluble in the reaction medium.

There is provided also the use of a boronic acid to make a salt of the disclosure. Included also is a method of preparing a product of the disclosure, comprising contacting a boronic acid, e.g. of formula (I), (II) or (III), with a base capable of making such a salt.

The peptide boronic acid of formula (I) used to prepare the pharmaceutical preparations is typically of GLP or GMP quality, or in compliance with GLP (good laboratory practice) or GMP (good manufacturing practice); such acids are included in the disclosure.

Similarly the acids are usually sterile and/or acceptable for pharmaceutical use, and one aspect of the disclosure reside in a composition of matter which is sterile or acceptable for pharmaceutical use, or both, and comprises a peptide boronic acid of formula (I). Such a composition of matter may be in particulate form or in the form of a liquid solution or dispersion.

The intermediate acid may be in isolated form and such isolated acids are included in the disclosure, especially isolated acids which are a peptide boronic acid of formula (VIII):
X—(R)-Phe-(S)-Pro-(R)-Mpg-B(OH)₂ (VIII)
wherein X is H (to form NH₂) or an amino-protecting group.

One typical way of providing the intermediate acids is as a particulate composition consisting predominantly of such a peptide boronic acid, and these compositions are included in the disclosure. The peptide boronic acid often forms at least 75% by weight of the composition and typically at least 85% by weight of the composition, e.g. at least 95% by weight of the composition.

Another typical way of providing the intermediate acids is as a liquid composition consisting of, or consisting essentially of, a peptide boronic acid of formula (II) and a liquid vehicle in which it is dissolved or suspended. The liquid vehicle may be an aqueous medium, e.g. water, or an alcohol, for example methanol, ethanol, isopropanol, or another propanol, another alkanol or a mixture of the aforegoing.

The compositions of the intermediate acids are generally sterile. The compositions may contain the peptide boronic acid in finely divided form, to facilitate further processing.

4. Separation of Stereoisomers

The stereoisomers of a peptide boronic ester or a synthetic intermediate aminoboronate may be resolved in, for example, any known way. In particular, stereoisomers of boronic esters may be resolved by HPLC.

Synthetic Methods II—Stability and Purity of the Compounds

Existing publications teach that organoboronic acids are degraded by oxidation of the C—B bond. See for example Wu et al (see above). Earlier work on the salts of TRI 50c confirmed that these salts and/or intermediates in their preparation are slightly unstable, to the extent that the salts were found to contain a boron-free impurity, designated impurity I, which was evidently generated by C—B bond cleavage. The salts as a class are significantly more stable to such degradation than the free acid.

These earlier TRI 50c salts were made via the general methods described in Examples 5 and 9 of this specification. Impurity I has the following structure:

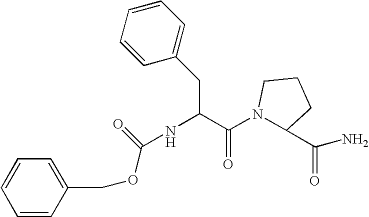

For example, an HPLC chromatogram, prepared using a reverse phase method more particularly described in Example 34, produced the following data for the monosodium salt of TRI 50c, made by following the procedures of Examples 5 and 9 herein:

| | | RT | | | | | % |
|---|---|---|---|---|---|---|---|
| | Name | (min) | Area | Height | Amount | Units | Area |
| 1 | Benzaldehyde | 6.145 | 2487 | 224 | | | 0.39 |
| 2 | Impurity I | 11.022 | 6379 | 539 | | | 1.00 |
| 3 | TRI50c | 11.679 | 628872 | 51108 | 946,063 | ug/ | 98.61 |
| | | | | | | mL | |

Attempts to purify salts contaminated with Impurity I were not successful, and it appeared that, for example, Impurity I was generated from the salts in HPLC columns.

Relative chiral purity of salts made following the general procedure of Examples 5 and 9 was achieved by resolving by HPLC the pinacol ester of TRI 50c, designated TRI 50b, and converting the thus-resolved TRI 50b into the salts. Such an HPLC procedure is not acceptable for normal commercial drug production.

It has further been found that the prior art synthesis summarised earlier under the heading “Aminoboronate Procedure” results, when applied to the synthesis of TRI 50c or an ester thereof, in formation of an impurity designated Impurity IV:

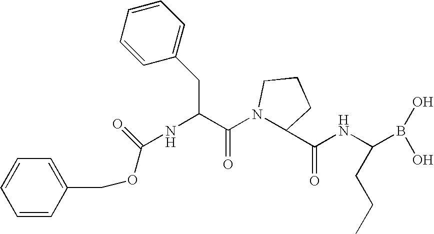

Attempts to separate Impurity IV from TRI 50c have not succeeded. The same applies to TRI 50c salts and esters and the corresponding salts and esters of Impurity IV. No purification technique which has been tried can prevent the presence of Impurity IV if said prior art synthesis is used.

Synthetic Method II—The Methods

Amongst other things, the present disclosure addresses the problems of controlling C-B bond cleavage in organoboronic compounds as well as providing chirally purified salts of TRI 50c and other organoboronic acids on a commercial scale. In this regard, it has been found that C—B bonds seem to be cleaved by a non-oxidative mechanism which occurs in the presence of many solvents, including water and e.g. aqueous acids and bases, amongst others.

It has also been found that chirally-selective precipitation can be used to recover organoboronic acids in high purity.

Thus C—B bond cleavage (and hence in particular generation of Impurity I) may be controlled by:
Selection of acetonitrile as a solvent, where a solvent is required in processing and acetonitrile has the necessary solvation power; in particular acetonitrile is selected in process where a polar solvent is desirable or necessary.
Avoiding excessive contact with water.

In terms of TRI 50c salt production, therefore, the disclosure includes processes comprising one, two or three of the following features:
(i) resolution of the (R,S,S) and (R,S,R) epimers of TRI 50c by chirally selective precipitation using diethanolamine and conveniently, but not necessarily, using as starting material TRI 50c in the form of an ester, for example the pinacol ester;
(ii) control of the duration and/or conditions of hydrolysis of TRI 50c diethanolamine ester, for example as obtained by such precipitation, to control C—B bond breakage;
(iii) use of acetonitrile as solvent for TRI 50c, for example as obtained by such hydrolysis, for the purposes of reacting the TRI 50c with a base to form the salt. Another favourable solvent can be tetrahydrofuran.

As an optional, or even stand-alone, fourth feature, TRI 50c salts may be dried by azeodrying using acetonitrile.

It is considered that C-B bond cleavage may occur by a nucleophilic mechanism, and the disclosure therefore includes methods in which opportunities for nucleophilic attack are minimised.

The above four features, or any one, two or three of them, may be applied to the manufacture and processing of other boronic compounds, particularly acids of formula (I) and their derivatives (e.g. esters and salts).

The disclosure provides in one aspect, therefore, the use of diethanolamine to resolve by selective precipitation the diastereomers of boronic acids of formula (Ia): 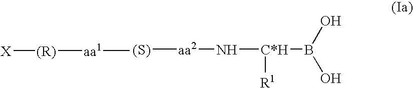
where:
X is H (to form NH₂) or an amino-protecting group;
aa¹ is an amino acid of (R) configuration selected from Phe, Dpa and wholly or partially hydrogenated analogues thereof;
aa² is an imino acid of (S) configuration having from 4 to 6 ring members;
R¹ is a group of the formula —(CH₂)ₛ-Z, where s is 2, 3 or 4 and Z is —OH, —OMe, —OEt or halogen selected from F, Cl, Br or I,
and where C* is a chiral centre.

The starting material may be an acid (Ia) or a derivative thereof capable of forming a diethanolamine ester of the boronic acid. The precipitation selects acids having a chiral centre C* of (R) configuration as precipitate. The precipitate may be recovered and converted to the corresponding boronic acid or a salt thereof. The salt may be made into a pharmaceutical formulation. In practice, the starting material may contain trace amounts of acid in which the fragment aa¹-aa² is not of (R,S) configuration, e.g. it may be at least 99.5% (R,S), and in some cases at least 99.7% (R,S).

For optimised chiral purity and yield, the diethanolamine may be used in an amount of about 1.25±0.1 equivalents based on initial equivalents of boronic acid having a chiral centre C* of (R) configuration.

The initial boronic acid or acid derivative may for example comprise from 50% to 60% molecules having chiral centre C* of (R)-configuration and from 40% to 50% molecules having chiral centre C* of (S)-configuration.

The method opens the way to commercialisation of the boronic acids (Ia) and their derivatives, particularly salts, as pharmaceuticals. Commercial scale products and activities using the boronic acids (Ia) and their derivatives are therefore provided.

In one embodiment, there is provided a process for separating diastereomers of a boronic acid of formula (Ia), comprising:
combining in diethylether solution (A) a boronic species selected from the boronic acid (I) and its esters, the boronic species including molecules having a chiral centre C* of (R) configuration and molecules having a chiral centre C* of (S) configuration, and (B) diethanolamine, the diethanolamine being in an amount of about 1.25±0.1 equivalents based on the boronic species in which the chiral centre C* is of (R) configuration, and mixing to form a mixture;
causing or allowing the boronic species and the diethanolamine to react until a precipitate forms; and
recovering the precipitate.

When the starting material is an ester, it may be an ester of the boronic acid with an alcohol selected from the group consisting of alcohols whose sole potential electron donor heteroatoms are oxygens which, in the boronic ester, correspond to the oxygens of the ester functional group.

In some methods, the diethanolamine is in an amount of from 1.2 to 1.3 equivalents based on the boronic species in which chiral centre C* is of (R) configuration.

There are included processes in which the boronate species is an ester of the boronic acid and a diol, in particular a diol which is not sterically hindered. As exemplary diols may be mentioned pinacol, neopentylglycol, 1,2-ethanediol, 1,2-propanediol, 1,3-propanediol, 2,3-butanediol, 1,2-diisopropylethanediol, or 5,6-decanediol. A particular diol is pinacol.

The boronic species and the diethanolamine may be caused to react by heating the mixture to an elevated temperature, for example the mixture may be refluxed. e.g. for at least 10 hours.

The precipitate may be recovered by filtration. The recovered precipitate may be washed with diethylether. The recovered precipitate, after washing if such takes places, may be dissolved in a solvent selected from CH₂Cl₂ and CHCl₃ and reprecipitated by combining the resulting solution with diethylether. A particular solvent is CH₂Cl₂.

The recovered precipitate (consisting substantially exclusively of an adduct between diethanolamine and the (R,S,R) isomer of the acid) may be converted to the acid of formula (Ia), suitably by hydrolysis, for example by dissolving the precipitate in an organic solvent selected from e.g. halohydrocarbons and combinations thereof, agitating the resulting solution with an aqueous liquid, e.g. an aqueous acid having a pH of below 3, whereby the dissolved precipitate is converted to the formula (Ia) acid, and recovering the formula (Ia) acid by evaporation. The organic solvent may be CH₂Cl₂ or CHCl₃. A particular solvent is CH₂Cl₂. In some processes, organic solvent is further evaporated from the recovered formula (Ia) acid.

The disclosure includes methods in which an ester (particularly a diethanolamine ester) of a organoboronic acid, for example an aminoboronate or peptide boronate such as, e.g. a boronic acid of formula (I) or formula (Ia), is hydrolysed in a manner which controls C—B bond cleavage. In particular, this involves limiting the period of hydrolysis at the selected temperature. In the case of diethanolamine ester hydrolysis, the hydrolysis is suitably carried out at room temperature, or less, for a period not exceeding about 30 minutes, e.g. not exceeding about 20 minutes, and optimally of about 20 minutes. In more general terms, the duration of hydrolysis of the ester is limited to avoid substantial C—B bond breakage, i.e. substantially to avoid generation of the degradation product resulting from such bond breakage. By way of example, the product acid (or a salt produced therefrom) may contain at most about 0.5 % of such degradation product by weight of the total product, e.g. less than about 0.3 wt % and often less than about 0.2 wt %. The content of C—B bond degradation product may be about 0.1 wt % or less. In particular instances, there is no more than about 0.05% degradation product as determined by reverse phase HPLC (see Example 43 below). Included are boronic acids and their base addition salts in which there is no C—B degradation product detectable by the HPLC technique of Example 43, or about such amount; of course, hydrolysis methods which result in boronic acids having such a level of purity are also included. In the case of TRI 50c and its salts, the degradation product of C—B bond cleavage of which it is substantially free is Impurity I; base addition salts of TRI 50c have been prepared in which Impurity I was not detected with the initial HPLC analysis.

The disclosure includes methods in which an ester of a boronic acid (I) or formula (Ia), particularly a diethanolamine ester, is hydrolysed in a manner which controls C—B bond cleavage. In particular, this involves limiting the period of hydrolysis at the selected temperature. In the case of diethanolamine ester hydrolysis, the hydrolysis is suitably carried out at room temperature, or less, for a period not exceeding about 30 minutes, e.g. not exceeding about 20 minutes, and optimally of about 20 minutes.

Thus the recovered precipitate referred to in the last paragraph but one may be hydrolysed using an aqueous acid, particularly 2% hydrochloric acid or another mineral acid of similar pH, for no more than about 30 minutes at about room temperature, or less. Suitably, the precipitate is dissolved in a, non-nucleophilic organic solvent (e.g. a halohydrocarbon or halohydrocarbon mixture for example CH₂Cl₂) and the resulting solution is contacted with the aqueous acid for a period as previously described. The precipitate is thereby hydrolysed to form the free acid of formula (I) or (Ia), which remains in the organic solvent. The organic solvent may be separated from the aqueous medium and then evaporated to obtain solid acid of formula (I) or (Ia).

There are included processes in which a formula (I) or formula (Ia) acid, for example obtained as described in the preceding paragraph, is dried. In a class of processes, the formula (I) acid is dried when it is in the organic solvent by contacting the solvent with a hygroscopic solid.

Included are processes in which the formula (I) or formula (Ia) acid, when in the organic solvent, is washed with an aqueous ammonium salt.

Chirally purified boronic acid may be converted to a pharmaceutically acceptable base addition salt thereof, in particular by dissolving the acid in acetonitrile, combining the resultant solution with an aqueous solution or suspension of a pharmaceutically acceptable base, and causing or allowing the base and the acid to react, then evaporating to dryness to obtain an evaporation residue. The step of causing or allowing the acid and the base to react may comprise agitating the combination of the acetonitrile solution of the acid and the aqueous solution or suspension of the base at a temperature of not more than 35° C. and often of not more than 30° C., e.g. not more than 25° C.; an optimal temperature is room temperature, in which case a reaction time of about 2 hours might be appropriate. The process may further comprise:
(i) redissolving the evaporation residue in acetonitrile and evaporating the resulting solution to dryness; and
(ii) repeating step (i) as often as necessary to obtain a dry evaporation residue.

In some processes the dry evaporation residue is dissolved in acetonitrile or tetrahydrofuran to form a solution, and the solution is combined with (e.g. slowly added to, at a rate sufficiently slow to avoid lump formation) a 3:1 to 1:3 v/v mixture of diethylether and an aliphatic or cycloaliphatic solvent to form a precipitate, said solution being added to the diethylether/(cyclo)aliphatic solvent mixture in a ratio (solution:mixture) of from 1:5 to 1:15 v/v. The precipitate is recovered and some or substantially all remaining solvent is removed from the recovered precipitate whilst maintaining the temperature at no more than 35° C., e.g. is removed under reduced pressure. Included are processes in which the temperature at the start of the drying process is about 10° C. and is increased during the process to 35° C. The aliphatic or cycloaliphatic solvent may have 6, 7 or 8 carbon atoms; the solvent may be an alkane, for example an n-alkane, e.g. n-heptane. Some reactions may be carried out at ambient temperature, which may e.g. be 15-30° C., e.g. 20-30° C.; sometimes ambient temperature may be room temperature.

The salts produced by the invention may contain a trace amount of the aliphatic or cycloaliphatic solvent, e.g. an amount of less than 0.1%, particularly less than 0.01%, for example an amount of about 0.005%.

In the process for making the salt, the base may comprise a cation of valency n and be used in a stoichiometry (boronic acid:base) of about n:1. In particular processes, the base is an alkali metal or alkaline earth metal base, for example an alkali metal hydroxide or an alkaline earth metal hydroxide. As one base may be mentioned sodium hydroxide. As another base may be mentioned calcium hydroxide. The disclosure includes processes in which the base is sodium hydroxide and the dry evaporation residue is dissolved in acetonitrile. The disclosure includes processes in which the base is calcium hydroxide and the dry evaporation residue is dissolved in tetrahydrofuran.

The disclosure is not limited as to the method by which the boronic acids of Formula (I) or Formula (Ia) are obtained (for example as an ester thereof). However, in one class of subject matter, the Formula (I) acid has an R¹ group of the formula —(CH₂)S—O—R³ in which R³ is methyl or ethyl and s is independently 2, 3 or 4, and the Formula (I) acid is prepared via an intermediate of Formula (XXV):
(HO)₂B—(CH₂)ₛ—O—R³ (XXV),
which intermediate is made by reaction between a borate ester and a suitable 1-metalloalkoxyalkane.

A novel aspect of the disclosure comprises the Formula (XXV) intermediates.

The Formula (XXV) intermediates may be made by reacting a 1-metalloalkoxyalkane, where the alkoxyalkane is of the formula —(CH₂)ₛ—O—R³, with a borate ester to form a compound of Formula (XXV).

It will be appreciated that the above method provides a general procedure for making alkoxyalkylboronic acids, which may be presented by the formula R^{Z}—O—R^{Y}—B(OH)₂. Such alkoxyalkylboronic acids may be converted to aminoboronates, and the aminoboronates may be derivatised at their amino group to form an amide bond linked to another moiety. In other words, the aminoboronates may be converted to boropeptides. The method will now be described further with non-limiting reference to compounds of Formula (XXV).

The starting materials for the reaction may be a metalloalkoxyalkane, e.g. a Grignard reagent, obtainable from 1-haloalkoxyalkane of the formula Hal-(CH₂)ₛ—O—R³ (where Hal is a halogen) and a borate ester. The metal is in particular magnesium. Another metal is lithium, in which case the metallo reagent may be prepared by reacting the 1-haloalkoxyalkane with butyl lithium. Where the method includes preparation of the metallo reagent from the haloalkoxyalkane, the haloalkoxyalkane may be a chloroalkoxyalkane; the corresponding bromo compounds may also be used. To make a Grignard reagent, magnesium may be reacted with the haloalkoxyalkane.

Suitable borate esters are esters of mono- and di-functional alcohols (e.g. of EtOH, MeOH, BuOH, pinacol, glycol, pinanediol etc). For example, the ester may be of the formula B(OR^{a})(OR^{b})(OR^{c}) where R^{a}, R^{b} and R^{c} and C₁-C₄ alkyl and may be the same as each other.

An exemplary procedure for making a Formula (XOV) intermediate, illustrated with reference to methoxypropane as the alkoxyalkane species, is:

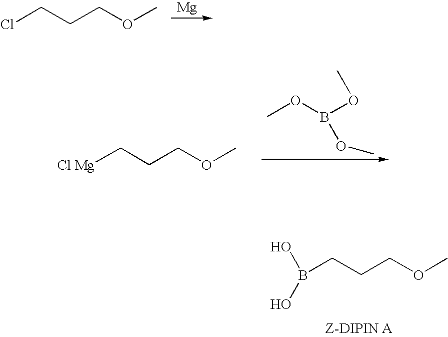

The reactions are suitably carried out in an organic solvent, e.g. THF.

The above-described procedure for making alkoxyalkylboronic acids avoids generation of Impurity IV (see above), or its analogues in those cases where the end product is not TRI 50c or a derivative (salt, ester etc) thereof. The procedure therefore provides a unique route to making TRI 50c, its esters and salts, uncontaminated by Impurity IV, and for making other aminoboronic acids which are substituted α- to the boron by an alkoxyalkyl group and are uncontaminated by impurities analogous to Impurity IV.

An alkoxyalkylboronic acid, i.e. a compound which may be represented by the formula R^{Z}—O—R^{Y}—B(OH)₂, may be converted to an aminoboronic compound, for example a boropeptide, by any suitable procedure, e.g. one known in the art. A reaction scheme for making alkoxyalkylboronic acids into aminoboronates, and for converting aminoboronates into peptide boronates is illustrated with reference to synthesis of TRI 50c at the start of the Examples of this specification. The reaction scheme may be modified as desired, e.g.: diethanolamine precipitation and subsequent steps may be omitted, and/or reagent substitutions may be made. For example, pinacol may be replaced by another diol. LDA is a non-nucleophilic strong base and may be replaced by another such base. Other examples include, but are not limited to, lithium diisopropylamide, lithium 2,2,6,6-tetramethylpiperidine, 1-lithium 4-methylpiperazide, 1,4-dilithium piperazide, lithium bis(trimethylsilyl) amide, sodium bis(trimethylsilyl)amide, potassium bis(trimethylsilyl)amide, isopropyl magnesium chloride, phenyl magnesium chloride, lithium diethylamide, and potassium tert-butoxide. The reactions may be carried out in any suitable solvent: where n-heptane is used in the Examples, it may be replaced by another inert non-polar solvent, e.g. another aliphatic or cycloaliphatic solvent, for example an alkane, e.g. an n-alkane.

Thus, the disclosure includes a process for making an aminoboronate of Formula (XXI) 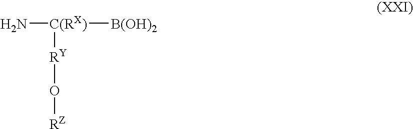
wherein

R^{X} is H or a substituent which does not prevent synthesis;

R^{Y} is alkylene; and

R^{Z} is alkyl,

the process comprising reacting a 1-metalloalkoxyalkane with a borate ester to form a boronic acid of the formula R^{Z}—O—R^{Y}—B(OH)₂, esterifying the acid, contacting the esterified acid with CH₂Cl₂ and ZnCl₂ in the presence of a strong base, contacting the resultant produce with LiHMDS and in turn contacting the resultant product with hydrogen chloride.

The product is free of contaminant of Formula (XXII):
H₂N—C(R^{X})(R^{Y})—B(OH)₂ (XXII).

The aminoboronate (XXI) may be reacted with an amino acid or peptide (which in either case may be suitably protected) to form a peptide boronate. In general terms, therefore, the disclosure includes peptidoboronic acids of Formula (XXIII): 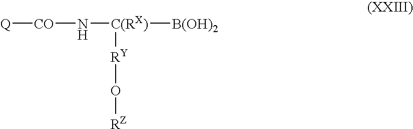
Q-CO comprises at least an amino acid residue;

R^{X} is H or a substituent which does not prevent synthesis;

R^{Y} is alkylene;

R^{Z} is alkyl,

which organoboronic acid is free of an impurity of Formula (XMIV):

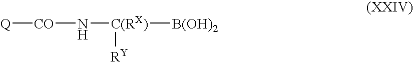

The disclosure further includes derivatives of Formula (XXIII) acids (e.g. acid or base addition salts, esters) which are free of Formula (XXIV) impurity and derivatives thereof.

The exact identity of R^{Y} and R^{Z} is dependent on the identity of the end product, and not part of the process or its benefits.

It will be appreciated from the aforegoing that the above described methods may be used in the manufacture of organoboronic acids salts as described. It is not necessary for sequential steps to be carried out as one operation or at the same site: they may be performed in this way or different processes (different parts of the overall synthesis) may be distributed in time and/or space. Particular end product salts are monosodium, monolithium, hemicalcium and hemimagnesium salts, for example of TRI 50c.

Generally, the reactions may suitably be carried out with a non-nucleophilic solvent. Where a nucleophilic solvent is present, minimum contact is preferred, for example in the case of hydrolysis of diethanolamine esters.

The High Purity Products

The “high purity products” of the invention include inter alia boronic acids, diethanolamine esters and salts obtainable by (having the characteristics of a product obtained by) the disclosed methods. Also included are products obtained directly or indirectly by the disclosed methods.

Particular products of the disclosure are base addition salts of a boronic acid of formula (I) having the chiral purity of such salt when prepared by a method described herein, as well as such boronic acids having a chiral purity obtainable by a method described herein.

Included are esters of boronic acids of formula I (for example, diethanolamine esters), the free acids of formula (I) and salts of the free acid which comprise the (R,S,R) diastereomer in a diastereomeric excess over the (R,S,S) diastereomer of about 95% or more. The (R,S,R) isomer may be in a diastereomeric excess of at least about 98%, and optionally of about 99% or more, e.g. about 99.5% or more. Further included are salts having a diastereomeric excess [(R,S,R) over (R,S,S)] of about 99.5% or more and purity as measured by % HPLC peak area of at least 95% when determined by the method of Example 5; in particular, the salt is a metal salt of TRI 50c, e.g. an alkali metal or alkaline earth metal salt.

Other products are base addition salts of a boronic acid of formula (I) having the purity of such salt when prepared by a method described herein.

Product identities will be apparent from the preceding description and the following examples. In addition, products of the disclosure are described in the claims. Of particular note are the data in Example 43, indicating that the processes of the invention can remarkably achieve end product salts free of impurities detectable by the described HPLC method. In other instances, the salts are substantially free of impurities, e.g. at least 98% pure, more usually at least 99% pure, e.g. at least 99.5% pure, in terms of reverse phase (RP) HPLC percentage peak area. Salts may at least 99.3%, 99.4%, 99.5% 99.6%, 99.7%, 99.8% or 99.9% pure, in terms of reverse phase (RP) HPLC percentage peak area. Suitable RP HPLC procedures comply with reference 1 and/or reference 2 and/or reference 3 of Example 43. Included also are products at least substantially free of Impurity I and analogues, products free of Impurity IV and analogues, and products containing small traces of non-polar solvent, e.g. n-heptane. The trace amount of non-polar solvent may be less than 0.2%, 0.1%, 0.05%, 0.01% or 0.005% as determined by GC-headspace chromatography.

Also to be mentioned is Cbz-(R)-Phe-(S)-Pro-(R)-Mpg-B(OH)₂, and the salts thereof, substantially free of Impurity I.

A further class of compounds comprises Cbz-(R)-Phe-(S)-Pro-(R)-Mpg-B(OH)₂, and the esters and salts thereof, substantially free of Impurity IV

Included also are salts containing less than 410 ppm acetonitrile.

Some salts contain impurities of less than 10,000 ppm, 5000 ppm, 1000 ppm, or 500 ppm.

Use of Products of the Disclosure

1. Thrombin Inhibitors

The thrombin inhibitory products of the disclosure are anti-thrombogenic. They are therefore useful for inhibiting thrombin. There are therefore provided compounds which have potential for controlling haemostasis and especially for inhibiting coagulation, for example in the treatment or prevention of secondary events after myocardial infarction. The medical use of the compounds may be prophylactic (including to treat thrombosis as well as to prevent occurrence of thrombosis) as well as therapeutic (including to prevent re-occurrence of thrombosis or secondary thrombotic events).

The salts may be employed when an anti-thrombogenic agent is needed. Further, it has been found that the salts, including those of boronic acids of Formula (III), are beneficial in that the class is useful for treating arterial thrombosis by therapy or prophylaxis. The disclosed salts are thus indicated in the treatment or prophylaxis of thrombosis and hypercoagulability in blood and tissues of animals including man. The term “thrombosis” includes inter alia atrophic thrombosis, arterial thrombosis, cardiac thrombosis, coronary thrombosis, creeping thrombosis, infective thrombosis, mesenteric thrombosis, placental thrombosis, propagating thrombosis, traumatic thrombosis and venous thrombosis.

It is known that hypercoagulability may lead to thromboembolic diseases.

Examples of venous thromboembolism which may be treated or prevented with compounds of the disclosure include obstruction of a vein, obstruction of a lung artery (pulmonary embolism), deep vein thrombosis, thrombosis associated with cancer and cancer chemotherapy, thrombosis inherited with thrombophilic diseases such as Protein C deficiency, Protein S deficiency, antithrombin III deficiency, and Factor V Leiden, and thrombosis resulting from acquired thrombophilic disorders such as systemic lupus erythematosus (inflammatory connective tissue disease). Also with regard to venous thromboembolism, compounds of the disclosure are useful for maintaining patency of indwelling catheters.

Examples of cardiogenic thromboembolism which may be treated or prevented with compounds of the disclosure include thromboembolic stroke (detached thrombus causing neurological affliction related to impaired cerebral blood supply), cardiogenic thromboembolism associated with atrial fibrillation (rapid, irregular twitching of upper heart chamber muscular fibrils), cardiogenic thromboembolism associated with prosthetic heart valves such as mechanical heart valves, and cardiogenic thromboembolism associated with heart disease.

Examples of conditions involving arterial thrombosis include unstable angina (severe constrictive pain in chest of coronary origin), myocardial infarction (heart muscle cell death resulting from insufficient blood supply), ischemic heart disease (local ischemia due to obstruction (such as by arterial narrowing) of blood supply), reocclusion during or after percutaneous transluminal coronary angioplasty, restenosis after percutaneous transluminal coronary angioplasty, occlusion of coronary artery bypass grafts, and occlusive cerebrovascular disease. Also with regard to arterio-venous (mixed) thrombosis, anti-thrombotic compounds of the disclosure are useful for maintaining patency in arteriovenous shunts.

Other conditions associated with hypercoagulability and thromboembolic diseases which may be mentioned inherited or acquired deficiencies in heparin cofactor II, circulating antiphospholipid antibodies (Lupus anticoagulant), homocysteinemia, heparin induced thrombocytopenia and defects in fibrinolysis.

Particular uses which may be mentioned include the therapeutic and/or prophylactic treatment of venous thrombosis and pulmonary embolism. Preferred indications envisaged for the products of the disclosure (notably the salts of TRI 50c) include:
Prevention of venous thromboembolic events (e.g. deep vein thrombosis and/or pulmonary embolism). Examples include patients undergoing orthopaedic surgery such as total hip replacement, total knee replacement, major hip or knee surgery; patients undergoing general surgery at high risk for thrombosis, such as abdominal or pelvic surgery for cancer; and in patients bedridden for more than 3 days and with acute cardiac failure, acute respiratory failure, infection.
Prevention of thrombosis in the haemodialysis circuit in patients, in patients with end stage renal disease.
Prevention of cardiovascular events (death, myocardial infarction, etc) in patients with end stage renal disease, whether or not requiring haemodialysis sessions.
Prevention of venous thrombo-embolic events in patients receiving chemotherapy through an indwelling catheter.
Prevention of thromboembolic events in patients undergoing lower limb arterial reconstructive procedures (bypass, endarteriectomy, transluminal angioplasty, etc).
Treatment of venous thromboembolic events.
Prevention of cardiovascular events in acute coronary syndromes (e.g. unstable angina, non Q wave myocardial ischaemia/infarction), in combination with another cardiovascular agent, for example aspirin (acetylsalicylic acid; aspirin is a registered trade mark in Germany), thrombolytics (see below for examples), antiplatelet agents (see below for examples).
Treatment of patients with acute myocardial infarction in combination with acetylsalicylic acid, thrombolytics (see below for examples).

The thrombin inhibitors of the disclosure are thus indicated both in the therapeutic and/or prophylactic treatment of all the aforesaid disorders.

In one method, the products of the disclosure are used for the treatment of patients by haemodialysis, by providing the product in the dialysis solution, as described in relation to other thrombin inhibitors in WO 00/41715. The disclosure therefore includes dialysing solutions and dialysing concentrates which comprise a product of the disclosure, as well as a method of treatment by dialysis of a patient in need of such treatment, which method comprises the use of a dialysing solution including a low molecular weight thrombin inhibitor. Also included is the use of an anti-thrombotic product of the disclosure for the manufacture of a medicament for the treatment by dialysis of a patient, in which the anti-thrombotic product of the disclosure is provided in the dialysing solution.

In another method, the products of the disclosure are used to combat undesirable cell proliferation, as described in relation to other thrombin inhibitors in WO 01/41796. The undesirable cell proliferation is typically undesirable hyperplastic cell proliferation, for example proliferation of smooth muscle cells, especially vascular smooth muscle cells. The products of the disclosure particularly find application in the treatment of intimal hyperplasia, one component of which is proliferation of smooth muscle cells. Restenosis can be considered to be due to neointimal hyperplasia; accordingly intimal hyperplasia in the context of the disclosure includes restenosis.

The products of the disclosure are also contemplated for the treatment of ischemic disorders. More particularly, they may be used in the treatment (whether therapeutic or prophylactic) of an ischemic disorder in a patient having, or at risk of, non-valvular atrial fibrillation (NVAF) as described in relation to other thrombin inhibitors in WO 02/36157. Ischemic disorders are conditions whose results include a restriction in blood flow to a part of the body. The term will be understood to include thrombosis and hypercoagulability in blood, tissues and/or organs. Particular uses that may be mentioned include the prevention and/or treatment of ischemic heart disease, myocardial infarction, systemic embolic events in e.g. the kidneys or spleen, and more particularly of cerebral ischemia, including cerebral thrombosis, cerebral embolism and/or cerebral ischemia associated with non-cerebral thrombosis or embolism (in other words the treatment (whether therapeutic or prophylactic) of thrombotic or ischemic stroke and of transient ischemic attack), particularly in patients with, or at risk of, NVAF.

The products of the disclosure are also contemplated for the treatment of rheumatic/arthritic disorders, as described in relation to other thrombin inhibitors in WO 03/007984. Thus, the products of the disclosure may be used in the treatment of chronic arthritis, rheumatoid arthritis, osteoarthritis or ankylosing spondylitis

Moreover, the products of the disclosure are expected to have utility in prophylaxis of re-occlusion (i.e. thrombosis) after thrombolysis, percutaneous trans-luminal angioplasty (PTA) and coronary bypass operations; the prevention of re-thrombosis after microsurgery and vascular surgery in general. Further indications include the therapeutic and/or prophylactic treatment of disseminated intravascular coagulation caused by bacteria, multiple trauma, intoxication or any other mechanism; anticoagulant treatment when blood is in contact with foreign surfaces in the body such as vascular grafts, vascular stents, vascular catheters, mechanical and biological prosthetic valves or any other medical device; and anticoagulant treatment when blood is in contact with medical devices outside the body such as during cardiovascular surgery using a heart-lung machine or in haemodialysis.

The products of the disclosure are further indicated in the treatment of conditions where there is an undesirable excess of thrombin without signs of hypercoagulability, for example in neurodegenerative diseases such as Alzheimer's disease. In addition to its effects on the coagulation process, thrombin is known to activate a large number of cells (such as neutrophils, fibroblasts, endothelial cells and smooth muscle cells). Therefore, the compounds of the disclosure may also be useful for the therapeutic and/or prophylactic treatment of idiopathic and adult respiratory distress syndrome, pulmonary fibrosis following treatment with radiation or chemotherapy, septic shock, septicaemia, inflammatory responses, which include, but are not limited to, edema, acute or chronic atherosclerosis such as coronary arterial disease, cerebral arterial disease, peripheral arterial disease, reperfusion damage, and restenosis after percutaneous trans-luminal angioplasty (PTA).

The salts may also be useful in the treatment of pancreatitis.

The salts described herein are further considered to be useful for inhibiting platelet procoagulant activity. The disclosure provides a method for inhibiting platelet pro-coagulant activity by administering a salt of a boronic acid described herein to a mammal at risk of, or suffering from, arterial thrombosis, particularly a human patient. Also provided is the use of such salts for the manufacture of medicaments for inhibiting platelet procoagulant activity.

The use of products of the disclosure as inhibitors of platelet pro-coagulant activity is predicated on the observation that the boronic acids described herein are indicated to be effective at inhibiting arterial thrombosis as well as venous thrombosis.

Indications involving arterial thrombosis include acute coronary syndromes (especially myocardial infarction and unstable angina), cerebrovascular thrombosis and peripheral arterial occlusion and arterial thrombosis occurring as a result of atrial fibrillation, valvular heart disease, arterio-venous shunts, indwelling catheters or coronary stents. Accordingly, in another aspect there is provided a method of treating a disease or condition selected from this group of indications, comprising administering to a mammal, especially a human patient, a salt of the disclosure. The disclosure includes products for use in an arterial environment, e.g. a coronary stent or other arterial implant, having a coating which comprises a salt according to the disclosure.

The salts of the disclosure may be used prophylactically to treat an individual believed to be at risk of suffering from arterial thrombosis or a condition or disease involving arterial thrombosis or therapeutically (including to prevent re-occurrence of thrombosis or secondary thrombotic events).

There is therefore included the use of selective thrombin inhibitors (organoboronic acid salts) described herein for treatment of the above disorders by prophylaxis or therapy as well as their use in pharmaceutical formulations and the manufacture of pharmaceutical formulations.

2. Proteasome Inhibitors

The proteasome inhibitory products of the disclosure may be used for the purposes described in the following U.S. Pat. Nos. 6,617,317; 6,548,668; 6,465,433; 6,297,217; 6,066,730; 5,780,454; and 6,083,903; all of the aforegoing are incorporated herein by reference.

Thus, the proteasome inhibitors are useful for treating such conditions as tissue rejection, arthritis, local infections, dermatoses, inflammatory bowel diseases, autoimmune diseases, etc. The proteasome inhibitors of the present invention can be employed to prevent the rejection or inflammation of transplanted tissue or organs of any type, for example, heart, lung, kidney, liver, skin grafts, and tissue grafts.

Compounds of the present invention inhibit the growth of cancer cells. Thus, the compounds can be employed to treat cancer, psoriasis, restenosis or other cell proliferative diseases in a patient in need thereof.

By the term “treatment of cancer” or “treating cancer” is intended description of an activity of compounds of the present invention wherein said activity prevents or alleviates or ameliorates any of the specific phenomena known in the art to be associated with the pathology commonly known as “cancer.” The term “cancer” refers to the spectrum of pathological symptoms associated with the initiation or progression, as well as metastasis, of malignant tumors. By the term “tumor” is intended, for the purpose of the present invention, a new growth of tissue in which the multiplication of cells is uncontrolled and progressive. The tumor that is particularly relevant to the invention is the malignant tumor, one in which the primary tumor has the properties of invasion or metastasis or which shows a greater degree of anaplasia than do benign tumors.

Thus, “treatment of cancer” or “treating cancer” refers to an activity that prevents, alleviates or ameliorates any of the primary phenomena (initiation, progression, metastasis) or secondary symptoms associated with the disease. Cancers that are treatable are broadly divided into the categories of carcinoma, lymphoma and sarcoma. Examples of carcinomas that can be treated by the composition of the, present invention include, but are not limited to: adenocarcinoma, acinic cell adenocarcinoma, adrenal cortical carcinomas, alveoli cell carcinoma, anaplastic carcinoma, basaloid carcinoma, basal cell carcinoma, bronchiolar carcinoma, bronchogenic carcinoma, renaladinol carcinoma, embryonal carcinoma, anometroid carcinoma, fibrolamolar liver cell carcinoma, follicular carcinomas, giant cell carcinomas, hepatocellular carcinoma, intraepidermal carcinoma, intraepithelial carcinoma, leptomanigio carcinoma, medullary carcinoma, melanotic carcinoma, menigual carcinoma, mesometonephric carcinoma, oat cell carcinoma, squamal cell carcinoma, sweat gland carcinoma, transitional cell carcinoma, and tubular cell carcinoma. Sarcomas that can be treated by the composition of the present invention include, but are not limited to: amelioblastic sarcoma, angiolithic sarcoma, botryoid sarcoma, endometrial stroma sarcoma, ewing sarcoma, fascicular sarcoma, giant cell sarcoma, granulositic sarcoma, immunoblastic sarcoma, juxaccordial osteogenic sarcoma, coppices sarcoma, leukocytic sarcoma (leukemia), lymphatic sarcoma (lympho sarcoma), medullary sarcoma, myeloid sarcoma (granulocitic sarcoma), austiogenci sarcoma, periosteal sarcoma, reticulum cell sarcoma (histiocytic lymphoma), round cell sarcoma, spindle cell sarcoma, synovial sarcoma, and telangiectatic audiogenic sarcoma. Lymphomas that can be treated by the composition of the present invention include, but are not limited to: Hodgkin's disease and lymphocytic lymphomas, such as Burkitt's lymphoma, NPDL, NML, NH and diffuse lymphomas.

The compounds may be used for the treatment of multiple myeloma.

Amounts and regimens for the administration of proteasome inhibitors and compositions of the invention can be determined readily by those with ordinary skill in the clinical art of treating cancer-related disorders such as the primary phenomena (initiation, progression metastasis) or secondary symptoms associated with the disease. Generally, the dosage of the composition of the invention will vary depending upon considerations such as: type of composition employed; age; health; medical conditions being treated; kind of concurrent treatment, if any, frequency of treatment and the nature of the effect desired; extent of tissue damage; gender; duration of the symptoms; and, counter indications, if any, and other variables to be adjusted by the individual physician. A desired dosage can be administered in one or more applications to obtain the desired results. Pharmaceutical compositions containing the proteasome inhibitors of the invention can be provided in unit dosage forms.

Administration and Pharmaceutical Formulations

1. Aqueous Solutions

It may be desirable to make aqueous solutions of boronic acid drugs for administering them. It has been found possible to form surprisingly concentrated boronate salt solutions (of up to about 600mg/ml in the case of TRI 50c monosodium salt) at a pH of about 9.5. However, a solution with a pH of 9.5 may be unacceptable or undesirable. Accordingly, a pharmaceutically acceptable organic acid may be included in the particulate formulation in an amount selected to reduce the pH to a value at which the solution is more acceptable but at which a solution of drinkable quantity (e.g. about 50 ml to about 150 ml) may be formed by reconstituting the particulate formulation. As the organic acid may be mentioned citric acid, tartaric acid or malic acid, for example. In many instances, citric acid is chosen.

Experiments have been performed to test the solubility of TRI 50c monosodium salt at different pH values. All the experiments were conducted using a quantity of the salt equivalent to 600 mg TRI 50c free acid. In a first series of experiments, this amount of the salt was dissolved in 50 ml water to form a solution of approximately pH 9.5. Dilute aqueous HCl was added to determine how much the pH could be reduced before precipitation occurred. It was found that the salt tended to precipitate when the pH of the reconstituted solution was reduced below 9 and the pH of a reconstituted liquid having this concentration of salt may therefore be maintained at 9 or more, e.g. 9.2 or more, to keep the salt in solution.

In a second series of experiments, the same amount of the salt was dissolved in 150 ml water, and citric acid was added. It was found that the pH could be reduced to a value of 3.7-3.8 using citric acid before precipitation occurred. In other words if, in the case of a salt dosage equivalent to 600 mg TRI 50c, the patient instructions are to prepare a solution in at least 150 ml water, a quantity of organic acid (e.g. citric acid) can be included in the formulation which will reduce the pH to a value of, say, not less than 4, without a risk of precipitation. Since acid solutions tend to be more palatable than alkaline ones, and citric acid is a common flavouring agent, this behaviour of the salt is highly beneficial. In practical terms, up to 200 mg citric acid may be combined with TRI 50c monosodium salt (600 mg, calculated as TRI 50c) for a preparation to be reconstituted in 150 ml water or more. In general, it is contemplated that the boronate will be formulated to form a reconstituted solution having a pH of from 4 to 8, e.g. 4 to 7, optionally 5 to 6.

Of course, the absolute amount of citric or other acid would be varied with (i) the absolute amount of the salt and (ii) the desired reconstituted volume, in line with the guidance from the above results and such routine experimentation as might be necessary.

2. Thrombin Inhibitors

The thrombin inhibitory products may be administered to a host, for example, in the case where the drug has anti-thrombogenic activity, to obtain an anti-thrombogenic effect. In the case of larger animals, such as humans, the compounds may be administered alone or in combination with pharmaceutically acceptable diluents, excipients or carriers. The term “pharmaceutically acceptable” includes acceptability for both human and veterinary purposes, of which acceptability for human pharmaceutical use is preferred.

The salts of the disclosure may be combined and/or co-administered with any cardiovascular treatment agent. There are large numbers of cardiovascular treatment agents available in commercial use, in clinical evaluation and in pre-clinical development, which could be selected for use with a product of the disclosure for the prevention of cardiovascular disorders by combination drug therapy. Such agent can be one or more agents selected from, but not limited to several major categories, namely, a lipid-lowering drug, including an IBAT (ileal Na⁺/bile acid cotransporter) inhibitor, a fibrate, niacin, a statin, a CETP (cholesteryl ester transfer protein) inhibitor, and a bile acid sequestrant, an anti-oxidant, including vitamin E and probucol, a IIb/IIIa antagonist (e.g. abciximab, eptifibatide, tirofiban), an aldosterone inhibitor (e.g. spirolactone and epoxymexrenone), an adenosine A2 receptor antagonist (e.g. losartan), an adenosine A3 receptor agonist, a beta-blocker, acetylsalicylic acid, a loop diuretic and an ACE (angiotensin converting enzyme) inhibitor.

The salts of the disclosure may be combined and/or co-administered with any antithrombotic agent with a different mechanism of action, such as the antiplatelet agents acetylsalicylic acid, ticlopidine, clopidogrel, thromboxane receptor and/or synthetase inhibitors, prostacyclin mimetics and phosphodiesterase inhibitors and ADP-receptor (P₂T) antagonists.

The products of the disclosure may further be combined and/or co-administered with thrombolytics such as tissue plasminogen activator (natural, recombinant or modified), streptokinase, urokinase, prourokinase, anisoylated plasminogen-streptokinase activator complex (APSAC), animal salivary gland plasminogen activators, and the like, in the treatment of thrombotic diseases, in particular myocardial infarction.

The salts of the disclosure may be combined and/or co-administered with a cardioprotectant, for example an adenosine A1 or A3 receptor agonist.

There is also provided a method for treating an inflammatory disease in a patient that comprises treating the patient with a product of the disclosure and an NSAID, e.g., a COX-2 inhibitor. Such diseases include but are not limited to nephritis, systemic lupus, erythematosus, rheumatoid arthritis, glomerulonephritis, vasculitis and sarcoidosis. Accordingly, the anti-thrombotic salts of the disclosure may be combined and/or co-administered with an NSAID.

Typically, therefore, the salts described herein may be administered to a host to obtain a thrombin-inhibitory effect, or in any other thrombin-inhibitory or anti-thrombotic context mentioned herein.

Actual dosage levels of active ingredients in the pharmaceutical compositions of this disclosure may be varied so as to obtain an amount of the active compound(s) that is effective to achieve the desired therapeutic response for a particular patient, composition, and mode of administration (referred to herein as a “therapeutically effective amount”). The selected dosage level will depend upon the activity of the particular compound, the severity of the condition being treated and the condition and prior medical history of the patient being treated. However, it is within the skill of the art to start doses of the compound at levels lower than required for to achieve the desired therapeutic effect and to gradually increase the dosage until the desired effect is achieved.

According to a further aspect there is provided a parenteral formulation including a salt as described herein. The formulation may consist of the salt alone or it may contain additional components, in particular the salt may be in combination with a pharmaceutically acceptable diluent, excipient or carrier, for example a tonicity agent for the purpose of making the formulation substantially isotonic with the body of the subject to receive the formulation, e.g. with human plasma. The formulation may be in ready-to-use form or in a form requiring reconstitution prior to administration.

It is currently contemplated that, in the case of parenteral administration, for example i.v. administration, of salts of TRI 50c, the salts might for instance be administered in an amount of from 0.5 to 2.5 mg/Kg e.g. over a maximum period of 72 hours, calculated as TRI 50c. Other salts might be administered in equivalent molar amounts. The disclosure is not limited to administration in such quantities or regimens and includes dosages and regimens outside those described in the previous sentence.

Parenteral preparations can be administered by one or more routes, such as intravenous, subcutaneous, intradermal and infusion; a particular example is intravenous. A formulation disclosed herein may be administered using a syringe, injector, plunger for solid formulations, pump, or any other device recognized in the art for parenteral administration.

Liquid dosage forms for parenteral administration may include solutions, suspensions, liposome formulations, or emulsions in oily or aqueous vehicles. In addition to the active compounds, the liquid dosage forms may contain other compounds. Tonicity agents (for the purpose of making the formulations substantially isotonic with the subject's body, e.g. with human plasma) such as, for instance, sodium chloride, sodium sulfate, dextrose, mannitol and/or glycerol may be optionally added to the parenteral formulation. A pharmaceutically acceptable buffer may be added to control pH. Thickening or viscosity agents, for instance well known cellulose derivatives (e.g. methylcellulose, carboxymethylcellulose, hydroxyethylcellulose and hydroxypropylmethylcellulose), gelatin and/or acacia, may optionally be added to the parenteral formulation.

Solid dosage forms for parenteral administration may encompass solid and semi-solid forms and may include pellets, powders, granules, patches, and gels. In such solid dosage forms, the active compound is typically mixed with at least one inert, pharmaceutically acceptable excipient or carrier.

The disclosed salts may be presented as solids in finely divided solid form, for example they may be milled or micronised.

The formulations may also include antioxidants and/or preservatives. As antioxidants may be mentioned thiol derivatives (e.g. thioglycerol, cysteine, acetylcysteine, cystine, dithioerythreitol, dithiothreitol, glutathione), tocopherols, butylated hydroxyanisole, butylated hydroxytoluene, sulfurous acid salts (e.g. sodium sulfate, sodium bisulfite, acetone sodium bisulfite, sodium metabisulfite, sodium sulfite, sodium formaldehyde sulfoxylate, sodium thiosulfate) and nordihydroguaiareticacid. Suitable preservatives may for instance be phenol, chlorobutanol, benzylalcohol, methyl paraben, propyl paraben, benzalkonium chloride and cetylpyridinium chloride.

The parenteral formulations may be prepared as large volume parenterals (LVPs), e.g. larger than 100 ml, more particularly about 250 ml, of a liquid formulation of the active compound. Examples of LVPs are infusion bags. The parenteral formulations may alternatively be prepared as small volume parenterals (SVPs), e.g. about 100 ml or less of a liquid formulation of the active compound. Examples of SVPs are vials with solution, vials for reconstitution, prefilled syringes for injection and dual chamber syringe devices.

The formulations of the disclosure include those in which the salt is an alkali metal salt, for example a lithium, sodium or potassium salt, of which sodium salts may be mentioned as particular salts. Another class of formulations contains aminosugar salts of the disclosed boronic acids, for example N-methyl-D-glucamine salts. The salts mentioned in this paragraph may be administered as solutions in water, typically containing one or more additives, for example isotonicity agent(s) and/or antioxidant(s). A suitable way to store the salts is in solid form, for example as dry powder, and to make them up into solutions for administration prior to administration.

One class of formulations disclosed herein is intravenous formulations. For intravenously administered formulations, the active compound or compounds can be present at varying concentrations, with a carrier acceptable for parenteral preparations making up the remainder. Particularly, the carrier is water, particularly pyrogen free water, or is aqueous based. Particularly, the carrier for such parenteral preparations is an aqueous solution comprising a tonicity agent, for example a sodium chloride solution.

By “aqueous based” is meant that formulation comprises a solvent which consists of water or of water and water-miscible organic solvent or solvents; as well as containing a salt of disclosure in dissolved form, the solvent may have dissolved therein one or more other substances, for example an antioxidant and/or an isotonicity agent. As organic cosolvents may be mentioned those water-miscible solvents commonly used in the art, for example propyleneglycol, polyethyleneglycol 300, polyethyleneglycol 400 and ethanol. Preferably, organic co-solvents are only used in cases where the active agent is not sufficiently soluble in water for a therapeutically effective amount to be provided in a single dosage form. As previously indicated, the disclosure includes formulations of alkali metal salts of the disclosed boronic acids, e.g. TRI 50c, having a solvent which consists of water.

The solubility of the active compound in the present formulations may be such that the turbidity of the formulation is lower than 50 NTU, e.g. lower than 20 NTU such as lower than 10 NTU.

It is desirable that parenteral formulations are administered at or near physiological pH. It is believed that administration in a formulation at a high pH (i.e., greater than 8) or at a low pH (i.e., less than 5) is undesirable. In particular, it is contemplated that the formulations would be administered at a pH of between 6.0 and 7.0 such as a pH of 6.5.

The parenteral formulation may be purged of air when being packaged. The parenteral formulation may be packaged in a sterile container, e.g. vial, as a solution, suspension, gel, emulsion, solid or a powder. Such formulations may be stored either in ready-to-use form or in a form requiring reconstitution prior to administration.

Parenteral formulations according to the disclosure may be packaged in containers. Containers may be chosen which are made of material which is non-reactive or substantially non-reactive with the parenteral formulation. Glass containers or plastics containers, e.g. plastics infusion bags, may be used. A concern of container systems is the protection they afford a solution against UV degradation. If desired, amber glass employing iron oxide or an opaque cover fitted over the container may afford the appropriate UV protection.

Plastics containers such as plastics infusion bags are advantageous in that they are relatively light weight and non-breakable and thus more easily stored. This is particularly the case for Large Volume parenterals.

The intravenous preparations may be prepared by combining the active compound or compounds with the carrier. After the formulation is mixed, it may be sterilized, for example using known methods. Once the formulation has been sterilized, it is ready to be administered or packaged, particularly in dark packaging (e.g. bottles or plastics packaging), for storage. It is envisaged, however, that the disclosed salts might not be stored in solution but as dry solids, particularly a finely divided form such as, for example, a lyophilisate, in order to prolong shelf life; this would of course apply to other parenteral formulations, not only intravenous ones.

The intravenous preparations may take the form of large volume parenterals or of small volume parenterals, as described above.

In a specific embodiment, the present disclosure is directed to products, particularly kits, for producing a single-dose administration unit. The products (kits) may each contain both a first container having the active compound (optionally combined with additives, for example anti-oxidant, preservative and, in some instances, tonicity agent) and a second container having the carrier/diluent (for example water, optionally containing one or more additives, for example tonicity agent). As examples of such products may be mentioned single and multi-chambered (e.g. dual-chamber) pre-filled syringes; exemplary pre-filled syringes are available from Vetter GmbH, Ravensburg, Germany. Such dual chamber syringes or binary syringes will have in one chamber a dry preparation including or consisting of the active compound and in another chamber a suitable carrier or diluent such as described herein. The two chambers are joined in such a way that the solid and the liquid mix to form the final solution.

One class of formulations disclosed herein comprises subcutaneous or intradermal formulations (for example formulations for injection) in which the active salt (or active agent combination) is formulated into a parenteral preparation that can be injected subcutaneously or intradermally. The formulation for administration will comprise the active salt and a liquid carrier.

The carrier utilized in a parenteral preparation that will be injected subcutaneously or intradermally may be an aqueous carrier (for example water, typically containing an additive e.g. an antioxidant and/or an isotonicity agent) or a nonaqueous carrier (again one or more additives may be incorporated). As a non-aqueous carrier for such parenteral preparations may be mentioned highly purified olive oil.

The active compound and the carrier are typically combined, for example in a mixer. After the formulation is mixed, it is preferably sterilized, such as with U.V. radiation. Once the formulation has been sterilized, it is ready to be injected or packaged for storage. It is envisaged, however, that the disclosed salts will not be stored in liquid formulation but as dry solids, in order to prolong shelf life.

For making subcutaneous implants, the active salt may suitably be formulated together with one or more polymers that are gradually eroded or degraded when in use, e.g. silicone polymers, ethylene vinylacetate, polyethylene or polypropylene.

Transdermal formulations may be prepared in the form of matrices or membranes, or as fluid or viscous formulations in oil or hydrogels or as a compressed powder pellet. For transdermal patches, an adhesive which is compatible with the skin may be included, such as polyacrylate, a silicone adhesive or polyisobutylene, as well as a foil made of, e.g., polyethylene, polypropylene, ethylene vinylacetate, polyvinylchloride, polyvinylidene chloride or polyester, and a removable protective foil made from, e.g., polyester or paper coated with silicone or a fluoropolymer. For the preparation of transdermal solutions or gels, water or organic solvents or mixtures thereof may be used. Transdermal gels may furthermore contain one or more suitable gelling agents or thickeners such as silicone, tragacanth, starch or starch derivatives, cellulose or cellulose derivatives or polyacrylic acids or derivatives thereof. Transdermal formulations may also suitably contain one or more substances that enhance absorption though the skin, such as bile salts or derivatives thereof and/or phospholipids. Transdermal formulations may be prepared according to a method disclosed in, e.g., B W Barry, “Dermatological Formulations, Percutaneous Absorption”, Marcel Dekker Inc., New York-Basel, 1983, or Y W Chien, “Transdermal Controlled Systemic Medications”, Marcel Dekker Inc., New York-Basel, 1987.

It will be understood from the aforegoing that there are provided pharmaceutical products comprising an alkali metal salt, particularly sodium salt, of a boronic acid of Formula (I) in dry fine particle form, suitable for reconstitution into an aqueous read-to-use parenteral formulation. The alkali metal salt is suitably an acid salt. The alkali metal salt may be in a small volume parenteral unit dosage form. The alkali metal salt may be presented in a form, e.g. dry powder form, suitable for reconstituting as a large volume parenteral. One example is a sodium salt of a boronic acid of Formula (I), particularly TRI 50c, in dry powder form for reconstitution as a liquid intravenous formulation (solution) containing a tonicity agent, particularly sodium chloride. The dry powder form of a salt used in a parenteral formulation may be a lyophilisate. The reconstituted solution may be administered by injection or infusion.

In the case of oral administration, the compounds, particularly the salts of amino- or peptido-boronic acids, may be administered in a form which prevents the salt from contact with the acidic gastric juice, such as enterically coated formulations, which thus prevent release of the salt until it reaches the duodenum.

The enteric coating is suitably made of carbohydrate polymers or polyvinyl polymers, for example. Examples of enteric coating materials include, but are not limited to, cellulose acetate phthalate, cellulose acetate succinate, cellulose hydrogen phthalate, cellulose acetate trimellitate, ethyl cellulose, hydroxypropyl-methylcellulose phthalate, hydroxypropylmethylcellulose acetate succinate, carboxymethyl ethylcellulose, starch acetate phthalate, amylose acetate phthalate, polyvinyl acetate phthalate, polyvinyl butyrate phthalate, styrene-maleic acid copolymer, methyl-acrylate-methacrylic acid copolymer (MPM-05), methylacrylate-methacrylic acid-methylmethacrylate copolymer (MPM-06), and methylmethacrylate-methacrylic acid co-polymer (Eudragit® L & S). Optionally, the enteric coating contains a plasticiser. Examples of the plasticiser include, but are not limited to, triethyl citrate, triacetin, and diethyl phthalate.

It is currently contemplated that, in the case of oral administration of salts of TRI 50c, the salts might for instance be administered in an amount of from 0.5 to 2.5 mg/Kg twice daily, calculated as TRI 50c. Other salts might be administered in equivalent molar amounts. However, the presently described methods are not limited to administration in such quantities or regimens and includes dosages and regimens outside those described in the previous sentence.

According to a further aspect there is provided an oral pharmaceutical formulation including a product as described herein, in admixture with a pharmaceutically acceptable adjuvant, diluent or carrier.

Solid dosage forms for oral administration include capsules, tablets (also called pills), powders and granules. In such solid dosage forms, the active compound is typically mixed with at least one inert, pharmaceutically acceptable excipient or carrier such as sodium citrate or dicalcium phosphate and/or one or more: a) fillers or extenders such as starches, lactose, sucrose, glucose, mannitol and silicic acid; b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules and tablets, the dosage form may also comprise buffering agents. Solid compositions of a similar type may also be employed as fillers in soft and hard-filled gelatin capsules using such excipients as lactose or milk sugar as well as high molecular weight polyethylene glycol, for example.

Suitably, the oral formulations may contain a dissolution aid. The dissolution aid is not limited as to its identity so long as it is pharmaceutically acceptable. Examples include nonionic surface active agents, such as sucrose fatty acid esters, glycerol fatty acid esters, sorbitan fatty acid esters (e.g., sorbitan trioleate), polyethylene glycol, polyoxyethylene hydrogenated castor oil, polyoxyethylene sorbitan fatty acid esters, polyoxyethylene alkyl ethers, methoxypolyoxyethylene alkyl ethers, polyoxyethylene alkylphenyl ethers, polyethylene glycol fatty acid esters, polyoxyethylene alkylamines, polyoxyethylene alkyl thioethers, polyoxyethylene polyoxypropylene copolymers, polyoxyethylene glycerol fatty acid esters, pentaerythritol fatty acid esters, propylene glycol monofatty acid esters, polyoxyethylene propylene glycol monofatty acid esters, polyoxyethylene sorbitol fatty acid esters, fatty acid alkylolamides, and alkylamine oxides; bile acid and salts thereof (e.g., chenodeoxycholic acid, cholic acid, deoxycholic acid, dehydrocholic acid and salts thereof, and glycine or taurine conjugate thereof); ionic surface active agents, such as sodium laurylsulfate, fatty acid soaps, alkylsulfonates, alkylphosphates, ether phosphates, fatty acid salts of basic amino acids; triethanolamine soap, and alkyl quaternary ammonium salts; and amphoteric surface active agents, such as betaines and aminocarboxylic acid salts.

The active compounds may also be in micro-encapsulated form, if appropriate, with one or more of the above-mentioned excipients.

Liquid dosage forms for oral administration include pharmaceutically acceptable emulsions, solutions, suspensions, syrups and elixirs. In addition to the active compounds, the liquid dosage forms may contain inert diluents commonly used in the art such as water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan and mixtures thereof. Besides inert diluents, the oral compositions may also include adjuvants such as wetting agents, emulsifying and suspending agents, sweetening, flavouring and perfuming agents. Suspensions, in addition to the active compounds, may contain suspending agents such as ethoxylated isostearyl alcohols, polyoxyethylene sorbitol and sorbitan esters, microcrystalline cellulose, aluminium metahydroxide, bentonite, agar-agar, and tragacanth and mixtures thereof.

The presently disclosed product may be presented as solids in finely divided solid form, for example they may be micronised. Powders or finely divided solids may be encapsulated.

The active compound may be given as a single dose, in multiple doses or as a sustained release formulation.

It will be understood from the aforegoing that there are provided pharmaceutical products comprising an alkaline earth metal salt, particularly calcium salt, of a boronic acid of Formula (IIIa) in dry fine particle form, suitable for oral administration. The alkaline earth metal salt is suitably an acid salt.

3. Proteasome Inhibitors

The proteasome inhibitors of the disclosure may in particular be administered intravenously as described above. In embodiments, products containing N-(2-pyrazine) carbonyl-phenylalanine-leucine boronic acid (e.g. the monosodium or hemicalcium salt thereof) may be packaged in unit doses of containing a molar amount corresponding to the molar quantity of 3.5 mg of the free acid.

The disclosure includes pharmaceutical formulation, whether in ready-to-use form or in a form requiring reconstitution prior to administration, adapted for intravenous administration and comprising the reaction product obtained by combining a pharmaceutically acceptable base with a boronic acid of formula (XXX), e.g. N-(2-pyrazine) carbonyl-phenylalanine-leucine boronic acid.

To be mentioned are lyophilisates of a pharmaceutically acceptable base addition salt of compounds of Formula (XXX), e.g. the compound N-(2-pyrazine) carbonyl-phenylalanine-leucine boronic acid.

A further aspect of the disclosure resides in a method of storing an organoboronic acid of formula (XXX) for a period of at least six months, comprising providing the acid in the form of a reaction product thereof with a pharmaceutically acceptable base in a sealed container and storing it for at least six months at a temperature of at least 0° C.

A product of the disclosure comprises a package comprising:
(i) a sealed container containing a boronic acid of formula (XXX) in the form of a reaction product thereof with a pharmaceutically acceptable base; and
(ii) instructions permitting the container to be stored at a temperature of 10° C. or more for a period of 8 months or more, e.g. at a temperature of 15° C. or more for a period of 12 months or more.
   4. Other Boronic Acid Drugs

Other boronic acid drugs may be formulated as appropriate for oral or parenteral administration, for example as indicated above.

### EXAMPLES

### Examples 1 to 4

### Introductory Remarks

Apparatus

Throughout the following procedures of Examples 1 to 4, standard laboratory glassware and, where appropriate, specialised apparatus for handling and transferring of air sensitive reagents are used.

All glassware is heated at 140-160° C. for at least 4 hours before use and then cooled either in a desiccator or by assembling hot and purging with a stream of dry nitrogen.

Solvents

The organic solvents used in the procedures of Examples 1 to 4 are all dry. Suitably, they are dried over sodium wire before use.

Dryness

In the drying procedures of Example 1 to 4, products are tested for dryness (including dryness in terms of organic solvent) by observing weight loss on drying. The following procedure was followed to determine loss on drying: a sample was placed in a vacuum drier and dried at 40° C. at 100 mbar for 2 hours. Products are considered dry when the decrease in weight upon drying is less than 0.5% of the total weight of the starting material.

Examples 1 to 4 describe performance of the following reaction scheme and conversion of the resultant TRI 50c to sodium and calcium salts thereof:

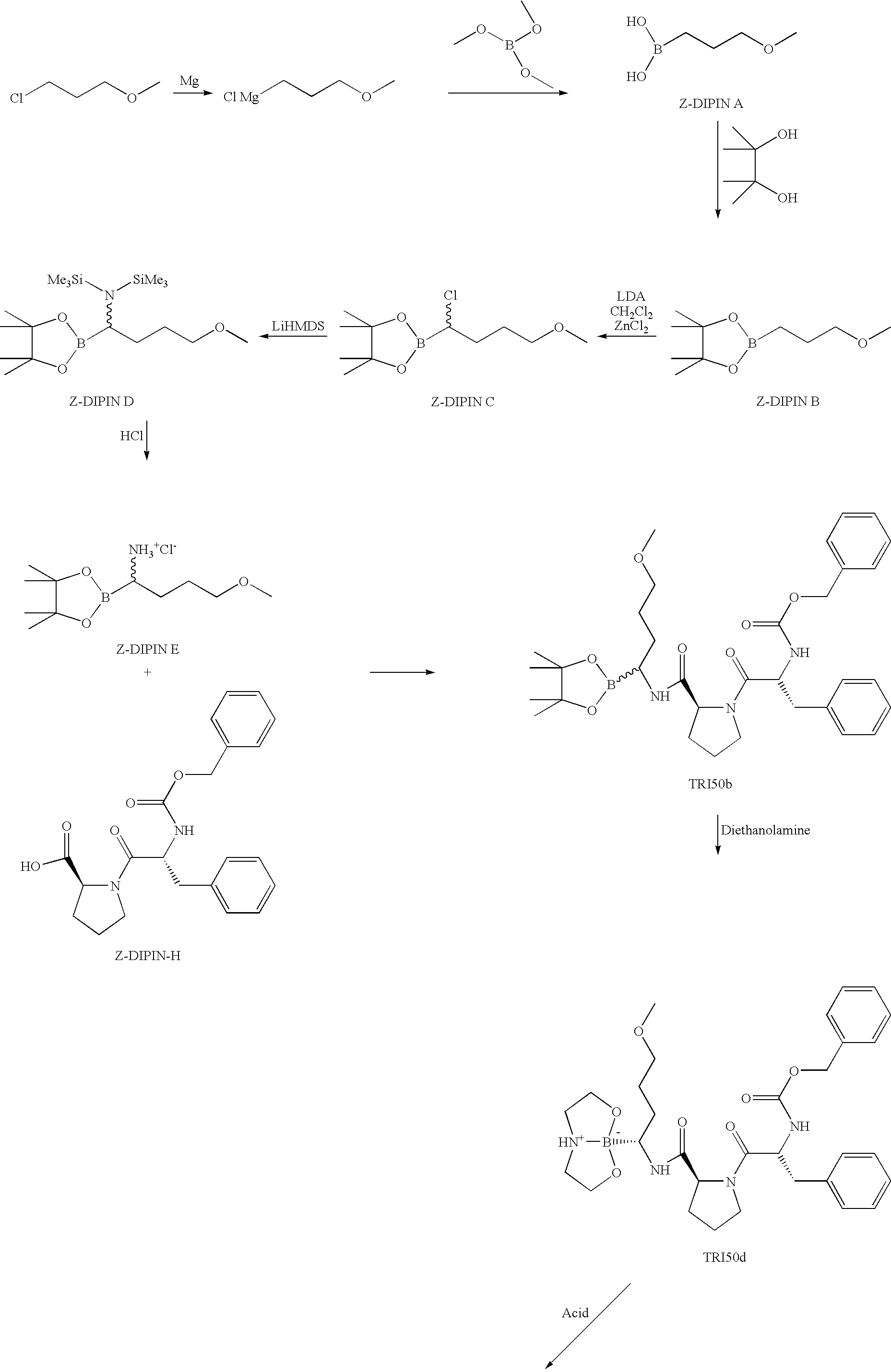

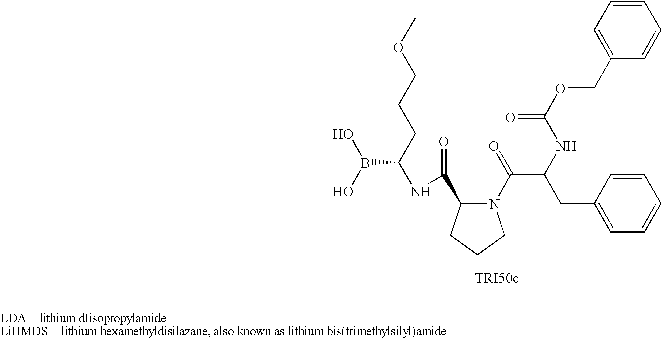

### Example 1

### Synthesis of TRI 50B

Step 1: Z-DIPIN B

Procedure A

17.8 g (732.5 mmole) magnesium turnings, 0.1 g (0.4 mmole) iodine and 127 ml dry tettahydrofuran are charged and heated to reflux. Then 15 ml of a solution of 66 g (608 mmole) 1-chloro-3-methoxypropane in 185 ml dry tetrahydrofuran are added and stirred under reflux until the vigorous reaction starts. After the initial exotherm ceases, the solution of 1-chloro-3-methoxypropane is added slowly to maintain gentle reflux until all the magnesium is consumed. After the reaction is finished, the reaction mixture is cooled to ambient temperature and slowly added to a solution of 64.4 g (620 mmole) trimethylborate in 95 ml dry tetrahydrofuran; the latter solution is cooled to below 0° C. and, if it warms up during the course of the reaction, the reaction mixture must be added to it sufficiently slowly to maintain the temperature of this solution below 65° C. Upon complete addition, the reaction mixture is allowed to warm to about 0° C. and stirred for another 60 minutes. Then a solution of 22.4 ml sulfuric acid in 400 ml water is added slowly so as to maintain the temperature below 20° C. The layers are allowed to settle and the phases are separated. The aqueous layer is rewashed three times with 200 ml tert.-butylmethylether. The combined organic layers are allowed to settle and additional water separated from this solution is removed. The organic layer is dried over magnesium sulfate, filtered and evaporated to dryness. The evaporation residue is filtered from the precipitated solid and the filtrate dissolved in 175 ml toluene. 34.8 g (292 mmole) pinacol is charged to the solution followed by stirring at ambient temperature for not less than 10 hours. The solution is evaporated to dryness, dissolved in 475 ml n-heptane and washed three times with 290 ml saturated aqueous solution of sodium hydrogen carbonate. The n-heptane solution is evaporated to dryness and the evaporation residue distilled and the fraction with Bp 40-50° C. at 0.1-0.5 mbar recovered.

Boiling point: 40-50° C./0.1-0.5 mbar

Yield: 40.9 g (70%) Z-DIPIN B (oil)

Procedure B

17.8 g (732.5 mmole) magnesium turnings, 0.1 g (0.4 mmole) iodine and 127 ml dry tetrahydrofuran are charged and heated to reflux. Then 15 ml of a solution of 66 g (608 mmole) 1-chloro-3-methoxypropane in 185 ml dry tetrahydrofuran are added and stirred under reflux until the vigorous reaction starts. After the initial exotherm ceases; the solution of 1-chloro-3-methoxypropane is added slowly to maintain gentle reflux. After the reaction is finished, the reaction mixture is cooled to ambient temperature and slowly added to a solution of 64.4 g (620 mmole) trimethylborate in 95 ml dry tetrahydrofuran, maintaining the temperature of this solution below minus 65° C. Upon complete addition, the reaction mixture is allowed to warm to about 0° C and stirred for another 60 minutes. Then a solution of 22.4 ml sulfuric acid in 400 ml water is added slowly so as to maintain the temperature below 20° C. The organic solvent is removed by distillation under vacuum. 300 ml n-heptane is charged to the aqueous solution of the evaporation residue followed by addition of 34.8 g (292 mmole) pinacol. The two-phase-mixture is stirred at ambient temperature for not less than 2 hours. After allowing the layers to settle, the aqueous phase is separated. 300 ml n-heptane is charged to the aqueous solution and the two-phase-mixture is stirred at ambient temperature for not less than 2 hours. After allowing the layers to settle, the aqueous phase is separated. The organic layers are combined and washed once with 200 ml water, followed by 200 ml saturated sodium hydrogen carbonate solution and two further washes with 200 ml water each. The n-heptane solution is evaporated to dryness and the evaporation residue distilled and the fraction with Bp 40-50° C. at 0.1-0.5 mbar recovered.

Boiling point: 40-50° C./0.1-0.5 mbar

Yield: 40.9 g (70-85%) Z-DIPIN B (oil)

Step 2: Z-DIPIN C

16.6 g (164 mmole) diisopropylamine and 220 ml tetrahydrofuran are charged and cooled to −30 to −40° C. To this solution 41.8 g (163 mmole) n-butyl lithium, 25% in n-heptane is added, followed by stirring at 0 to −5° C. for one hour. This freshly prepared solution of lithium diisopropylamide is cooled to −30° C. and then added to a solution of 27.9 g (139 mmole) Z-DIPIN B in 120 ml tetrahydrofuran and 35.5 g (418 mmole) dichloromethane at a temperature between −60 and −75° C. The solution is stirred at that temperature for half an hour followed by addition of 480 ml (240 mmole) 0.5N anhydrous Zinc(II)-chloride in tetrahydrofuran or 32.5 g (240 mmole) anhydrous solid Zinc(II)-chloride. After stirring at −65° C. for one hour, the reaction mixture is allowed to warm to ambient temperature and stirred for another 16-18 hours. The reaction mixture is evaporated to dryness (i.e. until solvent is removed) and followed by addition of 385 ml n-heptane. The reaction mixture is washed with 150 ml 5% sulfuric acid, with 190 ml saturated sodium hydrogen carbonate solution, and 180 ml saturated sodium chloride solution. The organic layer is dried over magnesium sulfate, filtered and evaporated to dryness (i.e. until solvent is removed). The oily residue is transferred into the next step without further purification.

Yield: 19 g (55%) Z-DIPIN C

Step 3: Z-DIPIN D

To a solution of 23.8 g (148 mmole) hexamethyldisilazane in 400 ml tetrahydrofuran at −15° C. is added 34.7 g (136 mmole) n-butyl lithium, 25% in n-heptane and stirred for one hour. The solution is cooled to −55° C. followed by the addition of 30.6 g (123 mmole) Z-DIPIN C dissolved in 290 ml tetrahydrofuran and 35 ml tetrahydrofuran to this freshly prepared solution of LIHMDS. The solution is allowed to warm to ambient temperature and stirred for 12 hours. The reaction mixture is evaporated to dryness, the evaporation residue dissolved in 174 ml n-heptane, washed with 170 ml water and 75 ml saturated sodium chloride solution. The organic phase is dried over magnesium sulfate, filtered and evaporated to complete dryness (i.e. until solvent is removed). The oily residue is dissolved in 100 g n-heptane. This solution is carried over into the next step without further purification.

Yield: 32.2 g (70%) Z-DIPIN D

Step 4: Z-DIPIN (TRI50b, Crude)

A solution of 26.6 g (71 mmole) Z-DIPIN D in 82.6 g n-heptane is diluted with 60 ml n-heptane and cooled to −60° C. followed by introduction of 10.5 g (285 mmole) hydrogen chloride. The reaction mixture is subsequently evacuated and flushed with nitrogen, while the temperature is increased in increments of about 20° C. to ambient temperature. The solvent is removed from the oily precipitate and replaced several times by 60 ml fresh n-heptane. The oily residue is dissolved in 60 ml tetrahydrofuran (Solution A).

To a different flask 130 ml tetrahydrofuran, 24.5 g (61.5 mmole) Z-D-Phe-Pro-OH and 6.22 g (61.5 mmole) N-methylmorpholine are charged and cooled to −20° C. To this solution a solution of 8.4 g (61.5 mmole) isobutylchloroformate in 20 ml tetrahydrofuran is added and stirred for 30 minutes, followed by addition of Solution A at −25° C. Upon complete addition, up to 16 ml (115 mmole) triethylamine is added to adjust the pH to 9-10, measured using a pH stick. The reaction mixture is allowed to warm to ambient temperature and stirred for 3 hours, still under nitrogen. The solvent is evaporated to dryness and the evaporation residue dissolved in 340 ml tert.-butylmethylether (t-BME). The solution of Z-DIPIN in t-BME is washed twice with 175 ml 1.5% hydrochloric acid. The combined acidic washes are given a rewash with 175 ml t-BME. The combined organic layers are washed with 175 ml water, with 175 ml saturated sodium hydrogen carbonate solution, with 175 ml 25% sodium chloride solution, dried over magnesium sulfate and filtered. This solution is carried over into the next step without further purification.

Yield: 29.9 g (80%) Z-DIPIN

### Example 2

### Synthesis of TRI 50D (Diethanolamine Adduct of TRI 50C)

The starting material used in this Example is the solution of TRI 50b (“Z-DIPIN”) obtained in Example 1. The solution is carried forward to the synthesis of TRI 50d without further purification. The solution of Z-DIPIN in t-BME (containing 7.0 g (11.5 mmole) (R,S,R) TRI50b, calculated based on HPLC results of Z-DIPIN) is evaporated to dryness and the evaporation residue dissolved in 80 ml diethylether. 1.51 g (14.4 mmole) diethanolamine is added and the mixture heated at reflux for at least 10 hours, during which process the product precipitates. The suspension is cooled to 5-10° C., filtered and the filter residue washed with diethylether.

To improve chiral and chemical purity the wet filter cake (7 g) is dissolved in 7 ml dichloromethane, cooled to 0-5° C. and the product precipitated by addition of 42 ml diethylether and filtered. The isolated wet product is dried at 35° C. in vacuum or at least 4 hours, until day.

Yield: 5.5 g (80%) Tri50d

Melting Point: 140-145° C.

### Example 3

### Preparation of Sodium Salt of TRI50C

1.5 kg (2.5 mole) TRI50d from Example 2 is dissolved in 10.5 L dichloromethane. 11 L 2% hydrochloric acid is added and the mixture is stirred for at most 30 minutes (optimally about 20 minutes) at room temperature. A precipitate forms in the organic phase. After stirring, the layers are allowed to settle and separated. The aqueous layer is rewashed twice with 2.2 L dichloromethane. The combined organic layers are washed with a solution of 625 g ammonium chloride in 2.25 L water. (The ammonium chloride buffers the pH of the aqueous extractions to be within a range of from about pH 1-2 to about pH 4-5, as strongly acidic conditions might cleave peptide bonds). The organic phase is dried over magnesium sulfate, filtered and the filtrate evaporated to dryness. An assay of the free boronic acid is performed (by the RP HPLC method of Example 38 for at most 30 mins (optionally about 20 min) at room temperature) and the amounts of the solvents and base for conversion of the acid to the salt are calculated. If 2.5 mol of the free acid is obtained, the evaporation residue is dissolved in 5 L acetonitrile followed by addition of a solution of 100 g (2.5 mole) sodium hydroxide as a 5% solution in 2.2 L water. The solution is stirred for two hours at ambient temperature (e.g. 15-30° C., optimally room temperature) and then evaporated in vacuum (of ca. 10 mmHg) at a temperature not exceeding 35° C. The evaporation residue is repeatedly dissolved in 3.5 L fresh acetonitrile and evaporated to dryness to remove traces of water. If the evaporation residue is dry, it is dissolved in 3 L acetonitrile (or alternatively in 6 L THF) and slowly added to a mixture of 32 L n-heptane and 32 L diethylether. The addition is performed slowly enough to avoid lumping or sticking of the product and is carried out over a period of not less than 30 minutes. The precipitated product is filtered off, washed with n-heptane and dried under vacuum at a temperature initially of about 10° C. and then increasing to a limit of about 35° C., until dry.

Yield: 1.0 kg (70%) Tri50c sodium salt.

### Example 4

### Preparation of Calcium Salt of TRI50C

1.5 kg (2.5 mole) TRI50d from Example 2 is dissolved in 10.5 L dichloromethane. 11 L 2% hydrochloric acid is added and the mixture is stirred for at most 30 minutes (optimally about 20 minutes) at room temperature. After stirring the layers are allowed to settle and separated. The aqueous layer is given a rewashed twice with 2.2 L dichloromethane. The combined organic layers are washed with a solution of 625 g ammonium chloride in 2.25 L water. The organic phase is dried over magnesium sulfate, filtered and the filtrate evaporated to dryness. An assay of the free boronic acid is performed and the amounts of the solvents and base for conversion of the acid to the salt are calculated. If 2.5 mol of the free acid is obtained, the evaporation residue is dissolved in 5 L acetonitrile followed by addition of a suspension of 93 g (1.25 mole) calcium hydroxide in 1 L water. The solution is stirred for two hours at ambient temperature (e.g. 15-30° C., optimally room temperature) and then evaporated under vacuum (of ca. 10 mmHg) at a temperature initially of about 10° C. and then increasing to a limit of about 35° C. The evaporation residue is repeatedly dissolved in 3.5 L fresh acetonitrile and evaporated to dryness to remove traces of water. If the evaporation residue is dry, it is dissolved in 6 L tetrahydrofuran and slowly added to a mixture of 32 L n-heptane and 32 L diethylether. The addition is performed slowly enough to avoid lumping or sticking of the product and is carried out over a period of not less than 30 minutes. The precipitated product is filtered off, washed with n-heptane and dried under vacuum (of ca. 10 mmHg) at a temperature below 35° C. until dry.

Yield: 0.98 kg (70%) Tri50c calcium salt.

The procedures of Examples 1 to 4 may be scaled up and, if operated carefully, will produce highly pure salts. In the diethanolamine precipitation step it is important to use 1.25 equivalents of diethanolamine per equivalent of (R,S,R) TRI 50b. In the hydrolysis of the diethanolamine ester, it is important to avoid excessively long contact with the aqueous acid. Likewise the TRI 50b should be synthesised via the Grignard reaction to Z-DIPIN A.

### Example 5

### Alternative Conversion of TRI 50B to TRI 50C

The synthetic procedures described in this and subsequent synthetic examples were generally, performed under nitrogen and using dry solvents as supplied from commercial sources.

1. Approximately 300 g of TRI 50b, obtained by the HPLC purification of racemic TRI 50b) were dissolved in approximately 2.5 L diethylether. It is estimated that different batches of TRI 50b had isomeric purities ranging from 85% R,S,R to in excess of 95% R,S,R.

2. Approximately 54 ml diethanolamine were added (1:1 stoichiometry with total TRI 50b content), and the mixture was refluxed at 40° C.

3. The precipitated product was removed, washed several times with diethylether and dried.

4. The dry product was dissolved in CHCl₃. Hydrochloric acid (pH 1) was added and the mixture was stirred approximately 1 h at room temperature.

5. The organic layer was removed and washed with NH₄Cl solution.

6. The organic solvent was distilled off and the residual solid product was dried.

Typical yield: Approximately 230 g

### Example 6

### Preparation of Lithium Salt of TRI50C

Cbz-Phe-Pro-BoroMpg-OH obtained by the method of Example 5 (20.00 g, 38.1 mM) is dissolved in acetonitrile (200 ml) with stirring at room temperature. To this solution is added LiOH as a 0.2M solution in distilled water (190 ml). The resultant clear solution is stirred for 2 hours at room temperature and then evacuated to dryness under vacuum with its temperature not exceeding 37° C. The resultant oil/tacky liquid is redissolved in 500 ml distilled water necessary with light warming for about 20 minutes. The solution is filtered through filter paper and evacuated to dryness, again with the temperature of the solution not exceeding 37° C. The resultant product is dried under vacuum overnight to normally yield a white brittle solid.

The salt was then dried under vacuum over silica to constant weight (72 h).

Yield 17.89 g.

Microanalysis:

| C % Found | H % Found | N % Found | B % Found | Metal % Found |
|---|---|---|---|---|
| (Calc.) | (Calc.) | (Calc.) | (Calc.) | (Calc.) |
| 57.14 | 6.60 | 7.34 | 2.07 | Li 1.26 |
| (61.03) | (6.64) | (7.90) | (2.03) | (1.31) |

### Example 7

### UV/Visible Spectra of Lithium Salt of TRI50C

UV/Visible spectra of the salt resulting from the procedure of Example 6 were recorded in distilled water at 20° C. from 190 nm to 400 nm. The salt gave λₘₐₓ at 210 and 258 nm. The weight of the dried salt was then measured for the purposes of calculating the extinction coefficient. The λₘₐₓ at 258 nm was used. The extinction coefficient was calculated using the formula:—

A=εcl where A is the absorbance
C is the concentration
I the path length of the UV cell
and ε is the extinction coefficient.

Extinction coefficient: 451

### Example 8

### Aqueous Solubility of Lithium Salt of TRI50C

The salt used in this Example was made using a modification of the process described in Example 6. The modified process differs from that described in that 100 mg of TRI 50c was used as starting material, the product of the redissolution in water was dried by freeze drying and the filtration was carried out through a 0.2 μm filter. The salt is believed to contain about 85% of R,S,R isomer.

To determine maximum aqueous solubility 25 mg of the dried salt were shaken in water at 37° C., the sample filtered and the UV spectrum measured. The salt left a white residue of undissolved material. The lithium salt was comparatively soluble and so was redissolved at 50 mg/ml in the same manner previously described.

Solubility when dissolved at 25 mg/ml: 43 mM (23 mg/ml).

Solubility when dissolved at 50 mg/ml: 81 mM (43 mg/ml).

### Example 9

### Preparation of Sodium Salt of TRI50C

Cbz-Phe-Pro-BoroMpg-OH obtained by the method of Example 5 (20.00 g, 38.1 mM) is dissolved in acetonitrile (200 ml) with stirring at room temperature. To this solution is added NaOH as a 0.2M solution in distilled water (190 ml). The resultant clear solution is stirred for 2 hours at room temperature and then evacuated to dryness under vacuum with its temperature not exceeding 37° C. The resultant oil/tacky liquid is redissolved in 500 ml distilled water with light warming for about 15-20 minutes. The solution is filtered through filter paper and evacuated to dryness, again with the temperature of the solution not exceeding 37° C. The resultant product is dried under vacuum overnight to normally yield a white brittle solid. The product may be present as an oil or tacky solid due to residual water, in which case it is dissolved in ethyl acetate and evacuated to dryness to produce the product as a white solid.

The salt was then dried under vacuum over silica to constant weight (72 h).

Yield: Over 50%.

Microanalysis:

| C % Found | H % Found | N % Found | B % Found | Metal % Found |
|---|---|---|---|---|
| (Calc.) | (Calc.) | (Calc.) | (Calc.) | (Calc.) |
| 59.93 | 6.47 | 7.31 | 1.91 | Na 3.81 |
| (59.24) | (6.44) | (7.67) | (1.98) | (4.20) |

### Example 10

### UV/Visible Spectra of Sodium Salt of TRI50C

UV/Visible spectra of the sodium salt resulting from the procedure of Example 9 were recorded in distilled water at 20° C. from 190 nm to 400 nm. The salt gave λₘₐₓ at 210 and 258 nm. The weight of the dried salt was then measured for the purposes of calculating the extinction coefficient. The λₘₐₓ at 258 nm was used. The extinction coefficient was calculated using the formula:—

A=εcl where A is the absorbance
C is the concentration
I the path length of the UV cell
and ε is the extinction coefficient.

Extinction coefficient: 415.

### Example 11

### Aqueous Solubility of Sodium Salt of TRI50C

The salt used in this Example was made using a modification of the process described in Example 9. The modified process differs from that described in that 100 mg of TRI 50c was used as starting material, the product of the redissolution in water was dried by freeze drying and the filtration was carried out through a 0.2 μm filter. The salt is believed to contain about 85% of R,S,R isomer.

To determine maximum aqueous solubility 25 mg of the dried salt were shaken in water at 37° C., the sample filtered and the UV spectrum measured. The salt left a white residue of undissolved material. The sodium salt was comparatively soluble and so was redissolved at 50 mg/ml in the same manner previously described.

Solubility when dissolved at 25 mg/ml: 44 mM (25 mg/ml).

Solubility when dissolved at 50 mg/ml: 90 mM (50 mg/ml).

### Example 12

### Preparation of Potassium Salt of TRI50C

Cbz-Phe-Pro-BoroMpg-OH obtained by the method of Example 5 (20.00 g, 38.1 mM) is dissolved in acetonitrile (200 ml) with stirring at room temperature. To this solution is added KOH as a 0.2M solution in distilled water (190 ml). The resultant clear solution is stirred for 2 hours at room temperature and then evacuated to dryness under vacuum with its temperature not exceeding 37° C. The resultant oil/tacky liquid is redissolved in 1L distilled water with warming to 37° C. for about 2 hours. The solution is filtered through filter paper and evacuated to dryness, again with the temperature of the solution not exceeding 37° C. The resultant product is dried under vacuum overnight to normally yield a white brittle solid.

Yield: 14.45 mg.

The salt was then dried under vacuum over silica to constant weight (72 h).

Microanalysis:

| C % Found | H % Found | N % Found | B % Found | Metal % Found |
|---|---|---|---|---|
| (Calc.) | (Calc.) | (Calc.) | (Calc.) | (Calc.) |
| 54.84 | 6.25 | 7.02 | 2.01 | K 4.29 |
| (57.55) | (6.26) | (7.45) | (1.92) | (6.94) |

### Example 13

### UV/Visible Spectra of Potassium Salt of TRI50C

UV/Visible spectra of the potassium salt resulting from the procedure of Example 12 were recorded in distilled water at 20° C. from 190 nm to 400 nm. TRI50C and the salt gave λₘₐₓ at 210 and 258 nm. The weight of the dried salt was then measured for the purposes of calculating the extinction coefficient. The λₘₐₓ at 258 nm was used. The extinction coefficient was calculated using the formula:—

A=εcl where A is the absorbance
C is the concentration
I the path length of the UV cell
and ε is the extinction coefficient.

Extinction coefficient: 438.

### Example 14

### Aqueous Solubility of Potassium Salt of TRI50C

The salt used in this Example was made using a modification of the process described in Example 12. The modified process differs from that described in that 100 mg of TRI 50c was used as starting material, the product of the redissolution in water was dried by freeze drying and the filtration was carried out through a 0.2 μm filter. The salt is believed to contain about 85% of R,S,R isomer.

To determine maximum aqueous solubility 25 mg of the dried salt were shaken in water at 37° C., the sample filtered and the UV spectrum measured. The salt left a white residue of undissolved material.

Solubility when dissolved at 25 mg/ml: 29 mM (16 mg/ml).

### Example 15

### Preparation of Zinc Salt of TRI 50C

The relative solubility of zinc hydroxide is such that, if the hydroxide had been used to prepare the corresponding TRI 50c salt using the procedure of Example 6, they would not have resulted in homogeneous salt formation. A new method was therefore developed to prepare the zinc salt, as described in this and the next examples.

TRI 50c sodium salt (2.24 g, 4.10 mM) was dissolved in distilled water (100 ml) at room temperature and zinc chloride in THF (4.27 ml, 0.5M) was carefully added with stirring. A white precipitate that immediately formed was filtered off and washed with distilled water. This solid was dissolved in ethyl acetate and washed with distilled water (2×50 ml). The organic solution was evacuated to dryness and the white solid produced dried over silica in a desiccator for 3 days before microanalysis. Yield 1.20 g.

¹H NMR 400 MHz, δ_{H}(CD₃OD) 7.23-7.33 (20H, m, ArH), 5.14 (4H, m, PhCH₂O), 4.52 (4H, m, αCH), 3.65 (2H, m), 3.31 (12H, m), 3.23 (6H, s, OCH₃), 2.96 (4H, d, J7.8 Hz), 2.78 (2H, m, ), 2.58 (2H, m), 1.86 (6H, m), 1.40 (10H, m).

¹³C NMR 75 MHz δ_{C}(CD₃OD) 178.50, 159.00, 138.05, 137.66, 130.54, 129.62, 129.50, 129.07, 128.79, 128.22, 73.90, 67.90, 58.64, 58.18, 56.02, 38.81, 30.06, 28.57, 28.36, 25.29. FTIR (KBr disc) νₘₐₓ (cm⁻¹) 3291.1, 3062.7, 3031.1, 2932.9, 2875.7, 2346.0, 1956.2, 1711.8, 1647.6, 1536.0, 1498.2, 1452.1, 1392.4, 1343.1, 1253.8, 1116.8, 1084.3, 1027.7, 916.0, 887.6, 748.6, 699.4, 595.5, 506.5.

### Example 16

### Preparation of Arginine Salt of TRI50C

Cbz-Phe-Pro-BoroMpg-OH obtained by the method of Example 5 (20.00 g, 38.1 mM) is dissolved in acetonitrile (200 ml) with stirring at room temperature. To this solution is added arginine as a 0.2M solution in distilled water (190 ml). The resultant clear solution is stirred for 2 hours at room temperature and then evacuated to dryness under vacuum with its temperature not exceeding 37° C. The resultant oil/tacky liquid is redissolved in 2L distilled water with warming to 37° C. for 2 hours. The solution is filtered through filter paper and evacuated to dryness, again with the temperature of the solution not exceeding 37° C. The resultant product is dried under vacuum overnight to normally yield a white brittle solid.

The salt was then dried under vacuum over silica to constant weight (72 h).

Yield: 10.54 g.

Microanalysis:

| C % Found | H % Found | N % Found | B % Found |
|---|---|---|---|
| (Calc.) | (Calc.) | (Calc.) | (Calc.) |
| 52.47 | 7.12 | 15.25 | 1.52 |
| (56.65) | (7.20) | (14.01) | (1.54) |

### Example 17

### UV/Visible Spectra of Arginine Salt of TRI50C

UV/Visible spectra of the salt resulting from the procedure of Example 15 were recorded in distilled water at 20° C. from 190 nm to 400 nm. TRI50C and the salt gave λₘₐₓ at 210 and 258 nm. The weight of the dried salt was then measured for the purposes of calculating the extinction coefficient. The λₘₐₓ at 258 nm was used. The extinction coefficient was calculated using the formula:—

A=εcl where A is the absorbance
C is the concentration
I the path length of the UV cell
and ε is the extinction coefficient.

Extinction coefficient: 406.

### Example 18

### Aqueous Solubility of Arginine Salt of TRI50C

The salt used in this Example was made using a modification of the process described in Example 16. The modified process differs from that described in that 100 mg of TRI 50c was used as starting material, the product of the redissolution in water was dried by freeze drying and the filtration was carried out through a 0.2 μm filter. The salt is believed to contain about 85% of R,S,R isomer.

To determine maximum aqueous solubility 25 mg of the dried salt were shaken in water at 37° C., the sample filtered and the UV spectrum measured. The salt left a white residue of undissolved material.

Solubility when dissolved at 25 mg/ml: 14 mM (10 mg/ml).

### Example 19

### Preparation of Lysine Salt of TRI50C

Cbz-Phe-Pro-BoroMpg-OH obtained by the method of Example 5 (20.00 g, 38.1 mM) is dissolved in acetonitrile (200 ml) with stirring at room temperature. To this solution is added L-lysine as a 0.2M solution in distilled water (190 mi). The resultant clear solution is stirred for 2 hours at room temperature and then evacuated to dryness under vacuum with its temperature not exceeding 37° C. The resultant oil/tacky liquid is redissolved in 3L distilled water with warming to 37° C. for 2 hours. The solution is filtered through filter paper and evacuated to dryness, again with the temperature of the solution not exceeding 37° C. The resultant product is dried under vacuum overnight to normally yield a white brittle solid. The product may be present as an oil or tacky solid (due to residual water), in which case it is then dissolved in ethyl acetate and evacuated to dryness to produce the product as a white solid.

The salt was then dried under vacuum over silica to constant weight (72 h).

Yield: 17.89.

Microanalysis:

| C % Found | H % Found | N % Found | B % Found |
|---|---|---|---|
| (Calc.) | (Calc.) | (Calc.) | (Calc.) |
| 57.03 | 7.43 | 10.50 | 1.72 |
| (59.11) | (7.36) | (10.44) | (1.61) |

### Example 20

### UV/Visible Spectra of Lysine Salt of TRI50C

UV/Visible spectra of the salt resulting from the procedure of Example 19 were recorded in distilled water at 20° C. from 190 nm to 400 nm. TRI50C and the salt gave λₘₐₓ at 210 and 258 nm. The weight of the dried salt was then measured for the purposes of calculating the extinction coefficient. The λₘₐₓ at 258 nm was used. The extinction coefficient was calculated using the formula:—

A=εcl where A is the absorbance
C is the concentration
I the path length of the UV cell
and ε is the extinction coefficient.

Extinction coefficient: 437.

### Example 21

### Aqueous Solubility of Lysine Salt of TRI50C

The salt used in this Example was made using a modification of the process described in Example 19. The modified process differs from that described in that 100 mg of TRI 50c was used as starting material, the product of the redissolution in water was dried by freeze drying and the filtration was carried out through a 0.2 μm filter. The salt is believed to contain about 85% of R,S,R isomer.

To determine maximum aqueous solubility 25 mg of the dried salt were shaken in water at 37° C., the sample filtered and the UV spectrum measured. The salt left a white residue of undissolved material.

Solubility when dissolved at 25 mg/ml: 13 mM (8.6 mg/ml).

### Example 22

### Preparation of N-Methyl-D-Glucamine Salt of TRI50C

Cbz-Phe-Pro-BoroMpg-OH obtained by the method of Example 5 (20.00 g, 38.1 mM) is dissolved in acetonitrile (200 ml) with stirring at room temperature. To this solution is added N-methyl-D-glucamine as a 0.2M solution in distilled water (190 ml). The resultant clear solution is stirred for 2 hours at room temperature and then evacuated to dryness under vacuum with its temperature not exceeding 37° C. The resultant oil/tacky liquid is redissolved in 500 ml distilled water with light warming for about 20 minutes. The solution is filtered through filer paper and evacuated to dryness, again with the temperature of the solution not exceeding 37° C., or freeze dried. The resultant product is dried under vacuum overnight to normally yield a white brittle solid.

The salt was then dried under vacuum over silica to constant weight (72 h).

Yield: 21.31 g.

Microanalysis:

| C % Found | H % Found | N % Found | B % Found |
|---|---|---|---|
| (Calc.) | (Calc.) | (Calc.) | (Calc.) |
| 56.67 | 7.28 | 7.74 | 1.63 |
| (56.67) | (7.41) | (7.77) | (1.50) |

### Example 23

### UV/Visible Spectra of N-Methyl-D-Glucamine Salt of TRI50C

UV/Visible spectra of the salt resulting from the procedure of Example 22 were recorded in distilled water at 20° C. from 190 nm to 400 nm. TRI50C and the salt gave λₘₐₓ at 210 and 258 nm. The weight of the dried salt was then measured for the purposes of calculating the extinction coefficient. The λₘₐₓ at 258 nm was used. The extinction coefficient was calculated using the formula:—

A=εcl where A is the absorbance
C is the concentration
I the path length of the UV cell
and ε is the extinction coefficient.

Extinction coefficient: 433.

### Example 24

### Aqueous Solubility of N-Methyl-D-Glucamine Salt of TRI50C

The salt used in this Example was made using a modification of the process described in Example 22. The modified process differs from that described in that 100 mg of TRI 50c was used as starting material, the product of the redissolution in water was dried by freeze drying and the filtration was carried out through a 0.2 μm filter. The salt is believed to contain about 85% of R,S,R isomer.

To determine maximum aqueous solubility 25 mg of the dried salt were shaken in water at 37° C., the sample filtered and the UV spectrum measured. The salt was observed to fully dissolve. The salt was comparatively soluble and so was redissolved at 50 mg/ml in the same manner previously described.

Solubility when dissolved at 25 mg/ml: 35 mM (25 mg/ml).

Solubility when dissolved at 50 mg/ml: 70 mM (50 mg/ml).

### Example 25

### Alternative Preparation of Arginine Salt of TRI50C

The arginine salt is formed simply by adding a slight molar excess of L-arginine to a solution of 0.2-0.3 mmol of TRI50c in 10 ml of ethyl acetate. The solvent is evaporated after one hour, and the residue is triturated twice with hexane to remove excess arginine.

### Example 26

### First Preparation of Calcium Salt of TRI 50C

Cbz-Phe-Pro-BoroMpg-OH (20.00 g, 38.1 mM) obtained by the method of Example 5 is dissolved in acetonitrile (200 ml) with stirring at room temperature. To this solution is added Ca(OH)₂ as a 0.1M solution in distilled water (190 ml). The resultant clear solution is stirred for 2 hours at room temperature and then evacuated to dryness under vacuum with its temperature not exceeding 37° C. The resultant product is a white brittle solid.

The salt was then dried under vacuum over silica to constant weight (72 h).

Yield: 17.69 g.

### Example 27

### Second Alternative Preparation of Calcium Salt of TRI 50C

50.0 g TRI 50c (95.2 mmol) were dissolved under stirring in 250 ml acetonitrile at room temperature and then cooled with an ice bath. To this ice cooled solution 100 ml of an aqueous suspension of 3.5 g (47.6 mmol) calcium hydroxide was added dropwise, stirred for 2.5 hours at room temperature, filtered and the resulting mixture evaporated to dryness, the temperature not exceeding 35° C. The clear yellowish oily residue was redissolved in 200 ml acetone and evaporated to dryness. The procedure of redissolving in acetone was repeated one more time to obtain colourless foam.

This foam was redissolved in 100 ml acetone, filtered and added dropwise to an ice cooled solution of 1100 ml petrol ether 40/60 and 1100 ml diethylether. The resulting colourless precipitate was filtered, washed two times with petrol ether 40/60 and dried under high vacuum, yielding 49.48 g of a colourless solid (92%), with a purity of 99.4% according to an HPLC measurement.

### Example 28

### UV/Visible Spectra of Calcium Salt of TRI 50C

UV/Visible spectra of the salt resulting from the procedure of Example 26 were recorded in distilled water at 20° C. from 190 nm to 400 nm. TRI 50C and the salt gave λₘₐₓ at 210 and 258 nm. The weight of the dried salt was then measured for the purposes of calculating the extinction coefficient. The λₘₐₓ at 258 nm was used. The extinction coefficient was calculated using the formula:—

A=εcl where A is the absorbance
C is the concentration
I the path length of the UV cell
and ε is the extinction coefficient.

Extinction coefficient: 955.

### Example 29

### Aqueous Solubility of Calcium Salt of TRI 50C

The salt used in this Example was made using a modification of the process described in Example 27. The modified process differs from that described in that 100 mg of TRI 50c was used as starting material, the product of the redissolution in water was dried by freeze drying and the filtration was carried out through a 0.2 μm filter. The salt is believed to contain about 85% of R,S,R isomer.

To determine maximum aqueous solubility 25 mg of the dried salt were shaken in water at 37° C., the sample filtered and the UV spectrum measured. The salt left a white residue of undissolved material.

Solubility when dissolved at 25 mg/ml: 5 mM (5 mg/ml).

### Example 30

### In Vitro Activity of Calcium Salt of TRI 50C

TRI 50c calcium salt was assayed as an inhibitor of human a-thrombin by an amidolytic assay (J. Deadman et al, J. Med. Chem. 38:15111-1522, 1995, which reports a Ki value of 7 nM for TRI 50b).

The inhibition of human a-thrombin therefore, was determined by the inhibition of the enzyme catalysed hydrolysis of three different concentrations of the chromogenic substrate S-2238.

200 μl of sample or buffer and 501l of S-2238 were incubated at 37° C. for 1 minute and 50 μl of human α-thrombin (0.25 NIHμ/ml) was added. The initial rate of inhibited and uninhibited reactions were recorded at 4.5 nm. The increase in optical density was plotted according to the method of Lineweaver and Burke. The Km and apparent Km were determined and Ki was calculated using the relationship.

$$V = \frac{V max}{1 + \frac{Km}{[ S ]} \cdot ( 1 + \frac{[ I ]}{Ki} )}$$

The buffer used contained 0.1M sodium phosphate, 0.2M NaCl, 0.5% PEG and 0.02% sodium azide, adjusted to pH 7.5 with orthophosphoric acid.

The samples consist of the compound dissolved in DMSO.

The reader is referred to Dixon, M and Webb, E. C., “Enzymes”, third edition, 1979, Academic Press, the disclosure of which is incorporated herein by reference, for a further description of the measurement of Ki.

TRI 50c calcium salt was observed to have a Ki of 10 nM.

### Example 31

### Preparation of Magnesium Salt of TRI 50C

TRI 50c (1.00 g, 1.90 mM) was dissolved in methanol (10 ml) and stirred at room temperature. To this solution was added magnesium methoxide (Mg(CH₃O)₂) in methanol (1.05 ml, 7.84 wt %). This solution was stirred for 2 hours at room temperature filtered and evacuated to 5 ml. Water (25 ml) was then added and the solution evacuated down to dryness to yield a white solid. This was dried over silica for 72 hours before being sent for microanalysis. Yield 760 mg.

¹H NMR 300 MHz, δ_{H}(CD₃C(O)CD₃) 7.14-7.22 (20H, m), 6.90 (2H, m), 4.89 (4H, m, PhCH₂O), 4.38 (2H, m), 3.40 (2H, br s), 2.73-3.17 (20H, broad unresolved multiplets), 1.05-2.10 (16H, broad unresolved multiplets).

¹³C NMR 75 MHz δ_{C}(CD₃C(O)CD₃) 206.56, 138.30, 130.76, 129.64, 129.31, 129.19, 129.09, 128.20, 128.04, 74.23, 73.55, 67.78, 58.76, 56.37, 56.03, 48.38, 47.87, 39.00, 25.42, 25.29. FFIR (KBr disc) νₘₐₓ (cm⁻¹) 3331.3, 3031.4, 2935.3, 2876.9, 2341.9, 1956.1, 1711.6, 1639.9, 1534.3, 1498.1, 1453.0, 1255.3, 1115.3, 1084.6, 1027.6, 917.3, 748.9, 699.6, 594.9, 504.5, 467.8.

### Example 32

### Solubility of TRI50C

The UV/visible spectra of TRI50c resulting from the procedure of Example 5 and its solubility were obtained as described above in relation to the salts. The solubility of TRI50c when dissolved at 50 mg/ml was 8 mM (4 mg/ml).

### Example 33

### Analysis of Sodium, Calcium, Magnesium and Zinc Salts of (R,S,R) TRI 50C

The following salts were prepared using a boronate:metal stoichiometry of n:1, where n is the valency of the metal, using (R,S,R) TRI 50c of higher chiral purity than that used to prepare the salts described in Examples 8, 11, 14, 18, 21, 24 and 29 .

A. Sodium Salt (Product of Example 9)

Analytical data
HPLC or LC/MS: HPLC betabasic C18 Column,
CH₃CN, Water
Estimated Purity: >95% by UV (λ₂₁₅ₙₘ)

Micro analysis:

| | | |
|---|---|---|
| | Calcd. | Found. |
| C: | 59.24 | 59.93 |
| H: | 6.44 | 6.47 |
| N: | 7.67 | 7.31 |
| Other: B: | 1.98 | 1.91 |
| Na: | 4.20 | 3.81 |

Physical Properties
Form: Amorphous solid
Colour: White
Melting Point: N/A
Solubility: Soluble in aqueous media
ca˜50 mg/ml
M_{w}: 547.40

B. Calcium Salt (Product of Example 26)

Analytical data
HPLC or LC/MS: HPLC betabasic C18 Column,
CH₃CN, Water
Estimated Purity: >95% by UV (λ₂₁₅ₙₘ)

Micro analysis:

| | | |
|---|---|---|
| | Calcd. | Found. |
| C: | 59.27 | 55.08 |
| H: | 6.48 | 6.43 |
| N: | 7.71 | 7.08 |
| Other: B: | 1.99 | 2.01 |
| Ca: | 3.68 | 3.65 |

Physical Properties
Form: Amorphous solid
Colour: White
Melting Point: N/A
Solubility: Soluble in aqueous media
ca˜4 mg/ml
M_{w}: 1088.89

C. Magnesium Salt (Product of Example 31)

Analytical data
HPLC or LC/MS: HPLC betabasic C18 Column,
CH₃CN, Water
Estimated Purity: >90% by UV (λ₂₁₅ₙₘ)

Micro analysis:

| | | |
|---|---|---|
| | Calcd. | Found. |
| C: | 60.44 | 57.25 |
| H: | 6.57 | 6.71 |
| N: | 7.83 | 7.45 |
| Other: B: | 2.01 | 2.02 |
| Mg: | 2.26 | 2.12 |

Physical Properties
Form: Amorphous solid
Colour: White
Melting Point: N/A
Solubility: Soluble in aqueous media
ca˜7 mg/ml
M_{w}: 1073.12

D. Zinc Salt (Product of Example 15)

Analytical data
HPLC or LC/MS: HPLC betabasic C18 Column,
CH₃CN, Water
Estimated Purity: >95% by UV (λ₂₁₅ₙₘ)

Micro analysis:

| | | |
|---|---|---|
| | Calcd. | Found. |
| C: | 58.21 | 56.20 |
| H: | 6.33 | 6.33 |
| N: | 7.54 | 7.18 |
| Other: B: | 1.94 | 1.84 |
| Zn: | 5.87 | 7.26 |

Physical Properties
Form: Amorphous solid
Colour: White
Melting Point: N/A
Solubility: Soluble in aqueous media
ca˜2 mg/ml
M_{w}: 1114.18
Notes:
The trigonal formula of the acid boronate is used in the calculated microanalyses. It is believed that a lower sodium salt solubility is reported in example 11 because the salt tested in example 11 had lower chiral purity.

Conclusion

The zinc, calcium and magnesium salts have all been prepared with a stoichiometry of one metal ion to two molecules of TRI 50c. The values found for the calcium and magnesium salts are close to and thus consistent with those calculated for this 1:2 stoichiometry. For the zinc salt an excess of zinc was found; nonetheless, the zinc salt comprises a significant proportion of acid boronate. The sodium salt has been prepared with a stoichiometry of one metal ion to one molecule of TRI 50c. The value found for the sodium salt is close to and thus consistent with that calculated for this 1:1 stoichiometry.

### Example 34

### Stability

An assay of TRI 50c and its sodium and lysine salts before and after drying.

1. Tabulated Results

TABLE 1

| Compound | Amount [μg/mL] | Purity (% area) |
|---|---|---|
| TRI 50c dry | 1000.0 | 82.00 |
| TRI 50c non-dried | 947.3 | 85.54 |
| TRI 50c Na salt dry | 1024 | 98.81 |
| TRI 50c Na salt non-dried | 1005.8 | 98.61 |
| TRI 50c Lys salt dry | 813.3 | 90.17 |
| TRI 50c Lys salt non-dried | 809.8 | 92.25 |

The purity of the acid was lowered by the drying process but the purity of the salts was less affected; the purity of the sodium salt was not significantly reduced. Large differences in response factors will reduce the actual impurity levels, however.

2. Analytical Procedure

2.1 Sample Preparation

TRI 50c and its Na, Li and Lys salts were weighed into HPLC vials and stored in a desiccator over phosphorus pentoxide for 1 week. For sample analysis, 5 mg of dried and non-dried material was weighed in a 5 mL volumetric flask and dissolved in 1 mL acetonitrile and filled up with demineralised water to 5 mL.

3. Data Evaluation

The quantitative evaluation was performed using an HPLC-PDA method.

4. Analytical Parameters

4.1 Equipment and Software

| | |
|---|---|
| Autosampler | Waters Alliance 2795 |
| Pump | Waters Alliance 2795 |
| Column oven | Waters Alliance 2795 |
| Detection | Waters 996 diode array, MS-ZQ 2000 single quad |
| Software version | Waters Millennium Release 4.0 |

4.2 Stationary Phase

| | |
|---|---|
| Analytical Column ID | S71 |
| Material | X-Terra ™ MS C₁₈, 5 μm |
| Supplier | Waters, Eschborn, Germany |
| Dimensions | 150 mm × 2.1 mm (length, internal diameter) |

4.3 Mobile Phase

| | |
|---|---|
| Aqueous phase: | A: H₂O + 0.1% |
| Organic phase: | C: ACN |

Gradient Conditions:

| Time | Flow | % A | % C |
|---|---|---|---|
| 0.00 | 0.5 | 90 | 10 |
| 27.0 | 0.5 | 10 | 90 |
| 27.1 | 0.5 | 90 | 10 |
| 30.0 | 0.5 | 90 | 10 |

This example indicates that the salts of the disclosure, particularly the metal salts, e.g. alkali metal salts, are more stable than the acids, notably TRI 50c.

### Example 35

### In-Vitro Assay as Thrombin Inhibitor of Magnesium Salt of TRI 50C

Thrombin Amidolytic Assay

TRI 50c magnesium salt (TRI 1405) was tested in a thrombin amidolytic assay.

Reagents:

Assay Buffer:

100 mM Na phosphate

200 mM NaCl (11.688 g/l)

0.5% PEG 6000 (5 g/l)

0.02% Na azide

pH 7.5

Chromogenic substrate S2238 dissolved to 4 mM (25 mg+10 ml) in water. Diluted to 50 uM with assay buffer for use in assay at 5 μM. (S2238 is H-D-Phe-Pip-Arg-pNA).

Thrombin obtained from HTI, via Cambridge Bioscience, and aliquoted at 1 mg/ml with assay buffer. Dilute to 100 ng/ml with assay buffer and then a further 1 in 3 for use in the assay.

Assay:

110 μl assay buffer

50 ul 5 μg/ml thrombin

20 μl vehicle or compound solution

5 min at 37° C.

20 μl 50 μM S2238

Read at 405 nm at 37° C. for 10 minutes and record Vmax

Results:

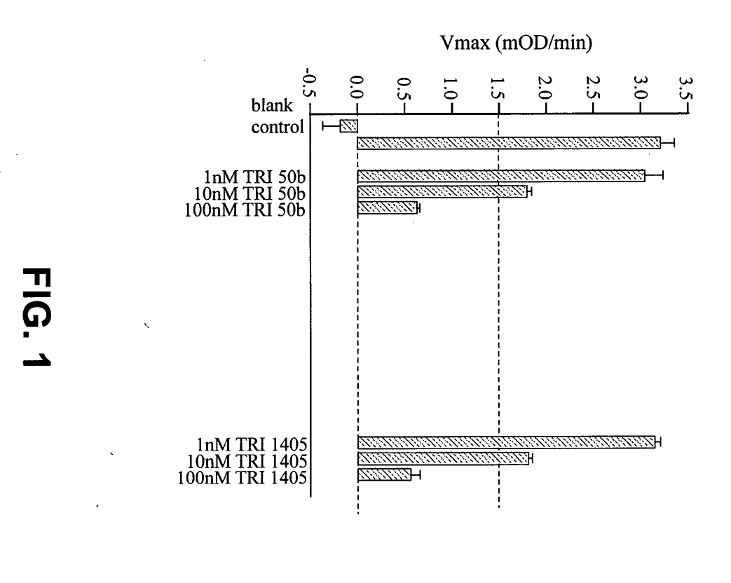

The results are presented in FIG. 1.

Discussion:

In this assay the magnesium salt of TRI 50c shows the same activity as TRI 50b as an external control.

### Example 36

### Intravenous Administration of TRI 50C Sodium Salt

The pharmacokinetics (PK) and pharmacodynamics (PD) of TRI 50c sodium salt were studied in beagle dogs following intravenous administration.

The PD was measured as thrombin time and APTT using an automated coagulometer. Plasma concentrations were measured using an LCMS/MS method.

TRI 50c monosodium salt (108.8g) was dissolved in 0.9% sodium chloride (100 ml) and dosed i.v. at 1.0 mg/kg (1.0 ml/kg over 30 seconds). Blood samples were taken into 3.8% tri-sodium citrate (1+8) at pre dose, 2, 5, 10, 20, 30, minutes post dose and then at 1, 2, 3, 4, 6, 8, 12 and 24 hours post dose. Plasma was prepared by centrifugation and frozen at minus 20° C. pending analysis.

Results

The sodium salt was tolerated well with no adverse events for the total duration of the study.

Male and female dogs responded similarly with a pharmacodynamic C max: at 2 minutes (thrombin time of 154 seconds raised from a base line of 14.3 seconds). Thrombin time was 26 seconds at one hour post dose.

There was an exceptionally good therapeutic ratio between the APTT and thrombin clotting time in dogs receiving the sodium salt at a dose of 1.0 mg/kg i.v. Thrombin clotting time was elevated 10.8 times above base line (154.4 seconds from 14.3 seconds) two minutes following dosing, compared to only 1.3 times elevation in the APTT (19 seconds to 25 seconds post dose).

### Example 37

### Residual n-Heptane of TRI 50C Calcium Salt

Salt prepared following the methods of Examples 1 and 3 was tested by headspace gas chromatography. Data are shown below:

Residual solvents: Headspace gas chromatography

| | |
|---|---|
| GC Parameter: | |
| Column: | DB-wax, 30 m, 0.32 mm ID, 5μ |
| Carrier Gas: | Helium 5.0, 80 kPas |
| Detector: | FID, 220° C. |
| Injector Temp: | 150° C. |
| Operating Conditions: | 35° C./7 min; 10° C./min up to 80° C./2 |
| 180° C./2 min | min; 40° C. up to |
| Injection volume: | 1 ml |
| Split: | On |
| Headspace Parameter: | |
| Oven temperature: | 70° C. |
| Needle temperature: | 90° C. |
| Transfer temperature: | 100° C. |
| Other parameters: | temper time: 15 min, GC-cycle time: 28 min, |
| | injection time: 0.03 min, duration: 0.4 min |

Calibration Standards: sample weight/dilution

| | | | concentration | area (average, |
|---|---|---|---|---|
| | weight (mg) | volume (ml) | (mg/ml) | n = 3) |
| standard | | | | |
| n-heptane | 103.12 | 100 | 1.0312 | 2757.74756 |
| sample no. | | | | |
| 1 | 100.84 | 5 | 20.17 | |
| 2 | 99.12 | 5 | 19.82 | |
| 3 | 100.03 | 5 | 20.01 | |

n-heptane

| sample | concentration (mg/ml) | content (%) |
|---|---|---|
| 1 | 0.0010 | 0.0048 |
| 2 | 0.0009 | 0.0044 |
| 3 | 0.0010 | 0.0050 |
| | 0.00095 | 0.005 |

### Example 38

### HPLC Chromatograms

TRI 50c monosodium salt made by the method of Examples 1, 2 & 3 and TRI 50c hemicalcium salt made by the method of Examples 1, 2 & 4 were analysed by HPLC chromatography.

1. Method

1.1 Equipment and Software

| | |
|---|---|
| Autosampler | Waters Alliance 2795 |
| Pump | Waters Alliance 2795 |
| Column oven | Waters Alliance 2795 |
| Detection | Waters 2996 diode array, MS-ZQ single quad |
| Software version | Waters Millennium 4.0 |

1.2 Stationary Phase

| | |
|---|---|
| Analytical Column ID | S-71 |
| Material | XTerra ™ MS C₁₈, 5 μm |
| Supplier | Waters, Eschborn, Germany |
| Dimension | 150 mm × 2.1 mm (length, ID) |
| Pre-column ID | no pre-column |

Xterra MS C₁₈, 5 μm is a column packing material supplied by Waters Corporation, 34 Maple Street, Milford, Mass. 01757, US and local offices, as in years 2002/2003. It comprises hybrid organic/inorganic particles, consisting of spherical particles of 5 μm size, 125 Å pore size and 15.5% carbon load.

1.3 Mobile Phase

| | |
|---|---|
| Aqueous phase: | A: H₂O + 0.1% HCOOH |
| Organic phase: | C: ACN |

H₂O = H₂O by Ultra Clear water purification system
ACN = gradient grade acetonitrile

Gradient Conditions

| time | | | flow | gradient |
|---|---|---|---|---|
| [min] | A % | C % | [mL/min] | shape |
| 0.0 | 90.0 | 10.0 | 0.5 | |
| 27.00 | 10.0 | 90.0 | 0.5 | linear |
| 27.10 | 90.0 | 10.0 | 0.5 | linear |
| 30.00 | 90.0 | 10.0 | 0.5 | linear |

1.4 Instrumental Parameters

| | |
|---|---|
| Flow | 0.5 mL-min⁻¹ |
| Temperature | 40 ± 5° C. |
| HPLC control | Waters Millennium Release 4.0 |
| Calculation | Waters Millenium 4.0 |

2. Parameters

2.1 Wavelength/Retention Time/Response Factors

TABLE
retention and detection parameter (k′ F: 0.5 ml/min, t0 = 0.9 ml/min)

| | | | | | Reciprocal |
|---|---|---|---|---|---|
| | RetTime | λ | | response factor | Response |
| Substance | [min] | [nm] | m/z | [area/μg] | factor |
| TRI 50c | 11.68 | 258 | 508.33 | 660 | 1 |
| Benzyl alcohol | 3.862 | 258 | n.d. | 1960 | 0.337 |
| Benzaldehyde | 6.13 | 258 | n.d. | 79939 | 0.0083 |
| Benzoic acid | 5.52 | 258 | n.d. | 5967 | 0.111 |
| Impurity I | 11.18 | 258 | 396.17 | 886 | 0.745 |
| Impurity II | 13.39 | 258 | 482.22 | 552 | 1.196 |

2.2 Linearity

Linearity Range 4000-10 μg/mL (detection UV 258 nm)

TABLE
Linearity data UV 258 nm

| calibration | area | target conc. | conc. found¹ |
|---|---|---|---|
| solution | [μAU's] | [μg/mL] | [μg/mL] |
| TRI 50c | 5353 | 10 | 20.44 |
| TRI 50c | 5301 | 10 | 20.37 |
| TRI 50c | 65809 | 100 | 113.35 |
| TRI 50c | 66365 | 100 | 114.17 |
| TRI 50c | 172019 | 250 | 270.43 |
| TRI 50c | 162587 | 250 | 256.48 |
| TRI 50c | 339503 | 500 | 518.13 |
| TRI 50c | 326912 | 500 | 499.51 |
| TRI 50c | 659257 | 1000 | 991.02 |
| TRI 50c | 647495 | 1000 | 973.63 |
| TRI 50c | 1322371 | 2000 | 1971.72 |
| TRI 50c | 1305196 | 2000 | 1946.32 |
| TRI 50c | 2724410 | 4000 | 4045.24 |

¹recalculated with linear equation

Linear Equation Parameters:

Y=6.75e+002 X−8.45e+003

r=0.99975

r²=0.99950

Linearity Range 10-0.10 μg/mL (detection SIR m/z 508,33)

TABLE
Linearity data SIR 508.33

| calibration | mean area | target conc. | conc. found¹ |
|---|---|---|---|
| solution | [μAU's] | [μg/mL] | [μg/mL] |
| TRI 50c | 2188860 | 0.01 | 0.022 |
| TRI 50c | 2702839 | 0.01 | 0.045 |
| TRI 50c | 3817226 | 0.1 | 0.094 |
| TRI 50c | 3833799 | 0.1 | 0.095 |
| TRI 50c | 23153550 | 1 | 0.947 |
| TRI 50c | 24646892 | 1 | 1.013 |
| TRI 50c | 223007852 | 10 | 9.765 |
| TRI 50c | 233753043 | 10 | 10.239 |

¹recalculated with linear equation

Equation Parameter

Y=2.27e+007 X+1.69e+006

r=0.99958

r²=0.99916

2.3 Quantitation Limit

The quantitation limit was determined using the signal to noise ratio criterion S/N>19,

UV 258 nm: 10 μg/mL

M/z 508.3: 0.1 μg/mL

2.4 Precision

| | Target | | | Retention |
|---|---|---|---|---|
| | concentration | | Amount | time |
| Injection | [μg/mL] | Area | [μg/mL] | [min] |
| 1 | 250 | 165805 | 261.24 | 11.690 |
| 2 | 250 | 168644 | 265.44 | 11.662 |
| 3 | 250 | 167858 | 264.27 | 11.686 |
| 4 | 250 | 166947 | 262.93 | 11.692 |
| 5 | 250 | 166925 | 262.89 | 11.679 |
| 6 | 250 | 166294 | 261.96 | 11.696 |
| Mean | | 167079 | 263.12 | 11.684 |
| Std. Dev. | | 1033 | 1.528 | 0.01 |
| % RSD | | 0.6 | 0.6 | 0.1 |

2.5 Robustness

TABLE
robustness data; Standard 250 μg/mL aqueous solution
(containing <1% ACN)

| calibration | | temp./time | | recovery |
|---|---|---|---|---|
| solution | | [° C./h] | area [μAU's] | [%] |
| 250 | μg/mL Tri50c | — | 172020 | — |
| 250 | μg/mL Tri50c | 4° C. 16 h | 166294 | 96.67 |
| 2.5 | μg/mL TRI50c | — | 88034891 | — |
| 2.5 | μg/mL TRI50c | 37° C. 4 h | 88833175 | 100.9 |

### REFERENCES

1. ICH HARMONISED TRIPARTITE GUIDELINE, TEXT ON VALIDATION OF ANALYTICAL PROCEDURES Recommended for Adoption at Step 4 of the ICH Process on 27 Oct. 1994 by the ICH Steering Committee

2. FDA Reviewer Guidance. Validation of chromatographic methods. Center for Drug Evaluation and Research. November 1994

3. USP 23. <621> Chromatography

4. L. Huber. Validation of analytical Methods. LC-GC International February 1998

5. Handbuch Validierung in der Analytik. Dr. Stavros Kromidas (Ed.) Wiley-VCH Verlag. 2000. ISBN 3-527-29811-8

3. Results

3.1 Sample Name: TRI 50c Monosodium Salt Injection Volume: 10 μL

| | Ret Time | | Area | Peak Height |
|---|---|---|---|---|
| Name | (Min) | Area % | [μAU's] | μAU |
| TRI 50c | 12.136 | 100.0000 | 604.27228 | 32.05369 |

3.2 Sample Name: TRI 50c Hemicalcium Salt Injection Volume: 10 μL

| | Ret Time | | Area | Peak Height |
|---|---|---|---|---|
| Name | (Min) | Area % | [μAU's] | μAU |
| TRI 50c | 12.126 | 100.0000 | 597.11279 | 32.29640 |

The disclosed methods have been used to obtain salts substantially free of C—B bond degradation products, in particular salts containing no such products in an amount detectable by HPLC, specifically the method of Example 38. The disclosed methods have been used to obtain salts substantially free of Impurity I, in particular containing no Impurity I in an amount detectable by HPLC, specifically by the method of Example 38. The disclosed methods have been used to obtain salts substantially free of Impurity IV, in particular containing no Impurity IV in an amount detectable by HPLC, specifically by the method of Example 38.

### Example 39

### Determination of Diastereomeric Excess

TRI 50b, crude, contains three chiral centres. Two of them are fixed by the use of enantiomerically pure amino acids ((R)-Phe and (S)-Pro). The third one is formed during the synthesis. The favoured epimer is the desired TRI 50b, Isomer I (R,S,R-TRI 50b). Both epimers of TRI 50b are clearly baseline separated by the HPLC method, thus allowing determination of the diasteromeric excess (de) of TRI 50b.

TRI 50d is not stable under the conditions applied for HPLC purity determination, but decomposes rapidly on sample preparation to TRI 50c, so that TRI 50d and TRI 50c show the same HPLC traces.

The two isomers of TRI 50c are not baseline separated in HPLC, but both isomers are clearly visible. This becomes obvious, when TRI 50b, crude (mixture of both isomers) is converted with phenylboronic acid to TRI 50c, crude. Both isomers of TRI 50c are observed in HPLC nearly at the same relation as before in TRI 50b, crude.

Upon synthesis of TRI 50d from TRI 50b, crude, only one diastereoisomer is precipitated. In this case HPLC shows only one peak for TRI 50c, where a very small fronting is observed. Precipitation from dichloromethane/diethylether removes the fronting efficiently. The level of removal of Isomer II cannot be quantified by this HPLC method. Therefore samples before reprecipitation and after one and two reprecipitations were esterified with pinacol and the resulting samples of TRI 50b analysed by HPLC. Thus a de of 95.4% was determined for the crude sample. The reprecipitated sample resulted in a de of 99.0% and finally the sample that was reprecipitated twice showed a de of 99.5%.

These results clearly show the preferred precipitation of Isomer I, whereas Isomer II remains in solution.

### Example 40

### Intravenous Administration Into Humans

Trial Protocol

TGN 255, the monosodium salt of TRI 50c, was administered intravenously to 18 healthy male subjects as a single intravenous dose (randomised double blind placebo study). The test consisted of three groups, each of six males. From each group 5 men were given the active ingredient and 1 was given a placebo.

Total I.V. administered doses were:
82 mg (7 mg intravenous bolus (over 30s) followed by an infusion of 25 mg/h for 3 hours).
130 mg (10 mg intravenous bolus (over 30s) followed by an infusion of 40 mg/h for 3 hours).
120 mg (by infusion of 40 mg/h for 3 hours).
   Trial Results

1) No clinically significant findings were detected in any safety assessments. There were no adverse clinical events of either a general or cardiovascular nature during the study period of 24 hours for any dose of TGN 255.

2) The disposition of TGN 255 followed a pattern with a short distribution phase occurring within 30 minutes after the end of infusion and was eliminated from the plasma with an overall terminal elimination half life of about 4 hours.

3) Intravenous infusion of TGN 255 induced a rapid, dose-related increase in the thrombin time (TT). Within 30 minutes after cessation of infusion there was a fall to a level at which clinically significant anticoagulation is not expected.

### Example 41

### Stability

This Example compares the stability of TRI 50c and TRI 50c calcium salt when filled into enteric-coated hard gelatin capsules (see Example 44).

1. Tabulated Results

| | | | | Purity |
|---|---|---|---|---|
| | | Climatic | Purity | (HPLC % |
| | | conditions | (HPLC % Area) | Area)³ |
| Compound. | Packing | 1.5 month⁰⁾ | T0 | T1 |
| TRI50c | capsules | 25° C./60% | 99 | 73.9 |
| | in blister | r.h.⁴ | | |
| TRI50c | capsules | 40° C./75% | 99 | 73.9 |
| | in blister | r.h | | |
| TRI50c | capsules¹ | 40° C./75% | 99 | 75.3 |
| | | r. h | | |
| TRI50c | capsules | 25° C./60% | 99.2²⁾ | 98.0 |
| Calcium Salt | in blister | r.h. | | |
| TRI50c | capsules | 40° C./75% | 99.2² | 97.2 |
| Calcium Salt | in blister | r.h | | |
| TRI50c | capsules¹ | 40° C./75% | 99.2² | 95.0 |
| Calcium Salt | | r.h | | |

Notes:
⁰⁾1.5 month storage at given conditions, samples were then stored at room temperature until analytical testing.
¹capsules stored at the respective climatic conditions without blister.
²⁾purity of the batch before storage.
³purity of the stored batch (capsules were poured out, the contents of the capsules were then analyzed).
⁴r.h. = relative humidity

2. Analytical Procedure
2.1 Sample Preparation
2.1.1 Assay of TRI 50c and Salts

TRI 50c-standard (free acid) was stored in a desiccator over phosphorus pentoxide for 2 days for drying. Afterwards, the reference standard was weighed in a volumetric flask and dissolved in a mixture of acetonitrile and water (25/75 v/v %). Aliquots of the resulting solution (ST 1A) were diluted successively with water as shown in the dilution scheme of table 4.

Stock- and Calibration solutions of Tri 50c

| | Net | | | | | | |
|---|---|---|---|---|---|---|---|
| | weight | Purity | | Dissolved | | Conc. | Calibr. |
| | mg | % | Salt-Factor | in ml | Solvent | [μg/ml] | [μg/ml] |
| ST 1 A | 40.8 | 98.23 | 1 | 10 | ACN/water | 4007.8 | C4000 |
| | | | | | 25/75 (v/v %) | | |
| | ml | ST | [μg/ml] | ad ml | Solvent | [μg/ml] | |
| ST 2 A | 5 | 1 A | 4007.8 | 10 | water | 2003.9 | C2000 |
| ST 3 A | 5 | 2 A | 2003.9 | 10 | water | 1001.9 | C1000 |
| ST 4 A | 5 | 3 A | 1001.9 | 10 | water | 501.0 | C500 |
| ST 5 A | 5 | 4 A | 500.9 | 10 | water | 250.5 | C250 |
| ST 6 A | 1 | 3 A | 1001.9 | 10 | water | 100.2 | C100 |
| ST 7 A | 1 | 6 A | 100.2 | 10 | water | 10.0 | C10 |

2.1.2 Impurity Profile of the Stored Capsules

The stored capsules of every batch at corresponding climatic condition were removed and 10 mg of the content was weighed in a 10 ml volumetric flask and dissolved in 10 ml of a mixture of acetonitrile/water (25/75 v/v %). These solutions were injected for impurity profile analysis and for quantification respectively.

3. Data Evaluation

The quantitative evaluation and the impurity profile analysis were performed using an HPLC-PDA method. The processing wavelength was set as 258 nm.

4. Analytical Parameters

4.1 Equipment and Software

| | |
|---|---|
| Autosampler | Waters Alliance 2795 |
| Pump | Waters Alliance 2795 |
| Column oven | Waters Alliance 2795 |
| Detection | Waters 996 diode array, extracted wavelength 258 nm |
| Software version | Waters Millennium Release 4.0 |

4.2 Stationary Phase

| | |
|---|---|
| Analytical Column ID | S71 |
| Material | X-Terra ™ MS C₁₈, 5 μm |
| Supplier | Waters, Eschborn, Germany |
| Dimensions | 150 mm × 2.1 mm (length, internal diameter) |

4.3 Mobile Phase

| | |
|---|---|
| Aqueous phase: | A: 0.1% HCOOH in water |
| Organic phase: | C: ACN |

Gradient Conditions:

| Time | Flow | % A | % C |
|---|---|---|---|
| 0.00 | 0.5 | 90 | 10 |
| 27.0 | 0.5 | 10 | 90 |
| 27.1 | 0.5 | 90 | 10 |
| 30.0 | 0.5 | 90 | 10 |

5. Impurity Profile Tables of TRI50C Ca Salt

Capsules in blister 25° C./60% r.h.

| | Amount | Retention Time | | | |
|---|---|---|---|---|---|
| Name | [μg/ml] | [min.] | Area | % Area | Height |
| Benzaldeh. | | 6,058 | 7927 | 1.29 | 392 |
| Tri50c | 930,903 | 11,686 | 601551 | 98.02 | 25135 |
| | | 19,199 | 839 | 0.14 | 89 |
| | | 19,498 | 1821 | 0.30 | 105 |
| | | 20,168 | 1581 | 0.26 | 158 |

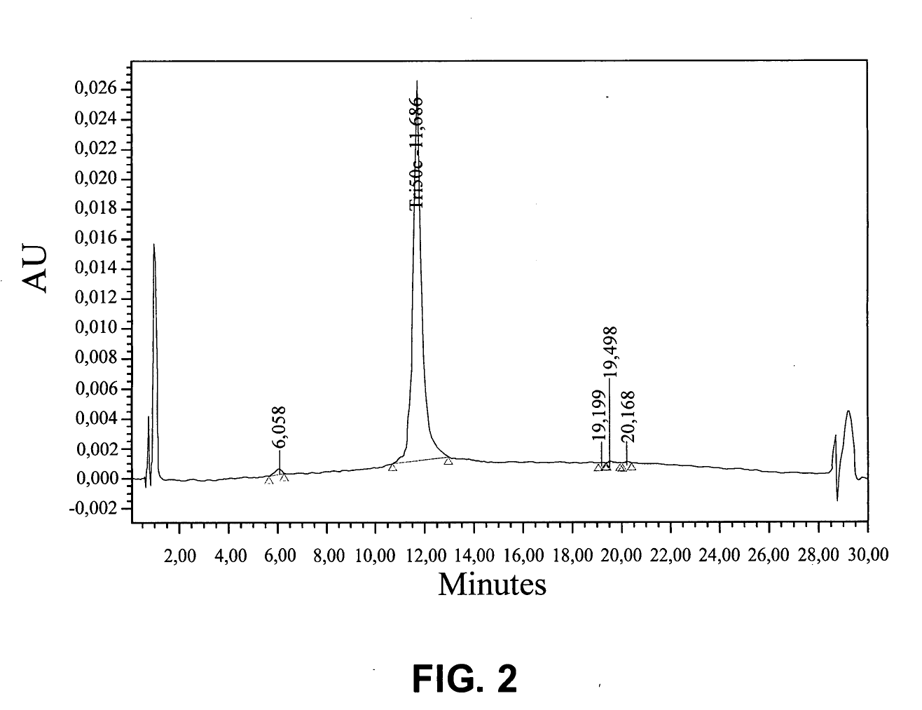

The corresponding HPLC trace is shown in FIG. 2

Capsules in blister 40° C./75% r.h.

| | Amount | Retention Time | | | |
|---|---|---|---|---|---|
| Name | [μg/ml] | [min.] | Area | % Area | Height |
| Benzaldeh. | | 6,060 | 12270 | 2.37 | 586 |
| Tri50c | 786,223 | 11,681 | 503867 | 97.19 | 21324 |
| | | 19,517 | 707 | 0.14 | 97 |
| | | 20,185 | 1614 | 0.31 | 169 |

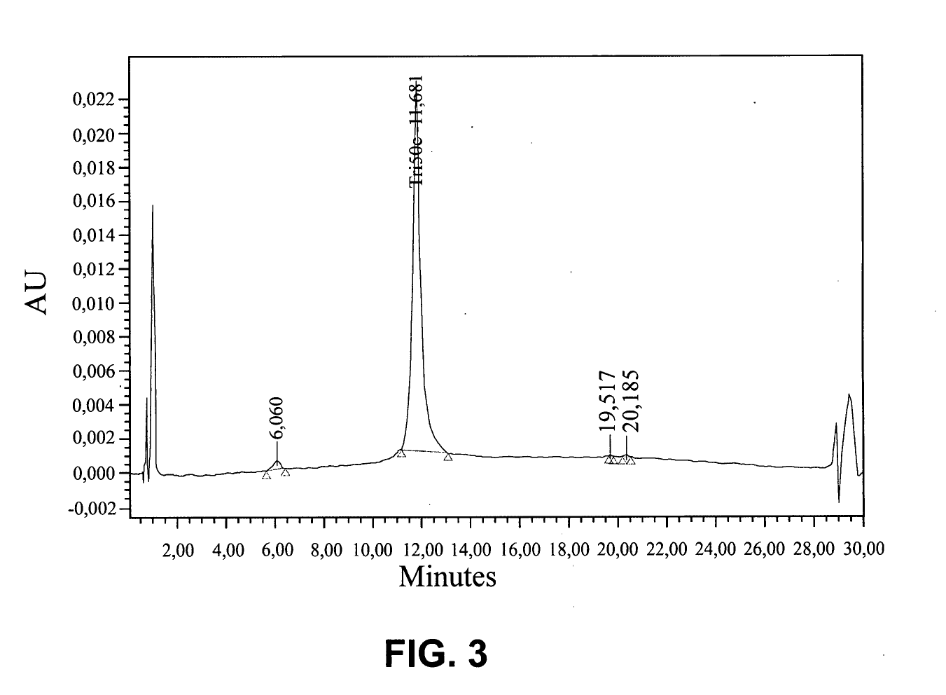

The corresponding HPLC time is shown in FIG. 3.

Capsules (no blister) 40° C./75% r.h.

| | Amount | Retention Time | | | |
|---|---|---|---|---|---|
| Name | [μg/ml] | [min.] | Area | % Area | Height |
| Benzaldeh. | | 6,041 | 19170 | 3.64 | 992 |
| Imp.I | | 10,897 | 4433 | 0.84 | 345 |
| Tri50c | 780,097 | 11,666 | 499730 | 94.96 | 21526 |
| | | 19,494 | 805 | 0.15 | 110 |
| | | 20,156 | 2100 | 0.40 | 176 |

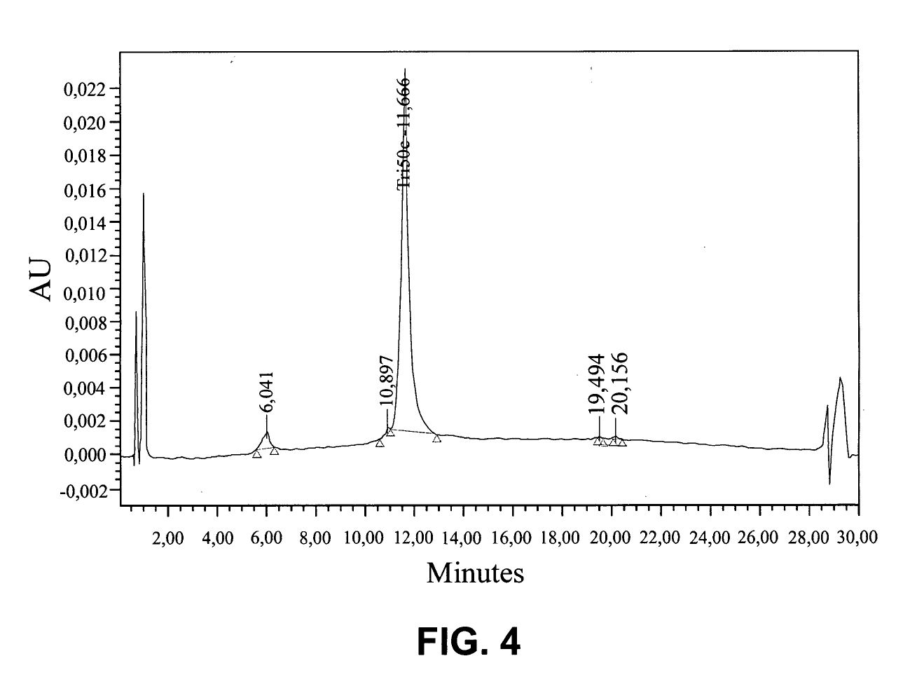

The corresponding HPLC trace is shown in FIG. 4.

### Example 42

### Intraduodenal Absorption in Rat

A. Preparation of Liquid Formulations of TRI 50c and Salt

1. Preparation of Buffer Solution pH 4.5

Place 1.48 g of sodium acetate (anhydrous) in a 1000 mL volumetric flask, add 16 mL 2N CH₃COOH, then add water and mix. Adjust the pH to 4,5 using 0.2 N NaOH and fill up with water.

2. Preparation of Buffer Solution pH 6.8 (USP)

Place 50.0 mL monobasic potassium phosphate 0.2 M in a 200 mL volumetric flask add 22.4 mL NaOH 0.2 M fill up with dest. Water. Check the pH and adjust if necessary.

3. Preparation of the Formulation

Place 10 mg of the relevant compound in an Eppendorf cup

Add 0.5 mL ethanol and shake for 10 minutes

Sonicate for 10 minutes

Add 1.5 mL of buffer

Shake for additional 15 minutes

Resulting target concentration: 5 mg/mL

B. Intraduodenal Studies

The intraduodenal studies were performed using male Wistar rats, approximately 8 weeks of age and weighing between 250 and 300 g.

Food was withheld overnight prior to dosing and returned approximately 2 hours post-dose. Water was available ad libitum.

Animals were anaesthetised using gaseous halothane. A small incision was made in the abdomen and the duodenum located. Each animal received a single administration of control or test article by injection directly into the duodenum, using a constant dose volume of 4 mL/kg. Following administration the incision was closed using surgical staples.

Individual dose volumes were based on individual body weights, obtained on the day of dosing.

Treatments employed for the study were as follows:

| | | Formulation | |
|---|---|---|---|
| | Dose level | concentration | Number |
| Treatment | (mg/kg) | (mg/mL) | of animals |
| TRI 50c control | 20 | 5 | 5 |
| Calcium salt | 20 | 5 | 5 |
| Potassium salt comparator | 20 | 5 | 5 |

Approximately 0.6 mL of blood was collected via a tail vein into 3.8% tri sodium citrate tubes approximately 48 hours prior to dosing and again at 0.5, 1, 2, 4 and 8 hours post-dose.

Plasma was prepared by centrifugation at 3000 rpm for 10 minutes at 4° C. Plasma was stored frozen (nominally −20° C.) prior to analysis in an automated coagulometer.

C. Results

TABLE 2
Mean thrombin time for intraduodenally dosed rats

| | Dose | Group mean thrombin time (s ± sd) at time (hour) | | | | | |
|---|---|---|---|---|---|---|---|
| Treatment | (mg/kg) | −48 | 0.5 | 1 | 2 | 4 | 8 |
| TRI 50c | 20 | 21.3 ± 2.69 | 42.1 ± 19.54 | 27.5 ± 9.42 | 23.5 ± 6.40 | 21.8 ± 2.33 | 21.5 ± 2.67 |
| control | | | | | | | |
| Calcium salt | 20 | 21.6 ± 1.77 | 42.0 ± 6.74 | 34.0 ± 1.89 | 22.6 ± 5.10 | 24.4 ± 2.41 | 22.4 ± 1.73 |
| Potassium salt | 20 | 20.0 ± 1.92 | 26.5 ± 3.64 | 24.4 ± 3.35 | 23.2 ± 0.83 | 23.2 ± 2.36 | 21.6 ± 0.70 |
| comparator | | | | | | | |

sd = standard deviation

### Example 43

### Oral Absorption in Rat

A. Preparation of Liquid Formulations of TRI 50c and Salt

The procedure of Example 42 was followed.

B. Oral Studies

The per-oral studies were performed using male Wistar rats, approximately 8 weeks of age and weighing between 250 and 300 g.

Food was withheld overnight prior to dosing and returned approximately 2 hours post-dose. Water was available ad libitum.

Each animal received a single administration of control or test article by oral gavage, using a constant dose volume of 4 mL/kg.

Individual dose volumes were based on individual body weights, obtained on the day of dosing.

Treatments employed for the study were as follows:

| | | Formulation | |
|---|---|---|---|
| | Dose level | concentration | Number of |
| Treatment | (mg/kg) | (mg/mL) | animals |
| TRI 50c control | 20 | 5 | 5 |
| Calcium salt | 20 | 5 | 5 |
| Potassium salt comparator | 20 | 5 | 5 |

Approximately 0.6 mL of blood was collected via a tail vein into 3.8% tri sodium citrate tubes approximately 48 hours prior to dosing and again at 0.5, 1, 2, 4 and 8 hours post-dose.

Plasma was prepared by centrifugation at 300 rpm for 10 minutes at 4° C. Plasma was stored frozen (nominally −20° C.) prior to analysis in an automated coagulometer.

C. Results

TABLE 3
mean thrombin times in the rat following oral administration

| | Dose | Group mean thrombin times (s ± sd) at time (hour) | | | | | |
|---|---|---|---|---|---|---|---|
| Treatment | (mg/kg) | −48 | 0.5 | 1 | 2 | 4 | 8 |
| TRI 50c | 20 | 22.9 ± 2.28 | 26.8 ± 1.96 | 23.3 ± 3.68 | 23.9 ± 2.25 | 23.1 ± 2.70 | 25.1 ± 0.33 |
| control | | | | | | | |
| Calcium salt | 20 | 23.4 ± 1.25 | 25.9 ± 3.05 | 25.7 ± 1.94 | 24.3 ± 0.98 | 25.0 ± 1.31 | 22.9 ± 3.46 |
| Potassium salt | 20 | 22.0 ± 1.40 | 24.7 ± 2.18 | 24.1 ± 1.87 | 22.9 ± 3.29 | 23.2 ± 1.24 | 23.8 ± 1.79 |
| comparator | | | | | | | |

sd = standard deviation

### Example 44

### Intraduodenal Variation

The thrombin times determined in example 42 were analysed to determine the standard deviation for increase in thrombin time, expressed as a percentage of the mean value (this is sometimes called the ‘coefficient of variation’). The variation for the Ca salt was calculated to be less than for TRI 50c, as shown in Table 4 below.

TABLE 4
Thrombin times in rats dosed intraduodenally

Time

| Product | 0 h | 0.5 h | increase | SD | SD % |
|---|---|---|---|---|---|
| TRI 50c | 23.70 | 40.02 | 16.32 | | |
| | 23.10 | 40.20 | 17.10 | | |
| | 16.85 | 23.60 | 6.75 | | |
| | 21.67 | 62.55 | 40.88 | | |
| | | Mean | 20.26 | 14.53 | 71.7% |
| Ca Salt | 21.97 | 35.32 | 13.35 | | |
| | 18.75 | 45.98 | 27.23 | | |
| | 23.57 | 37.27 | 13.70 | | |
| | 21.57 | 49.30 | 27.73 | | |
| | | Mean | 20.50 | 8.06 | 39.3% |

Conclusion

Examples 42 and 43 indicate that multivalent metal salts of boronic acids have a high oral bioavailability involving an unknown technical effect not linked to solubility.

Example 44 indicates that multivalent metal salts of boronic acids have a low variation in oral bioavailability involving an unknown technical effect not linked to solubility.

It is speculated that the technical effects may in some way involve coordination between the boronate group and the metal ion.

### Example 45

### Oral Administration in Dog

The pharmacokinetics (PK) and pharmacodynamics (PD) of TRI 50c (free acid) and its calcium salt were studied in beagle dogs following oral administration. Three female and three male dogs were used for each leg of the study. The weight range of the dogs was 8-18 kg.

The PD was measured as thrombin time and APTT using an automated coagulometer. Plasma concentrations were measured using an LCMS/MS method.

The calcium salt and TRI 50c were filled into gelatine capsules and enterically coated (HPMCP 55). The dose was tailored on an individual basis for each dog. Blood samples were taken into tri-sodium citrate as previously at pre dose, 0.5, 1, 1.5, 2, 3, 6, 8, 12, 16 and 24 hours post dose.

A. Results

A.1 Tolerance

The TRI 50c and the calcium salt were both tolerated well with no adverse events for the total duration of the study.

A.2 Calcium Salt

Unexpectedly high mean thrombin-clotting times were noted in dogs receiving the calcium salt. C max was observed three hours post dose with a mean thrombin clotting time of 80.5 seconds (raised from a base line of 15 seconds). There was still elevation of mean thrombin clotting times 8 hours post dose (mean of 20.2 seconds). All dogs responded dynamically following oral administration of the calcium salt, although there was some variability in response. All dogs dosed with the calcium salt achieved peak thrombin clotting times of up to 148 seconds, although the majority of animals (four out of six) achieved at least a four times elevation in peak thrombin time.

A.3 7RI 50c

Absorption as estimated by examination of dynamic response (TT) was variable. A peak thrombin time was noted 1.5 hours post dose (34.2 seconds from a base line of 15.4 seconds). Two animals failed to significantly absorb TRI 50c as estimated from their dynamic responses.

B. Activated Partial Thromboplastin Times

There were no significant changes in APTT from base line following administration of TRI 50c. There was a very slight mean elevation in APTT at 3 hours following administration of the calcium salt (14.5 seconds to 18 seconds at peak) this rise was deemed not to be clinically relevant.

C. Bioavailability

An estimation of bioavailability was achieved by a conversion of thrombin clotting times following administration of the calcium salt to estimated plasma concentrations.

Unexpectedly high absorption of the calcium salt was seen following oral absorption although there was some variability in responses; mean estimated bioavailability including two lower responders was 25 % and as high as 50% in some animals. TRI 50c was also well tolerated orally although the dynamic responses were significantly less than those for the calcium salt.

### Example 46

### Comparative Stability

The stability of TRI 50c sodium salt and TRI 50c sodium salt calcium salt have been studied in studies of similar design and conditions. In both studies the active pharmaceutical ingredient was stored in grip sealed double bags within a PE/PP screw cap closed cylinder. The packaging allows moisture transfer and the study was designed to allow the investigation into the effects of moisture and oxygen on the stability of these TRI 50c salts.

The results observed after 6 months storage are summarised in the tables below.

Results sodium-salt, data in % w/w

| | | | T = 1, | T = 1, | | T = 3, | T = 3, | | T = 6, | T = 6, |
|---|---|---|---|---|---|---|---|---|---|---|
| | | T = 1, | 25° C./60% | 40° C./75% | T = 3, | 25° C./ | 40° C./ | T = 6, | 25° C./60% | 40° C./ |
| | T = 0 | −20° C. | r.h. | r.h. | −20° C. | 60% r.h. | 75% r.h | −20° C. | r.h. | 75% r.h. |
| Appearance | Powdery | powdery | powdery | powdery | powdery | powdery | viscous | powdery | powdery | viscous |
| Color | White | white | white | white | white | white | brown | white | white | brown |
| Tri50c | 101.1 | 103.1 | 99.5 | 90.3 | 102.5 | 95.3 | 48.6 | 100.7 | 95.7 | 16.5 |
| (w/w %) | | | | | | | | | | |
| Tri50c | 101.5 | 104.3 | 103.6 | 94.5 | — | 100.4 | 52.2 | — | 100.5 | 17.4 |
| (w/w %, | | | | | | | | | | |
| LOD, corr.) | | | | | | | | | | |

Results
calcium-salt, data in % w/w

| | | | T = 1, | T = 1, | | T = 3, | T = 3, | | T = 6 | T = 6, |
|---|---|---|---|---|---|---|---|---|---|---|
| | | T = 1, | 25° C./ | 40° C./ | T = 3, | 25° C./60% | 40° C./ | T = 6 | 25° C./60% | 40° C./75% |
| | T = 0 | −20° C. | 60% r.h. | 75% r.h. | −20° C. | r.h. | 75% r.h. | −20° C. | r.h. | r.h. |
| Appearance | powdery, | powdery | powdery | powdery | powdery | powdery | powdery | powdery | powdery | powdery |
| | odourless | | | | | | | | | |
| Color | white | white | white | white | white | white | white | white | white | white |
| TRI 50c (% peak | 99.4(99.7) | 99.1 | 98.3 | 95.2 | 99.2(103.0)^{x} | 97.5(104.3) | 71.2(82.0) | 99.2(101.5)^{x} | 97.3(102.2) | 92.9(99.7) |
| area) | | (102.7)^{x} | (101.5) | (100.6) | | | | | | |
| Tri50c (w/w %, | | | | | | | | | | |
| LOD corrected) | | | | | | | | | | |

LOD = loss on drying

Discussion

The data in this example indicate that calcium salts of boronic acids are more stable than the corresponding sodium salts. It is contemplated that the same benefit may be provided by zinc.

It will be appreciated from the foregoing that the disclosure provides boronic acid salts useful for pharmaceutical purposes and which feature one or more of the following attributes: (1) improved hydrolytic stability; (2) improved stability against deboronation; and (3), in any event, not suggested by the prior art.

The selection of active ingredient for a pharmaceutical composition is a complex task, which requires consideration not only of biological properties (including bioavailability) but also of physicochemical properties desirable for processing, formulation and storage. Bioavailability itself is dependent on various factors, often including in vivo stability, solvation properties and absorption properties, each in turn potentially dependent on multiple physical, chemical and/or biological behaviours.

This specification further includes the subject matter of the following paragraphs:

1. A pharmaceutically acceptable base addition salt of an organoboronic acid of formula (XXX): 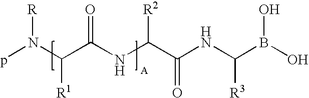
wherein:
P is hydrogen or an amino-group protecting moiety;
R is hydrogen or alkyl;
A is 0, 1 or 2;
R¹, R² and R³ are independently hydrogen, alkyl, cycloalkyl, aryl or —CH₂—R⁵;
R⁵, in each instance, is one of aryl, aralkyl, alkaryl, cycloalkyl, heterocyclyl, heteroaryl, or —W—R⁶, where W is a chalcogen and R⁶ is alkyl; and
where the ring portion of any of said aryl, aralkyl, alkaryl, cycloalkyl, heterocyclyl, or heteroaryl in R¹, R², R³ or R⁵ can be optionally substituted.

2. The salt of paragraph 1 which has an observed stoichiometry consistent with the organoboronic acid being in the form of a salt of which a single -OH group of the trigonally-represented boronyl group —B(OH)₂ is deprotonated or, in an alternative expression of the same deprotonation state, in which the boronyl group carries a single negative charge and is in a form selected from the group consisting of the following equilibrium species or a combination thereof:

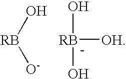

3. A pharmaceutically acceptable base addition salt of an organoboronic acid of formula (II) below, the salt having an observed stoichiometry consistent with the organoboronic acid being in the form of a salt comprising organoboronate anions and cations and of which a predominant portion has an anion:cation stoichiometry of about n:1, where n is the valency of the cation, formula (II) being: 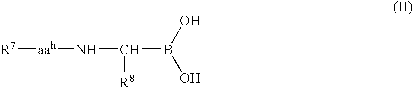
wherein

R⁷ is hydrogen or an amino-protecting group;

R⁸ is C₁-C₅ alkyl; and

aa^{h} is a hydrophobic amino acid.

4. A salt of any of paragraphs 1 to 3 wherein the organoboronic acid is N-(2-pyrazine) carbonyl-phenylalanine-leucine boronic acid.

5. A salt of any of paragraphs 1 to 4 which comprises a salt of the organoboronic acid with an alkali metal.

6. A salt of any of paragraphs 1 to 4 which comprises a salt of the organoboronic acid with a strongly basic organic nitrogen-containing compound.

7. A salt of any of paragraphs 1 to 4 which comprises a salt of the organoboronic acid with a multivalent metal.

8. A salt of any of paragraphs 1 to 4 which comprises a salt of the organoboronic acid with a metal.

9. A salt of any of paragraphs 1 to 4 which consists essentially of a monosodium salt.

10. A salt of any of paragraphs 1 to 4 which consists essentially of a hemicalcium salt.

11. A salt of any of paragraphs 1 to 4 which consists essentially of a salt of the organoboronic acid with an organic nitrogen-containing compound having a pKb of at least about 7.

12. A salt of any of paragraphs 1 to 11 when in solution.

13. A salt of paragraph 12 wherein the solution has as solvent a water-miscible organic solvent or water.

14. A method for preparing a product, the method comprising contacting an organoboronic acid drug, e.g. of formula (XXX), with a pharmaceutically acceptable base

15. The method of paragraph 14, wherein the pharmaceutically acceptable base provides cations having a valency n and the base is added in such an amount that the organoboronic acid and the cations are in a stoichiometry of n:1 (organoboronic acid:cations).

16. The method of paragraph 14 or paragraph 15 wherein the base is a basic metal compound.

17. The method of paragraph 16 wherein the metal is an alkali metal.

18. The method of paragraph 16 wherein the metal is an alkaline earth metal.

19. The method of paragraph 16 wherein the metal is a divalent metal.

20. The method of paragraph 14 or paragraph 15 wherein the base is an organic nitrogen-containing compound.

21. The method of paragraph 14 or paragraph 15 wherein the base is a basic sodium compound or a basic calcium compound.

22. The method of paragraph 14 or paragraph 15 wherein the base is an aminosugar or an amine of formula (XI): 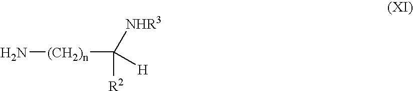
where n is from 1 to 6, R² is H, carboxylate or derivatised carboxylate, R³ is H, C₁-C₄ alkyl or a residue of a natural or unnatural amino acid

23. The method of paragraph 14 or paragraph 15 wherein the base is an aminosugar.

24. The method of paragraph 23 wherein the aminosugar is a ring-opened sugar.

25. The method of paragraph 23 wherein the aminosugar is a glucamine.

26. The method of paragraph 23 wherein the aminosugar is a cyclic aminosugar.

27. The method of any of paragraphs 14 to 26 wherein the organoboronic acid is optionally N-(2-pyrazine) carbonyl-phenylalanine-leucine boronic acid and the method further comprises formulating the product into an intravenous pharmaceutical formulation.

28. A method of stabilising an organoboronic acid drug, e.g. of formula (I) or formula (XXX), comprising providing it in the form of a pharmaceutically acceptable base addition salt thereof.

29. A method of paragraph 28, wherein the organoboronic acid is N-(2-pyrazine) carbonyl-phenylalanine-leucine boronic acid.

30. A method of formulating an organoboronic acid drug, e.g. of formula (I) or formula (XXX), to increase the stability of the drug species, comprising formulating the acid in the form of a pharmaceutically acceptable base addition salt thereof.

31. A pharmaceutically acceptable base addition salt of a boronic acid drug, e.g. N-(2-pyrazine) carbonyl-phenylalanine-leucine boronic acid, when in solution in a water miscible organic solvent.

32. A pharmaceutical formulation, whether in ready-to-use form or in a form requiring reconstitution prior to administration, adapted for intravenous administration and comprising the reaction product obtained by combining a pharmaceutically acceptable base with a boronic acid drug, e.g. N-(2-pyrazine) carbonyl-phenylalanine-leucine boronic acid.

33. A formulation of paragraph 32, wherein the boronic acid and cations of the salt are in an observed stoichiometry consistent with an acid:cation stoichiometry of about n:1, where n is the valency of the cations.

34. A lyophilisate of a pharmaceutically acceptable base addition salt of the compound N-(2-pyrazine) carbonyl-phenylalanine-leucine boronic acid.

35. A method of storing an organoboronic acid drug, e.g. of formula (I) or formula (XXX), for a period of at least six months, comprising providing the acid in the form of a reaction product thereof with a pharmaceutically acceptable base in a sealed container and storing it for at least six months at a temperature of at least 0° C.

36. A package comprising:
(i) a sealed container containing a boronic acid drug, e.g. of formula (I) or formula (XXX), in the form of a reaction product thereof with a pharmaceutically acceptable base; and
(ii) instructions permitting the container to be stored at a temperature of 10° C. or more for a period of 8 months or more.

37. A package of paragraph 36 wherein the instructions permit the container to be stored at a temperature of 15° C. or more for a period of 12 months or more.

38. A package of paragraph 36 or paragraph 37 wherein the boronic acid is N-(2-pyrazine) carbonyl-phenylalanine-leucine boronic acid.

39. A formulation of paragraph 32 or 33 wherein the salt is in the solid phase for reconstitution as an aqueous solution prior to administration by injection or infusion.

40. A formulation of paragraph 29 which is a solution ready for administration by injection or infusion.

41. A liquid intravenous formulation of a pharmaceutically acceptable base addition salt of the compound N-(2-pyrazine) carbonyl-phenylalanine-leucine boronic acid.

42. A formulation of paragraph 41 wherein the salt is a sodium salt.

43. A method of making an aqueous solution of a pharmaceutically acceptable base addition salt of a boronic acid, e.g. a drug for example of a compound of formula (I) or formula (XXX) below, the method comprising combining the salt and an aqueous solvent to form a solution having a pH of at least about 9.

44. A method of paragraph 43 which further comprises combining the solution with a pharmaceutically acceptable acid to decrease its pH.

45. A method of storing an organoboronic acid, e.g. a drug for example of formula (I) or formula (XXX), for a period of at least six months, comprising providing the acid in the form of a reaction product thereof with a pharmaceutically acceptable base in a sealed container and storing it for at least six months at a temperature of at least 0° C.

47. A method of paragraph 45 wherein the salt is stored for at least twelve months.

48. A method of paragraph 45 wherein the salt is stored in dry form.

49. A method of paragraph 45 wherein the salt is stored as a liquid formulation.

50. A method of paragraph 49 wherein the salt is stored as an aqueous solution.

51. A package of any of paragraphs 36 to 38 wherein the salt is in the solid phase.

52. A package of paragraph 51 wherein the salt is not a lyophilisate.

53. A package of any of paragraphs 36 to 38 wherein the salt is in the form of an aqueous preparation.

What is claimed is:

1. A pharmaceutically acceptable base addition salt of an organoboronic acid of formula (XXX): 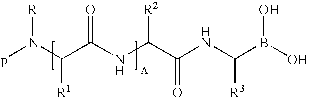
wherein:
P is hydrogen or an amino-group protecting moiety;
R is hydrogen or alkyl;
A is 0, 1 or 2;
R¹, R² and R³ are independently hydrogen, alkyl, cycloalkyl, aryl or —CH₂—R⁵;
R⁵, in each instance, is one of aryl, aralkyl, alkaryl, cycloalkyl, heterocyclyl, heteroaryl, or —W—R⁶, where W is a chalcogen and R⁶ is alkyl; and
where the ring portion of any of said aryl, aralkyl, alkaryl, cycloalkyl, heterocyclyl, or heteroaryl in R¹, R², R³ or R⁵ can be optionally substituted.

2. The salt of claim 1 which has an observed stoichiometry consistent with the organoboronic acid being in the form of a salt of which a single —OH group of the trigonally-represented boronyl group —B(OH)₂ is deprotonated or, in an alternative expression of the same deprotonation state, in which the boronyl group carries a single negative charge and is in a form selected from the group consisting of the following equilibrium species or a combination thereof: 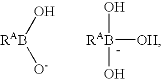
where R^{A} represents the following fragment of formula (XXX):

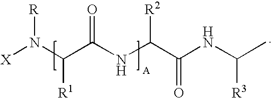

3. A pharmaceutically acceptable base addition salt of an organoboronic acid of formula (II) below, the salt having an observed stoichiometry consistent with the organoboronic acid being in the form of a salt comprising organoboronate anions and cations and of which a predominant portion has an anion:cation stoichiometry of about n:1, where n is the valency of the cation, formula (II) being: 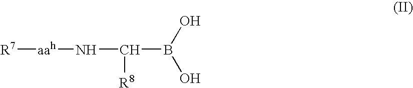
wherein
R⁷ is hydrogen or an amino-protecting group;
R⁸ is C₁-C₅ alkyl; and
aa^{h} is a hydrophobic amino acid.

4. A salt of claim 1 wherein the organoboronic acid is N-(2-pyrazine) carbonyl-phenylalanine-leucine boronic acid.

5. A salt of claim 2 wherein the organoboronic acid is N-(2-pyrazine) carbonyl-phenylalanine-leucine boronic acid.

6. A salt of claim 3 wherein the organoboronic acid is N-(2-pyrazine) carbonyl-phenylalanine-leucine boronic acid.

7. A salt of claim 2 which comprises a salt of the organoboronic acid with an alkali metal.

8. A salt of claim 2 which comprises a salt of the organoboronic acid with a strongly basic organic nitrogen-containing compound.

9. A salt of claim 2 which comprises a salt of the organoboronic acid with a multivalent metal.

10. A salt of claim 4 which consists essentially of a monosodium salt.

11. A salt of claim 4 which which consists essentially of a hemicalcium salt.

12. A salt of claim 5 which consists essentially of a salt of the organoboronic acid with a strongly basic organic nitrogen-containing compound.

13. A method for preparing a product, the method comprising contacting an organoboronic acid of formula (XXX) below with a pharmaceutically acceptable base, formula (XXX) being: 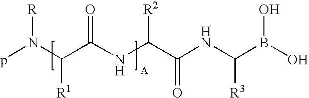
wherein:
P is hydrogen or an amino-group protecting moiety;
R is hydrogen or alkyl;
A is 0, 1 or 2;
R¹, R² and R³ are independently hydrogen, alkyl, cycloalkyl, aryl or —CH₂—R⁵;
R⁵, in each instance, is one of aryl, aralkyl, alkaryl, cycloalkyl, heterocyclyl, heteroaryl, or —W—R⁶, where W is a chalcogen and R⁶ is alkyl; and
where the ring portion of any of said aryl, aralkyl, alkaryl, cycloalkyl, heterocyclyl, or heteroaryl in R¹, R², R³ or R⁵ can be optionally substituted.

14. The method of claim 13, wherein the pharmaceutically acceptable base provides cations having a valency n and the base is added in such an amount that the organoboronic acid and the cations are in a stoichiometry of n:1 (organoboronic acid:cations).

15. The method of claim 13 wherein the base is a basic metal compound.

16. The method of claim 15 wherein the metal is sodium.

17. The method of claim 15 wherein the metal is a divalent.

18. The method of claim 13 wherein the base is an organic nitrogen-containing compound.

19. The method of claim 13 wherein the base is an aminosugar or an amine of formula (XI): 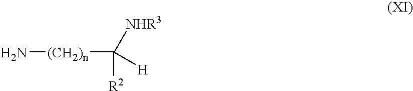
where n is from 1 to 6, R² is H, carboxylate or derivatised carboxylate, R³ is H, C₁-C₄ alkyl or a residue of a natural or unnatural amino acid.

20. The method of claim 14 wherein the base is an aminosugar.

21. The method of claim 20 wherein the aminosugar is a glucamine.

22. The method of claim 20 wherein the organoboronic acid is N-(2-pyrazine) carbonyl-phenylalanine-leucine boronic acid and the method further comprises formulating the product into an intravenous pharmaceutical formulation.

23. A method of stabilising an organoboronic acid of formula (XXX) below, comprising providing it in the form of a pharmaceutically acceptable base addition salt thereof, formula (XXX) being: 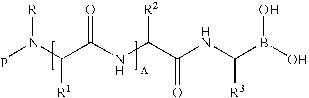
wherein:
P is hydrogen or an amino-group protecting moiety;
R is hydrogen or alkyl;
A is 0, 1 or 2;
R¹, R² and R³ are independently hydrogen, alkyl, cycloalkyl, aryl or —CH₂—R⁵;
R⁵, in each instance, is one of aryl, aralkyl, alkaryl, cycloalkyl, heterocyclyl, heteroaryl, or —W—R⁶, where W is a chalcogen and R⁶ is alkyl; and
where the ring portion of any of said aryl, aralkyl, alkaryl, cycloalkyl, heterocyclyl, or heteroaryl in R¹, R², R³ or R⁵ can be optionally substituted.

24. A method of claim 23, wherein the organoboronic acid is N-(2-pyrazine) carbonyl-phenylalanine-leucine boronic acid.

25. A pharmaceutical formulation, whether in ready-to-use form or in a form requiring reconstitution prior to administration, adapted for intravenous administration and comprising the reaction product obtained by combining a pharmaceutically acceptable base with N-(2-pyrazine) carbonyl-phenylalanine-leucine boronic acid.

26. A formulation of claim 25, wherein the boronic acid and cations of the salt are in an observed stoichiometry consistent with an acid:cation stoichiometry of about n:1, where n would be the valency of the cations.

27. The salt of claim 1, when provided in a sealed container and stored for a period of at least six months at a temperature of at least 0° C.
